(19) United States
(12) Patent Application Publication
(10) Pub. No.: US 2008/0299094 A1
(43) Pub. Date: Dec. 4, 2008

(54) Methods and Kits for Predicting Liver Fibrosis Progression Rate in Chronic Hepatitis C Patients

(75) Inventor: Ran Oren, Tel Aviv (IL)

Correspondence Address: Martin D. Moynihan;PRTSI, P.O.Box 16446, Arlington, VA, 22215, US

(73) Assignee: Medical Research Fund of Tel Aviv, Tel-Aviv (IL)

(21) Appl. No.: 11/631,340

(22) PCT Filed: Jun. 30, 2005
(86) PCT No.: PCT/IL2005/000700

§ 371 Date: Jul. 7, 2008

Related U.S. Application Data
(60) Provisional application No. 60/584,179, filed on Jul. 1, 2004.

(51) Int. Cl. A61K 35/12; C12Q 1/68; G01N 33/00; A61P 1/16

(52) U.S. Cl. 424/93.21; 435/6; 436/86

(57) ABSTRACT

Methods and kits for determining predisposition of an individual to develop fast progression rate of liver fibrosis are provided. Also provided are agents and pharmaceutical compositions useful in preventing fast progression of liver fibrosis and a method of identifying drug molecules which accelerate or induce liver fibrosis.

### FIELD AND BACKGROUND OF THE INVENTION

The present invention relates to methods and kits for predicting fibrosis progression rate in hepatitis C patients and, more particularly, to the use of such methods and kits in determining suitability of HCV patients for anti-viral treatment. Moreover, the present invention is of a method of preventing fast progression of liver fibrosis and/or cirrhosis.

Chronic hepatitis C is a common disease affecting approximately 170 million people worldwide (Lauer G M and Walker B D. Hepatitis C virus infection. N. Eng. J. Med. 2001; 345: 41-52). While most hepatitis C virus (HCV)-infected individuals exhibit a benign mode of infection, 15-20% of the infected individuals develop liver fibrosis which eventually progresses to end-stage liver cirrhosis (Seeff L B, et al., 2000. Ann. Intern. Med. 132: 105-11). The rate of fibrosis progression varies among HCV-infected individuals and is currently can not be predicted in a given individual.

A study of 2235 HCV-infected individuals revealed that while the median estimated duration from the infection date to the appearance of cirrhosis is 30 years, approximately 33% of the patients had progressed to cirrhosis in less than 20 years (Poynard T, et al., 1997; Natural history of liver fibrosis progression in patients with chronic hepatitis C. The OBSVIRC, METAVIR, CLINIVIR, and DOSVIRC groups. Lancet. 349: 825-32). These variations in fibrosis progression rate suggest that several host factors, i.e., factors related to the infected individual and not the hepatitis C virus itself, contribute to fibrosis progression.

For example, older age, male gender, alcohol intake and immunosuppressant therapy were found to be associated with a less favorable outcome in term of liver fibrosis [Poynard, 1997 (Supra)]. Other host factors, such as cigarette consumption, and body mass index which might affect the rate of fibrosis, are still under investigation (Feldman M, et al., 2002. Sleisenger & Fordtran's “Gastrointestinal and Liver disease” 7th Edition. SAUNDERS An Imprint of Elsevier Science).

Additionally, a considerable amount of evidence has been accumulated, implicating an important role for genetic factors in determining the natural history of liver diseases and the progression of liver fibrosis. These include genetic polymorphisms in genes encoding immunoregulatory proteins, proinflammatory cytokines, and fibrogenic factors (Bataller R, et al., 2003. Genetic polymorphisms and the progression of liver fibrosis: a critical appraisal. Hepatology. 37: 493-503). Thus, it was found that the apoE-epsilon 4 allele is associated with a protective outcome against liver damage caused by HCV (Wozniak M A, et al., 2002. Trent HCV Study Group. Apolipoprotein E-epsilon 4 protects against severe liver disease caused by hepatitis C virus. Hepatology. 36: 456-63). On the other hand, the C282Y polymorphism of the hemochromatosis gene (HFE) was found to be associated with cirrhosis (Smith B C, et al., 1998. Heterozygosity for hereditary hemochromatosis is associated with more fibrosis in chronic hepatitis C. Hepatology. 27: 1695-9). However, in another study which included 316 patients with hepatitis C, no significant difference was noted in the prevalence of HFE mutations between patients with compensated and end-stage liver disease (Tung B Y, et al., 2003. Hepatitis C, iron status, and disease severity: relationship with HFE mutations. Gastroenterology. 124: 318-26).

Other studies have found that a few polymorphic forms of the cytochrome P450 complex of enzyme (CYP450) are associated with various liver diseases. The most striking example is the association between the genetic polymorphism of CYP2E1 and the progression of alcoholic liver disease (Lee H C, et al., 2001. Association between polymorphisms of ethanol-metabolizing enzymes and susceptibility to alcoholic cirrhosis in a Korean male population. J. Korean Med. Sci. 16: 745-50).

CYP2D6, which belongs to the family of cytochrome P450 enzymes, involves in the metabolism of over 50 clinically important drugs (Hasler J A. 1999. Pharmacogenetics of cytochromes P450. Mol. Aspects. Med. 20: 12-24, 25-137). CYP2D6 includes several polymorphic forms, of which CYP2D6*3, CYP2D6*4 and CYP2D6*5 present poor drug metabolizers. The prevalence of such polymorphic alleles may account for poor drug metabolism in several individuals. For example, 5-10% of all Caucasian individuals are poor drug metabolizers. In this population, the prevalence of the CYP2D6*4 is as high as 23% [Hasler, 1999 (Supra)]. On the other hand, the prevalence of the other two common poor metabolizer alleles, CYP2D6*3 and CYP2D6*5, is much lower (2-5%).

Genetic studies of the CYP2D6 polymorphism revealed association of the active form of CYP2D6 with various carcinogenic processes such as cancer of the lung or larynx (Agundez J A, et al., 2001. Functionally active duplications of the CYP2D6 gene are more prevalent among larynx and lung cancer patients. Oncology. 61: 59-63). Similarly, the CYP2D6 poor metabolizer genotypes (i.e., CYP2D6*4 and CYP2D6*3) were found to be more frequent in healthy controls and HCV non-symptomatic carriers than in hepatitis/cirrhosis and hepatocellular carcinoma (HCC) patients [Silvestri L, et al., 2003. CYP enzyme polymorphisms and susceptibility to HCV-related chronic liver disease and liver cancer. Int. J. Cancer. 104: 310-7; Agundez J A, et al., 1995. CYP2D6 genes and risk of liver cancer. Lancet. 345(8953): 830-1].

However, all of the abovementioned studies have compared cirrhotic patients with non-cirrhotic patients, with no consideration to the rate of fibrosis progression which has a significant impact on the assessment of treatment in HCV-infected individuals.

The diagnosis of chronic hepatitis C infection is often suggested by abnormalities in alanine aminotransferase (ALT) levels and is established by enzyme immunoassay (EIA) followed by confirmatory determination of HCV RNA. Individuals who are infected with hepatitis C virus are monitored for disease progression using histopathological assessment of liver biopsies. According to the NIH CONCENSUS FROM JUNE 2002, patients with mild fibrosis, exhibiting portal F1, even in the presence of normal enzymes are candidates for antiviral therapy using PEG-interferon and Ribavirin (Shiffman M L. Et al., 2004; Peginterferon alfa-2a and ribavirin in patients with chronic hepatitis C who have failed prior treatment. Gastroenterology. 126: 1015-23). Among these patients, carriers of HCV genotypes type 1 and 4 are expected to response less efficiently such antiviral treatment (NIH Consens State Sci Statements. 2002; 19: 1-46)]. However, while determination of viral level and genotype as well as determination of liver enzymes involve non-invasive procedures, the determination of disease stage is based on recurrent liver biopsies, which can be associated with other complications resulting from general anesthesia, infections and the like.

There is thus a widely recognized need for, and it would be highly advantageous to have, a method of predicting the rate of fibrosis progression in HCV-infected individuals devoid of the above limitations.

### SUMMARY OF THE INVENTION

According to one aspect of the present invention there is provided a method of determining if an individual is predisposed to fast progression of liver fibrosis, the method comprising determining a presence or absence, in a homozygous or heterozygous form, of at least one fast progression liver fibrosis-associated genotype in the CYP2D6 locus or in neighboring loci of the individual, the neighboring loci being in linkage disequilibrium with the CYP2D6 locus, thereby determining if the individual is predisposed to fast progression of liver fibrosis.

According to another aspect of the present invention there is provided a kit for determining if an individual is predisposed to fast progression of liver fibrosis, the kit comprising at least one reagent for determining a presence or absence in a homozygous or heterozygous form, of at least one fast progression liver fibrosis-associated genotype in the CYP2D6 locus or in neighboring loci of the individual, the neighboring loci are in linkage disequilibrium with the CYP2D6 locus.

According to yet another aspect of the present invention there is provided a method of preventing fast progression of liver fibrosis in an individual in need thereof, the method comprising administering to the individual an agent capable of upregulating the expression level and/or activity of CYP2D6 in the liver of the individual, thereby preventing fast progression of liver fibrosis in the individual.

According to still another aspect of the present invention there is provided a method of determining if a drug molecule is capable of inducing or accelerating development of fast progression of liver fibrosis in an individual, comprising comparing a metabolism rate of the drug molecule by a CYP2D6 and a poor metabolizing variant of the CYP2D6, wherein poor metabolism of the drug molecule by the poor metabolizing variant of the CYP2D6 and not the CYP2D6 is indicative of its capability of inducing or accelerating development of fast progression of liver fibrosis in the individual

According to an additional aspect of the present invention there is provided a method of determining if an individual is predisposed to fast progression of liver fibrosis, the method comprising determining a presence or absence, in a homozygous or heterozygous form, of at least one fast progression liver fibrosis-associated genotype in a locus selected from the group consisting of CYP3A5, CYP2E1 and APO E or in neighboring loci of the individual, the neighboring loci being in linkage disequilibrium with the locus, thereby determining if the individual is predisposed to fast progression of liver fibrosis.

According to yet an additional aspect of the present invention there is provided a kit for determining if an individual is predisposed to fast progression of liver fibrosis, the kit comprising at least one reagent for determining a presence or absence in a homozygous or heterozygous form, of at least one fast progression liver fibrosis-associated genotype in a locus selected from the group consisting of CYP3A5, CYP2E1 and APO E or in neighboring loci of the individual, the neighboring loci are in linkage disequilibrium with the locus.

According to further features in preferred embodiments of the invention described below, the individual is infected with an hepatitis C virus.

According to still further features in the described preferred embodiments the at least one fast progression liver fibrosis-associated genotype in the CYP2D6 locus is an adenosine nucleotide-containing allele of the CYP2D6*4 SNP as set forth in SEQ ID NO:1.

According to still further features in the described preferred embodiments the at least one fast progression liver fibrosis-associated genotype encodes a truncated CYP2D6 polypeptide.

According to still further features in the described preferred embodiments the presence of the genotype is indicative of increased predisposition risk of developing fast progression of liver fibrosis in the individual.

According to still further features in the described preferred embodiments the presence of the genotype is indicative of increased predisposition risk of developing liver cirrhosis.

According to still further features in the described preferred embodiments the neighboring loci being in linkage disequilibrium with the CYP2D6 locus are included in the genomic sequence as set forth in SEQ ID NO:10.

According to still further features in the described preferred embodiments determining presence or absence of the genotype is effected using an SNP detection method selected from the group consisting of DNA sequencing, restriction fragment length polymorphism (RFLP analysis), allele specific oligonucleotide (ASO) analysis, Denaturing/Temperature Gradient Gel Electrophoresis (DGGE/TGGE), Single-Strand Conformation Polymorphism (SSCP) analysis, Dideoxy fingerprinting (ddF), pyrosequencing analysis, acycloprime analysis, Reverse dot blot, GeneChip microarrays, Dynamic allele-specific hybridization (DASH), Peptide nucleic acid (PNA) and locked nucleic acids (LNA) probes, TaqMan, Molecular Beacons, Intercalating dye, FRET primers, AlphaScreen, SNPstream, genetic bit analysis (GBA), Multiplex minisequencing, SNaPshot, MassEXTEND, MassArray, GOOD assay, Microarray miniseq, arrayed primer extension (APEX), Microarray primer extension, Tag arrays, Coded microspheres, Template-directed incorporation (TDI), fluorescence polarization, Colorimetric oligonucleotide ligation assay (OLA), Sequence-coded OLA, Microarray ligation, Ligase chain reaction, Padlock probes, Rolling circle amplification, and Invader assay.

According to still further features in the described preferred embodiments the kit further comprising packaging material packaging at least one reagent and a notification in or on the packaging material, the notification identifying the kit for use in determining if an individual is predisposed to fast progression of liver fibrosis.

According to still further features in the described preferred embodiments the at least one reagent is an antibody capable of differentially binding at least one polymorph of a CYP2D6 protein set forth by SEQ ID NO:4.

According to still further features in the described preferred embodiments the individual is suffering from a disease selected from the group of an hepatitis viral infection, an hepatotoxicity, a liver cancer, a non alcoholic fatty liver disease (NAFLD), an autoimmune disease, a metabolic liver disease, and a disease with secondary involvement of the liver.

According to still further features in the described preferred embodiments the hepatitis viral infection is caused by a virus selected from the group consisting of hepatitis C virus, hepatitis B virus, and hepatitis D virus.

According to still further features in the described preferred embodiments the hepatotoxicity is alcohol-induced hepatotoxicity and/or drug-induced hepatotoxicity.

According to still further features in the described preferred embodiments the autoimmune disease is selected from the group consisting of autoimmune hepatitis (AIH), primary biliari cirrhosis (PBC) and primary sclerosing cholangitis (PSC).

According to still further features in the described preferred embodiments the metabolic liver disease is selected from the group consisting of Hemochromatosis, Wilson's disease and alpha 1 anti trypsin.

According to still further features in the described preferred embodiments the disease with secondary involvement of the liver is celiac disease and/or amyloidosis.

According to still further features in the described preferred embodiments upregulating is effected by at least one approach selected from the group consisting of:

(a) expressing in liver cells of the individual an exogenous polynucleotide encoding at least a functional portion of CYP2D6;

(b) increasing expression of endogenous CYP2D6 in liver cells of the individual;

(c) increasing endogenous CYP2D6 activity in liver cells of the individual; and

(d) administering CYP2D6-expressing cells into the liver of the individual.

According to still further features in the described preferred embodiments the CYP2D6 is a polypeptide at least 75% identical to the polypeptide set forth by SEQ ID NO:4 as determined using the BlastP software of the National Center of Biotechnology Information (NCBI) using default parameters.

According to still further features in the described preferred embodiments the CYP2D6 is set forth by SEQ ID NO:4.

According to still further features in the described preferred embodiments the polynucleotide is set forth by SEQ ID NO:5.

According to still further features in the described preferred embodiments the poor metabolizing variant of the CYP2D6 is selected from the group consisting of CYP2D6*4, CYP2D6*3, and CYP2D6*5.

According to still further features in the described preferred embodiments the CYP2D6 is expressed from a polynucleotide encoding at least a functional form of CYP2D6.

According to still further features in the described preferred embodiments the poor metabolizing variant of said CYP2D6 is expressed from a polynucleotide encoding a truncated CYP2D6 polypeptide.

According to still further features in the described preferred embodiments the at least one fast progression liver fibrosis-associated genotype in said CYP3A5 locus is an adenosine nucleotide-containing allele at nucleotide coordinate 174 of SEQ ID NO:18.

According to still further features in the described preferred embodiments the at least one fast progression liver fibrosis-associated genotype in said CYP2E1 locus is a Thymidine nucleotide-containing allele at nucleotide coordinate 1772 of SEQ ID NO:17.

According to still further features in the described preferred embodiments the said at least one fast progression liver fibrosis-associated genotype in said APO E locus is a Cytosine nucleotide-containing allele at nucleotide coordinate 55 of SEQ ID NO:19.

The present invention successfully addresses the shortcomings of the presently known configurations by providing a method of determining predisposition to fast progression of liver fibrosis.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a graph adopted from Poynard T., et al., 2001; J. of Hepatology, 34: 730-739, illustrating the progression rate to cirrhosis as a function of the duration of infection and the age at infection.

### DESCRIPTION OF THE PREFERRED EMBODIMENTS

The present invention is of a method of determining predisposition to fast progression of liver fibrosis which can be used to determine suitability of hepatitis C infected individuals to antiviral therapy. In addition, the present invention provides a method and pharmaceutical compositions useful in preventing fast progression of liver fibrosis.

The principles and operation of the methods of determining predisposition and prevention of fast progression of liver fibrosis according to the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Chronic hepatitis C is a common disease affecting approximately 170 million people worldwide [Lauer and Walker, 2001 (Supra)]. Among HCV-infected individuals 15-20% develop liver fibrosis which often progresses to end-stage liver cirrhosis [Seeff, 2000 (Supra)]. Currently, there are no means of predicting which of the HCV-infected individuals will develop liver fibrosis. In addition, the duration from the time of infection to the appearance of cirrhosis varies between individuals and can not be predicted [(Poynard, 1997 (Supra)]. Moreover, several host factors such as older age, male gender, alcohol intake and immunosuppression therapy were found to be associated with liver fibrosis and cirrhosis [Poynard, 1997 (Supra)]. Other host factors, such as cigarette consumption, and body mass index which might affect the rate of fibrosis, are still under investigation [Feldman, 2002 (Supra)].

Prior attempts to identify genetic factors contributing to liver fibrosis revealed association of genetic polymorphism in the hemochromatosis gene (HFE) [Smith, 1998 (Supra)], the glutathione S-transferase gene (Ghobadloo S M, et al., 2004. J. Gastrointest. Surg. 8: 423-7), the IL1RA cytokine gene (Bahr M J et al., 2003. Liver Int. 23: 420-5) and the myeloperoxidase (MPO) gene (Reynolds W F, et al., 2002. Genes Immun. 3: 345-9) with liver cirrhosis. Other studies revealed contradicting results regarding the role of the C282Y polymorphism in liver cirrhosis [Smith, 1998 (Supra); Tung, 2003 (Supra)]. However, all of these studies compared the prevalence of genetic polymorphisms between cirrhotic patients and non-cirrhotic patients, with no consideration to the rate of fibrosis progression which has a significant impact on the assessment of treatment in HCV-infected individuals.

While reducing the present invention to practice, the present inventor has compared the prevalence of genetic polymorphisms among HCV-infected individuals which progress fast (i.e., “fast fibrosers”) or slow (i.e., “slow fibrosers”) towards liver fibrosis and cirrhosis and associated genotypes in the CYP2D6 locus with fast progression of liver cirrhosis.

As is shown in Example 1 of the Examples section which follows, the present study conclusively shows that the CYP2D6*4 allele encoding a poor metabolizer form of CYP2D6 is more prevalent among fast liver fibrosers than among slow liver fibrosers. Moreover, the frequency of individuals heterozygous and/or homozygous of the CYP2D6*4 allele is higher among fast liver fibrosers than among slow liver fibrosers, suggesting the use of the CYP2D6*4 allele in determining predisposition to fast liver fibrosis.

Thus, according to one aspect of the present invention there is provided a method of determining if an individual is predisposed to fast progression of liver fibrosis.

As used herein, the term “individual” includes both young and old human beings of both sexes. Preferably, this term encompasses individuals who are at risk to develop liver fibrosis, for example, individuals who are infected with hepatitis C virus, or with other hepatotoxic viruses (e.g., hepatitis B, D), individuals who suffer from hepatotoxicity due to consumption of more than 2 units of alcohol daily or hepatotoxic drugs, individuals having liver cancer, non alcoholic fatty liver disease (NAFLD), an autoimmune disease such as autoimmune hepatitis (AIH), primary biliari cirrhosis (PBC) and primary sclerosing cholangitis (PSC), a metabolic liver disease such as Hemochromatosis, Wilson's disease and alpha 1 anti trypsin and/or a disease with secondary involvement of the liver such as celiac disease or amyloidosis. Preferably, the individual of the present invention is a human being which is infected with the hepatitis C virus.

As used herein, the term “predisposed” when used with respect to fast progression of liver fibrosis refers to an individual which is more likely to develop fast progression of liver fibrosis than a non-predisposed individual.

Liver fibrosis is characterized by presence of fibrotic tissue (i.e., a scar tissue of dead cells) within the liver tissue. Liver fibrosis is often a result of chronic inflammation of the liver due to, for example, infection with hepatitis C virus. Chronic inflammation leads to changes in liver structure, to slowing of blood circulation, and necrosis (i.e., death) of liver cells. Methods of evaluating the presence of liver fibrosis are known in the arts. For example, as described in Example 1 of the Examples section which follows, the presence of liver fibrosis can be detected using histopathology findings of liver biopsy. Thus, the grade and stage of liver biopsy can be assessed according to the Batts and Ludwig system (B&L) consisting of the following classifications: 1—Portal fibrosis; 2—periportal fibrosis; 3—septal fibrosis; 4—cirrhosis. In addition, liver fibrosis can be detected using clinical findings such as signs of portal hypertension as well as laboratory and appropriate radiology findings.

The phrase “fast progression of liver fibrosis” as used herein refers to the development of liver fibrosis within a time period which is shorter than expected according to the individual's age at the time of infection based on the Poynard's fibrosis progression model (Poynard et al., 2001. Rates and risk factors of liver fibrosis progression in patients with chronic hepatitis C. J. Hepatol. 34: 730-9). For example, a normal rate of progression of liver fibrosis in an individual younger than 20 years of age is 40 years. On the other hand, individuals who are infected at the age of 40 or older will develop liver fibrosis following 10-20 years from the time of infection. Thus, fast progressing liver fibrosis is defined herein as fibrosis which occurs over a time period which is at least 5 years shorter than expected, more preferably, at least 10 years, most preferably, at least 20 years shorter than expected according to the Poynard's fibrosis progression model.

The method is effected by determining a presence or absence, in a homozygous or heterozygous form, of at least one fast progression liver fibrosis-associated genotype in the CYP2D6 locus or in neighboring loci of the individual which are in linkage disequilibrium with the CYP2D6 locus, thereby determining if the individual is predisposed to fast progression of liver fibrosis.

As used herein the term “CYP2D6 locus” refers to a specific DNA sequence region in the human genome encompassing a gene coding for the cytochrome P450/family 2/subfamily 2/polypeptide 6 (CYP2D6) and located on the long arm of chromosome 22 (22q13.1) between two cytochrome P450 pseudogenes. The CYP2D6 protein is a monooxygenase enzyme involved in the metabolism of over 50 clinically important drugs [Hasler, 1999 (Supra)], including debrisoquine, an adrenergic-blocking drug, sparteine and propafenone, both anti-arrythmic drugs, and amitryptiline, an anti-depressant drug. Genetic polymorphisms in the CYP2D6 gene results in various forms of the CYP2D6 protein of which CYP2D6*3, CYP2D6*4, and CYP2D6*5 (Hersberger M, et al., 2000. Clin. Chem. 46: 1072-7) represent poor drug metabolizers (Nelson D R Cytochrome P450 nomenclature. Methods Mol. Biol. 1998; 107:15-24). The terms “homozygous” or “heterozygous” refer to two identical or two different alleles, respectively, of a certain polymorphism.

The term “polymorphism” refers to the occurrence of two or more genetically determined variant forms (alleles) of a particular nucleic acid or a nucleic acid sequence (e.g., gene) at a frequency where the rarer (or rarest) form could not be maintained by recurrent mutation alone. A non-limiting example of a polymorphism is the G/A substitution at position 3465 of the CYP2D6 gene (SEQ ID NO:6, GenBank Accession No. M33388) which is set forth by SEQ ID NO:1 and encodes the CYP2D6*4 polymorphism.

As is shown in Table 3 of the Examples section which follows, the present inventor has uncovered that the adenosine nucleotide-containing allele of the CYP2D6*4 SNP as set forth in SEQ ID NO:1 encoding the poor metabolizer polymorph is more prevalent among fast fibrosers, i.e., individuals exhibiting fast progression of liver fibrosis than among slow fibrosers, i.e., individuals in which the progression of liver fibrosis (p value=0.0166). Moreover, as is shown in Tables 3 and 4 of the Examples section which follows, individuals heterozygous and/or homozygous to poor metabolizer allele (i.e., the adenosine nucleotide-containing allele of the CYP2D6*4 SNP) were significantly more prevalent in the fast fibroser group than in the slow fibroser group (p value=0.022, OR=11.7, 95% C.I. 1.4-95.27).

In addition, as is further shown in Tables 7 and 8 and described in Example 2 of the Examples section which follows, when additional 32 chronic hCV patients were tested for the presence or absence of the CYP2D6*4 SNP, the overall difference in the frequency of the CYP2D6*4 allele was about 33% in the fast fibroser group and only about 13% in the slow fibroser group. In addition, the overall frequency of the CYP2D6*4 carriers was about 51% among the fast fibroser group and only about 22% among the slow fibroser group.

The CYP2D6*4 polymorphism encodes a splice mutation in which the guanine nucleotide of the AG splice acceptor site at the junction between the third intron and the forth exon of the CYP2D6 gene is substituted with an adenosine nucleotide (3465G→A in GenBank Accession No. M33388), resulting in a truncated CYP2D6 protein.

Thus, according to preferred embodiments of the present invention the at least one fast progression liver fibrosis-associated genotype in the CYP2D6 locus encodes a truncated form of the CYP2D6 polypeptide (having a deletion of at least one internal or terminal amino acid region), such as the CYP2D6*4, CYP2D6*3, and/or CYP2D6*5 polymorphs. Preferably, the fast progression liver fibrosis-associated genotype of the present invention is the adenosine nucleotide-containing allele of the CYP2D6*4 SNP as set forth in SEQ ID NO:1.

As is mentioned hereinabove, the method of the present invention can also be effected by identifying SNPs which are in neighboring loci and are in linkage disequilibrium with the fast progression liver fibrosis associated SNPs in the CYP2D6 locus.

The phrase “neighboring loci” is used herein to describe DNA sequences (either genes or intergenic sequences) that are in close vicinity of the CYP2D6 locus and that include other SNPs that are in linkage disequilibrium with SNPs in the CYP2D6 locus.

The phrase “linkage disequilibrium” (LD) is used to describe the statistical correlation between two neighboring polymorphic genotypes. Typically, LD refers to the correlation between the alleles of a random gamete at the two loci, assuming Hardy-Weinberg equilibrium (statistical independence) between gametes. LD is quantified with either Lewontin's parameter of association (D′) or with Pearson correlation coefficient (r) [Devlin B, Risch N. (1995). A comparison of linkage disequilibrium measures for fine-scale mapping. Genomics. 29: 311-322.]. Two loci with a LD value of 1 are said to be in complete LD. At the other extreme, two loci with a LD value of 0 are termed to be in linkage equilibrium. Linkage disequilibrium is calculated following the application of the expectation maximization algorithm (EM) for the estimation of haplotype frequencies [Slatkin M, Excoffier L. (1996). Testing for linkage disequilibrium in genotypic data using the Expectation-Maximization algorithm. Heredity. 76: 377-83.]. Preferably, LD values according to the present invention for neighboring genotypes/loci are selected above 0.1, preferably, above 0.2, more preferable above 0.5, more preferably, above 0.6, still more preferably, above 0.7, preferably, above 0.8, more preferably above 0.9, ideally about 1.0 to 1.0.

It will be appreciated that SNPs which are present in neighboring loci but their linkage disequilibrium status with the CYP2D6*4 polymorphism is yet unknown, can be used along with the present invention. Such SNPs can be found in the genomic sequence set forth in SEQ ID NO:10.

Moreover, poor metabolizer variants of other cytochrome P450 proteins such as CYP2C19 [e.g., CYP2C19*2, *3, *4, *7, *8 (Ibeanu G C., et al., 1999; J. Pharmacol. Exp. Ther. 290: 635-40)], CYP2A6 [e.g., CYP2A6*4, T1412C (Ile471Thr, Ariyoshi, N., et al., 2001; Biochem. Biophys. Res. Commun. 281: 810-4), CgammaP2A6v1, CgammaP2A6v2, or a deletion allele of the CgammaP2A6 gene (Nakajima M., et al., 2000; Clin. Pharmacol. Ther. 67: 57-69)], CYP2C9 (e.g., CYP2C9*1, *3), CYP3A4, and CYP2E1 (e.g., CYP2E1*5B, Abdel-Rabman, S Z., et al., 2000; Pharmacogenetics 10:239-49) can be also used along with the present invention.

The predisposition to fast progression of liver fibrosis can be quantified by generating and using genotype relative risk (GRR) values. The GRR is the increased chance of an individual with a particular genotype to develop fast progression of liver fibrosis. Thus, the GRR of the risk genotype G, with respect to the protective genotype G₀, is the ratio between the risk of an individual carrying genotype G to develop fast progression of liver fibrosis, and the risk of an individual carrying genotype G₀ to develop fast progression of liver fibrosis. The GRR used herein is represented in terms of an appropriate odds ratio (OR) of G versus G₀ in cases and controls. Moreover, computation of GRR of haplotypes is based on a multiplicative model in which the GRR of an homozygote individual is the square of the GRR of an heterozygote individual. For further details see Risch and Merikangas, 1996 [The future of genetic studies of complex human diseases. Science 273: 1516-1517].

Once calculated, the GRR can reflect the increased predisposition risk on an individual with a specific CYP2D6 genotype to develop fast progression of liver fibrosis.

Fast progression of liver fibrosis can also lead to liver cirrhosis, a degenerative disease in which the parenchyma of the liver deteriorates, the lobules are infiltrated with fat and dense perilobular connective tissue are formed. The surviving cells regenerate and form “islands” of living cells with reduced blood supply. As the cirrhotic process continues, the flow of blood through the liver decreases, leading to portal hypertension, decreased liver function and eventually death.

The association of the CYP2D6*4 polymorph with an increased predisposition to development of liver fibrosis provides a tool which can be used to identify individuals predisposed to fast progression of liver fibrosis and/or cirrhosis and thus enable selection of proper treatment regimens in such individuals.

Identification of such individuals is effected by obtaining a DNA sample from the individual and testing the sample for the presence or absence of at least one fast progression of liver fibrosis-associated genotype in the CYP2D6 locus: the G/A or A/A genotype at position 3465 of the CYP2D6 gene as set forth by SEQ ID NO:6. The DNA sample can be obtained from any source of cells of the individuals, including, but not limited to, peripheral blood cells (obtained using a syringe), skin cells (obtained from a skin biopsy), mouth epithelial cells (obtained from a mouth wash), and the like. Preferably, the DNA sample is obtained from a peripheral blood sample. Methods of extracting DNA from blood samples are well known in the art.

The term “absence” as used herein in regard to the genotype describes the negative result of a specific genotype determination test. For example, if the genotype determination test is suitable for the identification of a guanine nucleotide-containing allele of the CYP2D6*4 SNP as set forth in SEQ ID NO:1, and the individual on which the test is performed is a homozygote for the adenosine nucleotide-containing allele of the CYP2D6*4 SNP, then the result of the test will be “absence of genotype”.

The fast progression of liver fibrosis-associated genotype can be identified using a variety of approaches suitable for identifying sequence alterations. One option is to determine the entire gene sequence of a PCR reaction product. Alternatively, a given segment of nucleic acid may be characterized on several other levels. At the lowest resolution, the size of the molecule can be determined by electrophoresis by comparison to a known standard run on the same gel. A more detailed picture of the molecule may be achieved by cleavage with combinations of restriction enzymes prior to electrophoresis, to allow construction of an ordered map. The presence of specific sequences within the fragment can be detected by hybridization of a labeled probe, or the precise nucleotide sequence can be determined by partial chemical degradation or by primer extension in the presence of chain-terminating nucleotide analogs.

Following is a non-limiting list of SNPs detection methods which can be used to identify one or more of the SNPs described above.

Restriction fragment length polymorphism (RFLP): This method uses a change in a single nucleotide (the SNP nucleotide) which modifies a recognition site for a restriction enzyme resulting in the creation or destruction of an RFLP.

For example, RFLP can be used to detect the CYP2D6*4 variant in a genomic DNA of an individual. Briefly, genomic DNA is amplified using the CYP2D6*4 Forward (SEQ ID NO:2) and CYP2D6*4 Reverse (SEQ ID NO:3) PCR primers, and the resultant PCR product is subjected to digestion using a restriction enzyme such as MvaI which is capable of differentially digesting a PCR product containing the G allele (and not the A allele) at position 3465 of SEQ ID NO:6.

Single nucleotide mismatches in DNA heteroduplexes are also recognized and cleaved by some chemicals, providing an alternative strategy to detect single base substitutions, generically named the “Mismatch Chemical Cleavage” (MCC) (Gogos et al., Nucl. Acids Res., 18:6807-6817, 1990). However, this method requires the use of osmium tetroxide and piperidine, two highly noxious chemicals which are not suited for use in a clinical laboratory.

Allele specific oligonucleotide (ASO): In this method, an allele-specific oligonucleotide (ASO) is designed to hybridize in proximity to the polymorphic nucleotide, such that a primer extension or ligation event can be used as the indicator of a match or a mis-match. Hybridization with radioactively labeled allelic specific oligonucleotides (ASO) also has been applied to the detection of specific SNPs (Conner et al., Proc. Natl. Acad. Sci., 80:278-282, 1983). The method is based on the differences in the melting temperature of short DNA fragments differing by a single nucleotide. Stringent hybridization and washing conditions can differentiate between mutant and wild-type alleles.

Suitable ASO probes which can be used along with the present invention to identify the presence of the CYP2D6*4 polymorphism include the 5′-AGGGGCGTCTTGGGG probe (SEQ ID NO:9) which can differentially hybridize to the CYP2D6*4 allele and the 5′-AGGGGCGTCCTGGGG probe (SEQ ID NO:8) which can differentially hybridize to the wild-type allele (i.e., CYP2D6).

Denaturing/Temperature Gradient Gel Electrophoresis (DGGE/TGGE): Two other methods rely on detecting changes in electrophoretic mobility in response to minor sequence changes. One of these methods, termed “Denaturing Gradient Gel Electrophoresis” (DGGE) is based on the observation that slightly different sequences will display different patterns of local melting when electrophoretically resolved on a gradient gel. In this manner, variants can be distinguished, as differences in melting properties of homoduplexes versus heteroduplexes differing in a single nucleotide can detect the presence of SNPs in the target sequences because of the corresponding changes in their electrophoretic mobilities. The fragments to be analyzed, usually PCR products, are “clamped” at one end by a long stretch of G-C base pairs (30-80) to allow complete denaturation of the sequence of interest without complete dissociation of the strands. The attachment of a GC “clamp” to the DNA fragments increases the fraction of mutations that can be recognized by DGGE (Abrams et al., Genomics 7:463-475, 1990). Attaching a GC clamp to one primer is critical to ensure that the amplified sequence has a low dissociation temperature (Sheffield et al., Proc. Natl. Acad. Sci., 86:232-236, 1989; and Lerman and Silverstein, Meth. Enzymol., 155:482-501, 1987). Modifications of the technique have been developed, using temperature gradients (Wartell et al., Nucl. Acids Res., 18:2699-2701, 1990), and the method can be also applied to RNA:RNA duplexes (Smith et al., Genomics 3:217-223, 1988).

Limitations on the utility of DGGE include the requirement that the denaturing conditions must be optimized for each type of DNA to be tested. Furthermore, the method requires specialized equipment to prepare the gels and maintain the needed high temperatures during electrophoresis. The expense associated with the synthesis of the clamping tail on one oligonucleotide for each sequence to be tested is also a major consideration. In addition, long running times are required for DGGE. The long running time of DGGE was shortened in a modification of DGGE called constant denaturant gel electrophoresis (CDGE) (Borrensen et al., Proc. Natl. Acad. Sci. USA 88:8405, 1991). CDGE requires that gels be performed under different denaturant conditions in order to reach high efficiency for the detection of SNPs.

A technique analogous to DGGE, termed temperature gradient gel electrophoresis (TGGE), uses a thermal gradient rather than a chemical denaturant gradient (Scholz, et al., Hum. Mol. Genet. 2:2155, 1993). TGGE requires the use of specialized equipment which can generate a temperature gradient perpendicularly oriented relative to the electrical field. TGGE can detect mutations in relatively small fragments of DNA therefore scanning of large gene segments requires the use of multiple PCR products prior to running the gel.

Single-Strand Conformation Polymorphism (SSCP): Another common method, called “Single-Strand Conformation Polymorphism” (SSCP) was developed by Hayashi, Sekya and colleagues (reviewed by Hayashi, PCR Meth. Appl., 1:34-38, 1991) and is based on the observation that single strands of nucleic acid can take on characteristic conformations in non-denaturing conditions, and these conformations influence electrophoretic mobility. The complementary strands assume sufficiently different structures that one strand may be resolved from the other. Changes in sequences within the fragment will also change the conformation, consequently altering the mobility and allowing this to be used as an assay for sequence variations (Orita, et al., Genomics 5:874-879, 1989).

The SSCP process involves denaturing a DNA segment (e.g., a PCR product) that is labeled on both strands, followed by slow electrophoretic separation on a non-denaturing polyacrylamide gel, so that intra-molecular interactions can form and not be disturbed during the run. This technique is extremely sensitive to variations in gel composition and temperature. A serious limitation of this method is the relative difficulty encountered in comparing data generated in different laboratories, under apparently similar conditions.

Dideoxy fingerprinting (ddF): The dideoxy fingerprinting (ddF) is another technique developed to scan genes for the presence of mutations (Liu and Sommer, PCR Methods Appli., 4:97, 1994). The ddF technique combines components of Sanger dideoxy sequencing with SSCP. A dideoxy sequencing reaction is performed using one dideoxy terminator and then the reaction products are electrophoresed on nondenaturing polyacrylamide gels to detect alterations in mobility of the termination segments as in SSCP analysis. While ddF is an improvement over SSCP in terms of increased sensitivity, ddF requires the use of expensive dideoxynucleotides and this technique is still limited to the analysis of fragments of the size suitable for SSCP (i.e., fragments of 200-300 bases for optimal detection of mutations).

In addition to the above limitations, all of these methods are limited as to the size of the nucleic acid fragment that can be analyzed. For the direct sequencing approach, sequences of greater than 600 base pairs require cloning, with the consequent delays and expense of either deletion sub-cloning or primer walking, in order to cover the entire fragment. SSCP and DGGE have even more severe size limitations. Because of reduced sensitivity to sequence changes, these methods are not considered suitable for larger fragments. Although SSCP is reportedly able to detect 90% of single-base substitutions within a 200 base-pair fragment, the detection drops to less than 50% for 400 base pair fragments. Similarly, the sensitivity of DGGE decreases as the length of the fragment reaches 500 base-pairs. The ddF technique, as a combination of direct sequencing and SSCP, is also limited by the relatively small size of the DNA that can be screened.

Pyrosequencing™ analysis (Pyrosequencing, Inc. Westborough, Mass., USA): This technique is based on the hybridization of a sequencing primer to a single stranded, PCR-amplified, DNA template in the presence of DNA polymerase, ATP sulfurylase, luciferase and apyrase enzymes and the adenosine 5′ phosphosulfate (APS) and luciferin substrates. In the second step the first of four deoxynucleotide triphosphates (dNTP) is added to the reaction and the DNA polymerase catalyzes the incorporation of the deoxynucleotide triphosphate into the DNA strand, if it is complementary to the base in the template strand. Each incorporation event is accompanied by release of pyrophosphate (PPi) in a quantity equimolar to the amount of incorporated nucleotide. In the last step the ATP sulfurylase quantitatively converts PPi to ATP in the presence of adenosine 5′ phosphosulfate. This ATP drives the luciferase-mediated conversion of luciferin to oxyluciferin that generates visible light in amounts that are proportional to the amount of ATP. The light produced in the luciferase-catalyzed reaction is detected by a charge coupled device (CCD) camera and seen as a peak in a Pyrogram™. Each light signal is proportional to the number of nucleotides incorporated.

Acycloprime™ analysis (Perkin Elmer, Boston, Mass., USA): This technique is based on fluorescent polarization (FP) detection. Following PCR amplification of the sequence containing the SNP of interest, excess primer and dNTPs are removed through incubation with shrimp alkaline phosphatase (SAP) and exonuclease I. Once the enzymes are heat inactivated, the Acycloprime-FP process uses a thermostable polymerase to add one of two fluorescent terminators to a primer that ends immediately upstream of the SNP site. The terminator(s) added are identified by their increased FP and represent the allele(s) present in the original DNA sample. The Acycloprime process uses AcycloPol™, a novel mutant thermostable polymerase from the Archeon family, and a pair of AcycloTerminators™ labeled with R110 and TAMRA, representing the possible alleles for the SNP of interest. AcycloTerminator™ non-nucleotide analogs are biologically active with a variety of DNA polymerases. Similarly to 2′,3′-dideoxynucleotide-5′-triphosphates, the acyclic analogs function as chain terminators. The analog is incorporated by the DNA polymerase in a base-specific manner onto the 3′-end of the DNA chain, and since there is no 3′-hydroxyl, is unable to function in further chain elongation. It has been found that AcycloPol has a higher affinity and specificity for derivatized AcycloTerminators than various Taq mutant have for derivatized 2′,3′-dideoxynucleotide terminators.

Reverse dot blot: This technique uses labeled sequence specific oligonucleotide probes and unlabeled nucleic acid samples. Activated primary amine-conjugated oligonucleotides are covalently attached to carboxylated nylon membranes. After hybridization and washing, the labeled probe, or a labeled fragment of the probe, can be released using oligomer restriction, i.e., the digestion of the duplex hybrid with a restriction enzyme. Circular spots or lines are visualized colorimetrically after hybridization through the use of streptavidin horseradish peroxidase incubation followed by development using tetramethylbenzidine and hydrogen peroxide, or via chemiluminescence after incubation with avidin alkaline phosphatase conjugate and a luminous substrate susceptible to enzyme activation, such as CSPD, followed by exposure to x-ray film.

LightCycler™ Analysis (Roche, Indianapolis, Ind., USA)—The LightCycler™ instrument consists of a thermocycler and a fluorimeter component for on-line detection. PCR-products formed by amplification are detected on-line through fluorophores coupled to two sequence-specific oligonucleotide hybridization probes. One of the oligonucleotides has a fluorescein label at its 3′-end (donor oligonucleotide) and the other oligonucleotide is labeled with LightCyler™-Red 640 at its 5′-end (acceptor oligonucleotide). When both labeled DNA-probes are hybridized to their template, energy is transferred from the donor fluorophore to the acceptor fluorophore following the excitation of the donor fluorophore using an external light source with a specific wavelength. The light that is emitted by the acceptor fluorophore can be detected at a defined wavelength. The intensity of this light signal is proportional to the amount of PCR-product.

For example, as is shown in Example 1 of the Examples section which follows, the CYP2D6*4 Forward and CYP2D6*4 Reverse PCR primers (SEQ ID NOs:2 and 3, respectively) were used to amplify a 347 bp PCR product which was further analyzed by the LightCycler™ using the Anchor and Mutation probes (SEQ ID NOs:7 and 8, respectively) analysis to detect the presence of the CYP2D6*4 polymorphism.

It will be appreciated that advances in the field of SNP detection have provided additional accurate, easy, and inexpensive large-scale SNP genotyping techniques, such as dynamic allele-specific hybridization (DASH, Howell, W. M. et al., 1999. Dynamic allele-specific hybridization (DASH). Nat. Biotechnol. 17: 87-8), microplate array diagonal gel electrophoresis [MADGE, Day, I. N. et al., 1995. High-throughput genotyping using horizontal polyacrylamide gels with wells arranged for microplate array diagonal gel electrophoresis (MADGE). Biotechniques. 19: 830-5], the TaqMan system (Holland, P. M. et al., 1991. Detection of specific polymerase chain reaction product by utilizing the 5′→3′ exonuclease activity of Thermus aquaticus DNA polymerase. Proc Natl Acad Sci USA. 88: 7276-80), as well as various DNA “chip” technologies such as the GeneChip microarrays (e.g., Affymetrix SNP chips) which are disclosed in U.S. Pat. No. 6,300,063 to Lipshutz, et al. 2001, which is fully incorporated herein by reference, Genetic Bit Analysis (GBA™) which is described by Goelet, P. et al. (PCT Appl. No. 92/15712), peptide nucleic acid (PNA, Ren B, et al., 2004. Nucleic Acids Res. 32: e42) and locked nucleic acids (LNA, Latorra D, et al., 2003. Hum. Mutat. 22: 79-85) probes, Molecular Beacons (Abravaya K, et al., 2003. Clin Chem Lab Med. 41: 468-74), intercalating dye [Germer, S. and Higuchi, R. Single-tube genotyping without oligonucleotide probes. Genome Res. 9:72-78 (1999)], FRET primers (Solinas A et al., 2001. Nucleic Acids Res. 29: E96), AlphaScreen (Beaudet L, et al., Genome Res. 2001, 11(4): 600-8), SNPstream (Bell P A, et al., 2002. Biotechniques. Suppl.: 70-2, 74, 76-7), Multiplex minisequencing (Curcio M, et al., 2002. Electrophoresis. 23: 1467-72), SnaPshot (Turner D, et al., 2002. Hum Immunol. 63: 508-13), MassEXTEND (Cashman J R, et al., 2001. Drug Metab Dispos. 29: 1629-37), GOOD assay (Sauer S, and Gut I G. 2003. Rapid Commun. Mass. Spectrom. 17: 1265-72), Microarray minisequencing (Liljedahl U, et al., 2003. Pharmacogenetics. 13: 7-17), arrayed primer extension (APEX) (Tonisson N, et al., 2000. Clin. Chem. Lab. Med. 38: 165-70), Microarray primer extension (O'Meara D, et al., 2002. Nucleic Acids Res. 30: e75), Tag arrays (Fan J B, et al., 2000. Genome Res. 10: 853-60), Template-directed incorporation (TDI) (Akula N, et al., 2002. Biotechniques. 32: 1072-8), fluorescence polarization (Hsu T M, et al., 2001. Biotechniques. 31: 560, 562, 564-8), Colorimetric oligonucleotide ligation assay (OLA, Nickerson D A, et al., 1990. Proc. Natl. Acad. Sci. USA. 87: 8923-7), Sequence-coded OLA (Gasparini P, et al., 1999. J. Med. Screen. 6: 67-9), Microarray ligation, Ligase chain reaction, Padlock probes, Rolling circle amplification, Invader assay (reviewed in Shi M M. 2001. Enabling large-scale pharmacogenetic studies by high-throughput mutation detection and genotyping technologies. Clin Chem. 47: 164-72), coded microspheres (Rao K V et al., 2003. Nucleic Acids Res. 31: e66) and MassArray (Leushner J, Chiu N H, 2000. Mol. Diagn. 5: 341-80).

It will be appreciated that genetic polymorphisms which occur in the coding sequence of a protein and result in a change of the protein sequence may be detected directly, by analyzing the protein gene product of CYP2D6, or portions thereof. Non-limiting examples of such genetic polymorphism include a missense mutation (i.e., substitution of an amino acid), a non-sense mutation (i.e., introduction of a stop codon instead of an amino acid), a deletion (i.e., deletion of at least one amino acid), a duplication and/or insertion (i.e., insertion of additional amino acids) and a splice mutation which can result in exclusion or inclusion of coding (i.e., exons) or non-coding (i.e., introns) sequences, respectively. For example, the 3465G→A splice mutation in the CYP2D6 gene (SEQ ID NO:6) results in a truncated protein as a result of an exclusion of a coding sequence. The direct analysis of protein gene product of CYP2D6, or portions thereof may be accomplished using an immunological detection method.

Immunological detection methods: The immunological detection methods used in context of the present invention are fully explained in, for example, “Using Antibodies: A Laboratory Manual” [Ed Harlow, David Lane eds., Cold Spring Harbor Laboratory Press (1999)] and those familiar with the art will be capable of implementing the various techniques summarized hereinbelow as part of the present invention. All of the immunological techniques require antibodies specific to at least one of the CYP2D6 alleles. Immunological detection methods suited for use as part of the present invention include, but are not limited to, radio-immunoassay (RIA), enzyme linked immunosorbent assay (ELISA), western blot, immunohistochemical analysis, and fluorescence activated cell sorting (FACS).

Radio-immunoassay (RIA): In one version, this method involves precipitation of the desired substrate, CYP2D6 in this case, with a specific antibody and radiolabelled antibody binding protein (e.g., protein A labeled with I¹²⁵) immobilized on a precipitable carrier such as agarose beads. The number of counts in the precipitated pellet is proportional to the amount of substrate.

In an alternate version of the RIA, a labeled substrate and an unlabelled antibody binding protein are employed. A sample containing an unknown amount of substrate is added in varying amounts. The decrease in precipitated counts from the labeled substrate is proportional to the amount of substrate in the added sample.

Enzyme linked immunosorbent assay (ELISA): This method involves fixation of a sample (e.g., fixed cells or a proteinaceous solution) containing a protein substrate to a surface such as a well of a microtiter plate. A substrate specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western blot: This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabelled or enzyme linked as described hereinabove. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis.

Immunohistochemical analysis: This method involves detection of a substrate in situ in fixed cells by substrate specific antibodies. The substrate specific antibodies may be enzyme linked or linked to fluorophores. Detection is by microscopy and subjective evaluation. If enzyme linked antibodies are employed, a colorimetric reaction may be required.

Fluorescence activated cell sorting (FACS): This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

It will be appreciated by one ordinarily skilled in the art that determining the CYP2D6 phenotype of an individual, either directly (e.g., by detecting the protein polymorphs) or genetically (e.g., by detecting the presence or absence of SNP genotypes), may be effected using any suitable biological sample derived from the examined individual, including, but not limited to, blood, plasma, blood cells, saliva or cells derived by mouth wash, and body secretions such as urine and tears, and from biopsies, etc. Alternatively, nucleic acid tests can be performed on dry samples (e.g. hair or skin). The sample may contain genomic DNA, cDNA or RNA. Methods of preparing genomic DNA or cDNA and RNA are well known in the art.

The antibody used in the method of the present invention is selected differentially interactable with at least one form of a CYP2D6 protein encoded by a CYP2D6*4 polymorphism and can differentiate between the wild-type protein (i.e., CYP2D6) and the poor metabolizer polymorph (e.g., CYP2D6*3, CYP2D6*4, CYP2D6*5) via differential antibody interaction. Antibodies useful in context of this embodiment of the invention can be prepared using methods of antibody preparation well known to one of ordinary skills in the art, using, for example, synthetic peptides derived from the various domains of the CYP2D6 protein for vaccination of antibody producing animals and subsequent isolation of antibodies therefrom. Monoclonal antibodies specific to each of the CYP2D6 variants can also be prepared as is described, for example, in “Current Protocols in Immunology” Volumes I-III Coligan J. E., Ed. (1994); Stites et al. (Eds), “Basic and Clinical Immunology” (8th Edition), Appleton & Lange, Norwalk, Conn. (1994); Mishell and Shiigi (Eds), “Selected Methods in Cellular Immunology”, W. H. Freeman and Co., New York (1980).

The term “antibody” as used in the present invention includes intact molecules as well as functional fragments thereof, such as Fab, F(ab′)₂, and Fv that are capable of binding to macrophages. These functional antibody fragments are defined as follows: Fab, the fragment which contains a monovalent antigen-binding fragment of an antibody molecule, can be produced by digestion of whole antibody with the enzyme papain to yield an intact light chain and a portion of one heavy chain; Fab′, the fragment of an antibody molecule that can be obtained by treating whole antibody with pepsin, followed by reduction, to yield an intact light chain and a portion of the heavy chain; two Fab′ fragments are obtained per antibody molecule; (Fab′)₂, the fragment of the antibody that can be obtained by treating whole antibody with the enzyme pepsin without subsequent reduction; F(ab′)₂ is a dimer of two Fab′ fragments held together by two disulfide bonds; Fv, defined as a genetically engineered fragment containing the variable region of the light chain and the variable region of the heavy chain expressed as two chains; and single chain antibody (“SCA”), a genetically engineered molecule containing the variable region of the light chain and the variable region of the heavy chain, linked by a suitable polypeptide linker as a genetically fused single chain molecule.

Methods of making these fragments are known in the art. See for example, Harlow and Lane, Antibodies: A Laboratory Manual, Cold Spring Harbor Laboratory, New York, 1988, incorporated herein by reference.

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment.

Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R., Biochem. J., 73: 119-126, 1959. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of V_{H} and V_{L} chains. This association may be noncovalent, as described in Inbar et al., Proc. Nat'l Acad. Sci. USA 69:2659-62, 1972. Alternatively, the variable chains can be linked by an intermolecular disulfide bond or cross-linked by chemicals such as gluteraldehyde. Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow and Filpula, Methods, 2: 97-105, 1991; Bird et al., Science 242:423-426, 1988; Pack et al., Bio/Technology 11:1271-77, 1993; and Ladner et al., U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry, Methods, 2: 106-10, 1991.

It will be appreciated that the reagents utilized by the methods for determining predisposition to fast progression of liver fibrosis according to the present invention and which are described hereinabove can form a part of a kit.

Such a kit includes at least one reagent for determining a presence or absence in a homozygous or heterozygous form, of at least one fast progression liver fibrosis-associated genotype in the CYP2D6 locus or in neighboring loci which are in linkage disequilibrium with the CYP2D6 locus.

According to preferred embodiments the kit further includes packaging material and a notification in or on the packaging material identifying the kit for use in determining if an individual is predisposed to fast progression of liver fibrosis.

The kit also includes the appropriate instructions for use and labels indicating FDA approval for use in diagnostics.

The methods and kits of determining predisposition of an individual to develop fast progression of liver fibrosis according to the present invention can be used to determine suitability of individuals infected with HCV, hepatitis B virus (HBV), non-alcoholic steatohepatitis (NASH) to antiviral or other therapy. This is of particular importance since such a treatment using a combination of PEG-interferon and Ribavirin is not been offered to all HCV-infected individuals. As a result, in some cases the antiviral treatment is employed on individuals which are unlikely to develop liver fibrosis in their life-time and, more importantly, in other cases, antiviral therapy is withheld (due to budget limitations) from individuals which are at risk of developing fast progression of liver fibrosis but are mis-diagnosed.

Following is a list of clinical situations in which the method of the present invention can be used to assist in determining suitability to antiviral treatment.

HCV-infected patients with normal ALT/AST levels, and no fibrosis on histology. This group of patients, consisting of 25% of HCV patients (Hepatology 1998; 27:1213), can be followed with liver biopsies at 5-year intervals to assess the extent of progression if any, since approximately 80% of them will not progress significantly towards fibrosis (Gastroenterology 2004; 126: 1409). Knowing the predisposiotn risk of such individuals to develop fast progression of liver fibrosis can assist, for example, in determining the intervals in which liver biopsy should be performed.

Fifty percent of the HCV-infected individuals with HCV genotype type 1 are complete non-responders to combination therapy, i.e., the combination therapy fails to decrease HCV RNA levels by 2 logs following 4, 12, or 24 weeks of treatment (J. Hepatol. 1999; 30: 192-198, Hepatology 2003; 38: 248A, Hepatology 2003; 38: 208A). In addition, fifteen percent of the HCV-infected individuals with HCV genotype type 1 are partial-responders following 24 weeks of combination therapy, i.e., they have a decreased level of HCV RNA of more than 2 logs but have not cleared the HCV virus at 24 weeks (Hepatology 2003; 38: 645-652). Such patients have a reduced likelihood of achieving a sustained viral response at 48 weeks. A recent study has shown that 18 months of treatment is likely to result in higher success than 12 months of treatment. However, such a long period of treatment is not widely accepted due to cost effectiveness (Reduction of relapse rates by 18-month treatment in chronic hepatitis C. A Benelux randomized trial in 300 patients. J Hepatol. 2004; 40(4):689-695). Thus, while nonresponders or partial responders who are slow fibrosers can wait for a better treatment to be developed, fast fibrosers who are partial or complete non-responders should be subjected for a longer duration (i.e., 18 months) of the currently available antiviral treatment.

HCV-infected individuals with HCV genotype type 1 and elevated AST/ALT, but normal abdominal ultrasound, who refuse a liver biopsy. In this case, if such patients are predisposed to fast progression of liver fibrosis they should be treated even in the absence of a biopsy.

HCV-infected patients with decompensated cirrhosis (<10% of the HCV-infected individuals in Israel) are usually considered for liver transplantation and not the antiviral therapy. However, in many cases, the transplanted liver is also subjected to liver fibrosis and cirrhosis. In such cases, determination of increased predisposition risk to develop fast progression of liver fibrosis can be used to anticipate the success or failure of liver transplantation.

HCV patients at an age older than 70 years (approximately 5% of the total HCV-infected individuals), especially if they have one or more other life threatening medical conditions are usually not being offered the PEG interferon, but regular interferon. However, if such individuals are predisposed to fast progression of liver fibrosis they should be considered for treatment as well.

HCV-infected individuals with significant obesity (BMI >30 Kg/sq m) have a reduced response rate to therapy. Such patient should undergo a strict weight reduction program before PEG interferon therapy is considered (Hepatology 2003; 38: 639). In this case, if such patients are predisposed to fast progression of liver fibrosis they should be counseled regarding this life-threatening situation and be motivated to loose a considerable amount of weight.

HCV-infected individuals who are drinking a significant amount of alcohol daily, i.e. >2 units of alcohol per day, and exhibit elevated levels of serum ALT/AST are advised to stop drinking prior to the administration of antiviral therapy. This group of patients comprises <5% of the Israeli HCV-infected individuals. The predisposition risk to develop fast progression of liver fibrosis together with the level of ALT/AST in the serum as detected six months following cessation of alcohol intake may be taken into consideration prior to the administration of the combination of antiviral therapy.

Some HCV-infected individuals exhibit an iron overload, i.e., excessive iron on liver biopsy associated with elevated body iron storage markers. Such patients are offered monthly venesection therapy until a significant fall in hemoglobin levels occurs. If serum ALT/AST levels normalizes, these patients are usually managed as the HCV Group 1 patients. If the enzymes level remains elevated, these patients are usually considered for a combination of antiviral therapy. In these cases the knowledge of the predisposition risk to develop fast progression of liver fibrosis may affect the choice of treatment.

HCV-infected individuals which present with thrombocytopenia, i.e., with a platelet count of less than 50,000, are not being offered with the combination of antiviral therapy. However, if such patients are predisposed to fast progression of liver fibrosis they can be treated with factors such as G-CSF, that are not routinely offered from cost effective point of view, as well as Ribavirin only.

HCV-infected individuals with a history of depression requiring the use of anti-depressive therapy, with or without suicidal attempts, are usually not being offered with the of antiviral therapy. In such cases, if the patients are not predisposed to fast progression of liver fibrosis (i.e., they are slow fibrosers) they should be followed-up periodically with no treatment.

In addition, the predisposition status of an individual to develop fast progression of liver fibrosis can be also used in genetic counseling, providing the individual with recommended guidelines which might prevent and/or delay the onset of liver fibrosis and/or cirrhosis. For example, an individual infected with HCV which is predisposed to fast progression of liver fibrosis should avoid any alcohol consumption, decrease fat intake and increase physical activity.

It will be appreciated that since the poor metabolizer form of CYP2D6 (CYP2D6*4) is associated with increased predisposition risk to develop fast progression of liver fibrosis and/or cirrhosis upregulation thereof can be utilized to prevent the fast progression of liver fibrosis.

Thus, according to another aspect of the present invention there is provided a method of preventing fast progression of liver fibrosis in an individual in need thereof.

The term “preventing” as used herein refers to avoiding the progression of liver fibrosis and/or delaying the onset of liver fibrosis.

As used herein, the phrase “an individual in need thereof” refers to any individual as described hereinabove which is likely to develop fast progression of liver fibrosis. It will be appreciated that the phrase “an individual in need thereof” encompasses also an individual which is identified as predisposed to fast progression according to the teachings of the present invention.

The method is effected by administering to the individual an agent capable of upregulating the expression level and/or activity of CYP2D6 in the liver of the individual, thereby preventing fast progression of liver fibrosis in the individual.

The term “upregulating” as used herein refers to increasing the expression and/or activity of CYP2D6.

Upregulation of CYP2D6 can be effected at the genomic level (i.e., activation of transcription via promoters, enhancers, regulatory elements), at the transcript level (i.e., correct splicing, polyadenylation, activation of translation) or at the protein level (i.e., post-translational modifications, interaction with substrates and the like).

Following is a list of agents capable of upregulating the expression level and/or activity of CYP2D6.

An agent capable of upregulating expression level of a CYP2D6 may be an exogenous polynucleotide sequence designed and constructed to express at least a functional portion of the CYP2D6 protein. Accordingly, the exogenous polynucleotide sequence may be a DNA or RNA sequence encoding a CYP2D6 molecule, which is capable of metabolizing a variety of drugs such as debrisoquine, sparteine, propafenone, and amitryptiline.

The phrase “functional portion” as used herein refers to part of the CYP2D6 protein (i.e., a polypeptide) which exhibits functional properties of the enzyme such as binding or degrading the substrate. According to preferred embodiments of the present invention the functional portion of CYP2D6 is a polypeptide sequence including amino acids 58-493 (region of cytochrome P450) as set forth in SEQ ID NO:4. Preferably, the functional portion of CYP2D6 is a polypeptide sequence including amino acids 58-497, more preferably, amino acids 1-497 as set forth in SEQ ID NO:4.

CYP2D6 has been cloned from human and Bos taurus sources. Thus, coding sequences information for CYP2D6 is available from several databases including the GenBank database available through http://www.ncbi.nlm.nih.gov/.

To express exogenous CYP2D6 in mammalian cells, a polynucleotide sequence encoding a CYP2D6 (GenBank Accession number NM_{—}000106, SEQ ID NO:5) is preferably ligated into a nucleic acid construct suitable for mammalian cell expression. Such a nucleic acid construct includes a promoter sequence for directing transcription of the polynucleotide sequence in the cell in a constitutive or inducible manner.

It will be appreciated that the nucleic acid construct of the present invention can also utilize CYP2D6 homologues which exhibit the desired activity (i.e., drug metabolism). Such homologues can be, for example, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to SEQ ID NO:5, as determined using the BestFit software of the Wisconsin sequence analysis package, utilizing the Smith and Waterman algorithm, where gap weight equals 50, length weight equals 3, average match equals 10 and average mismatch equals −9.

Constitutive promoters suitable for use with the present invention are promoter sequences which are active under most environmental conditions and most types of cells such as the cytomegalovirus (CMV) and Rous sarcoma virus (RSV). Inducible promoters suitable for use with the present invention include for example tetracycline-inducible promoter (Zabala M, et al., Cancer Res. 2004, 64(8): 2799-804). It will be appreciated that a dual system comprising a responsive promoter driving expression of the polynucleotide encoding CYP2D6 and a ligand-inducible chimeric transcription factor containing a novel ligand binding site can be also used in order to express the CYP2D6 protein in liver cells (for further details see Zerby D et al., Hum Gene Ther. 2003; 14: 749-61).

The nucleic acid construct (also referred to herein as an “expression vector”) of the present invention includes additional sequences which render this vector suitable for replication and integration in prokaryotes, eukaryotes, or preferably both (e.g., shuttle vectors). In addition, a typical cloning vector may also contain a transcription and translation initiation sequence, transcription and translation terminator and a polyadenylation signal.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for the present invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of CYP2D6 mRNA translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for the present invention include those derived from SV40.

In addition to the elements already described, the expression vector of the present invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

The expression vector of the present invention can further include additional polynucleotide sequences that allow, for example, the translation of several proteins from a single mRNA such as an internal ribosome entry site (IRES) and sequences for genomic integration of the promoter-chimeric polypeptide.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A⁺, pMTO10/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by the present invention will depend on the cell type transformed. The ability to select suitable vectors according to the cell type transformed is well within the capabilities of the ordinary skilled artisan and as such no general description of selection consideration is provided herein. For example, liver cells can be targeted using the human gutless adenoviral vector system as described in Zerby D et al., Hum Gene Ther. 2003; 14(8):749-61.

Recombinant viral vectors are useful for in vivo expression of CYP2D6 since they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

Various methods can be used to introduce the expression vector of the present invention into stem cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Introduction of nucleic acids by viral infection offers several advantages over other methods such as lipofection and electroporation, since higher transfection efficiency can be obtained due to the infectious nature of viruses.

For example, recombinant E1-deleted adenoviral vectors containing the antisense sequence of TGF-β1 gene under the control of human CMV promoter were shown to prevent liver fibrosis in bile-duct ligated rats (Arias, M., et al., 2003. BMC Gastroenterol. 3: 29).

It will be appreciated that upregulation of CYP2D6 can be also effected by administration of CYP2D6-expressing cells into the liver of the individual.

CYP2D6-expressing cells can be any suitable cells, such as hepatic cells and bone marrow cells which are derived from the individuals and are transfected ex vivo with an expression vector containing the polynucleotide designed to express CYP2D6 as described hereinabove.

Administration of the CYP2D6-expressing cells of the present invention can be effected using any suitable route such as intravenous, intraportal, intra peritoneal, intra liver, intra gastrointestinal track, intrasplenic, subcapsular of any other organ, and the like. According to presently preferred embodiments, the CYP2D6-expressing cells of the present invention are introduced to the individual using intravenous, intra liver, intra gastrointestinal track and/or intra peritoneal administrations.

CYP2D6-expressing cells of the present invention can be derived from either autologous sources such as self bone marrow or hepatic cells or from allogeneic sources such as bone marrow or hepatic cells derived from non-autologous sources. Since non-autologous cells are likely to induce an immune reaction when administered to the body several approaches have been developed to reduce the likelihood of rejection of non-autologous cells. These include either suppressing the recipient immune system or encapsulating the non-autologous cells or tissues in immunoisolating, semipermeable membranes before transplantation.

Encapsulation techniques are generally classified as microencapsulation, involving small spherical vehicles and macroencapsulation, involving larger flat-sheet and hollow-fiber membranes (Uludag, H. et al. Technology of mammalian cell encapsulation. Adv Drug Deliv Rev. 2000; 42: 29-64).

Methods of preparing microcapsules are known in the arts and include for example those disclosed by Lu M Z, et al., Cell encapsulation with alginate and alpha-phenoxycinnamylidene-acetylated poly(allylamine). Biotechnol Bioeng. 2000, 70: 479-83, Chang T M and Prakash S. Procedures for microencapsulation of enzymes, cells and genetically engineered microorganisms. Mol. Biotechnol. 2001, 17: 249-60, and Lu M Z, et al., A novel cell encapsulation method using photosensitive poly(allylamine alpha-cyanocinnamylideneacetate). J Microencapsul. 2000, 17: 245-51.

For example, microcapsules are prepared by complexing modified collagen with a ter-polymer shell of 2-hydroxyethyl methylacrylate (HEMA), methacrylic acid (MAA) and methyl methacrylate (MMA), resulting in a capsule thickness of 2-5 μm. Such microcapsules can be further encapsulated with additional 2-5 μm ter-polymer shells in order to impart a negatively charged smooth surface and to minimize plasma protein absorption (Chia, S. M. et al. Multi-layered microcapsules for cell encapsulation Biomaterials. 2002 23: 849-56).

Other microcapsules are based on alginate, a marine polysaccharide (Sambanis, A. Encapsulated islets in diabetes treatment. Diabetes Thechnol. Ther. 2003, 5: 665-8) or its derivatives. For example, microcapsules can be prepared by the polyelectrolyte complexation between the polyanions sodium alginate and sodium cellulose sulphate with the polycation poly(methylene-co-guanidine) hydrochloride in the presence of calcium chloride.

It will be appreciated that cell encapsulation is improved when smaller capsules are used. Thus, the quality control, mechanical stability, diffusion properties, and in vitro activities of encapsulated cells improved when the capsule size was reduced from 1 mm to 400 μm (Canaple L. et al., Improving cell encapsulation through size control. J Biomater Sci Polym Ed. 2002; 13: 783-96). Moreover, nanoporous biocapsules with well-controlled pore size as small as 7 nm, tailored surface chemistries and precise microarchitectures were found to successfully immunoisolate microenvironments for cells (Williams D. Small is beautiful: microparticle and nanoparticle technology in medical devices. Med Device Technol. 1999, 10: 6-9; Desai, T. A. Microfabrication technology for pancreatic cell encapsulation. Expert Opin Biol Ther. 2002, 2: 633-46).

An agent capable of upregulating a CYP2D6 expression in the liver may be any compound which is capable of increasing the transcription and/or translation of an endogenous DNA or mRNA encoding the CYP2D6 in the liver.

An agent capable of upregulating CYP2D6 activity in the liver may be an exogenous polypeptide including at least a functional portion (as described hereinabove) of the CYP2D6. According to preferred embodiments of the present invention such a polypeptide is at least 75%, at least 80%, at least 85%, more preferably, at least 88%, at least 90%, more preferably, at least 95%, most preferably, at least 99% identical to the polypeptide set forth by SEQ ID NO:4 as determined using the BlastP software of the National Center of Biotechnology Information (NCBI) using default parameters.

According to presently preferred embodiments the CYP2D6 polypeptide is set forth by SEQ ID NO:4.

It will be appreciated that agents which are capable of upregulating CYP2D6 expression level and/or activity can be also used in preventing liver cirrhosis in individuals suffering from a disease such as chronic HCV, hepatotoxic viral infection (e.g., hepatitis B, D), liver cancer, hepatotoxic alcohol or drugs, non alcoholic fatty liver disease (NAFLD), autoimmune diseases such as autoimmune hepatitis (AIH), primary biliari cirrhosis (PBC) and primary sclerosing cholangitis (PSC), metabolic liver disease such as Hemochromatosis, Wilson's disease and alpha 1 anti trypsin and diseases with secondary involvement of the liver like celiac disease or amyloidosis.

Each of the upregulating agents described hereinabove or the expression vector encoding CYP2D6 can be administered to the individual per se or as part of a pharmaceutical composition which also includes a physiologically acceptable carrier. The purpose of a pharmaceutical composition is to facilitate administration of the active ingredient to an organism.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the upregulating agent or the expression vector encoding CYP2D6 which are accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intravenous, intraperitoneal, intranasal, or intraocular injections.

Alternately, one may administer the pharmaceutical composition in a local rather than systemic manner, for example, via injection of the pharmaceutical composition directly into a tissue region of a patient.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (the upregulating agent or the expression vector encoding CYP2D6) effective to prevent, alleviate or ameliorate symptoms of a disorder (e.g., fast progression of liver fibrosis and/or liver cirrhosis) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition (See e.g., Fingl, et al., 1975, in “The Pharmacological Basis of Therapeutics”, Ch. 1 p. 1).

Dosage amount and interval may be adjusted individually to provide plasma levels of the active ingredient are sufficient to prevent fast progression of liver fibrosis and/or cirrhosis (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as if further detailed above.

Methods of evaluating upregulation of CYP2D6 in cells of the individual are known in the art and include both immunological detection methods (as described hereinabove), cytochemical methods (e.g., in situ activity assay and in vitro activity assays) and molecular methods such as Northern Blot hybridization, RT-PCR analysis, RNA in situ hybridization stain, in situ RT-PCR stain (Nuovo G J, et al. Am J Surg Pathol. 1993, 17: 683-90; Komminoth P, et al. Pathol Res Pract. 1994, 190: 1017-25).

Thus, the teachings of the present invention can be used to prevent fast progression of liver fibrosis and/or cirrhosis in individuals suffering from chronic hepatitis C. For example, an expression vector (e.g., a viral vector) including a polynucleotide sequence encoding the CYP2D6 mRNA (SEQ ID NO:5) and the suitable promoter sequences to enable expression in liver cells is introduced into the individual via intravenous or intra-hepatic administration. Expression of such a vector in the liver is expected to upregulate the expression level and/or activity of CYP2D6 in the liver and thus to prevent fast progression of liver fibrosis and/or liver cirrhosis. Dosage of such an expression vector should be calibrated using cell culture experiments and animal models. Success of treatment is preferably evaluated by subjecting the individual to a CYP2D6 substrate (e.g., debrisoquine) and determining the plasma level of its metabolites before and after treatment, essentially as described elsewhere (Rodriguez C A et al., 2004. J Clin Pharmacol. 44: 276-83).

It will be appreciated, that if such a treatment is employed shortly after infection with the HCV, i.e., prior to the appearance of any signs of liver fibrosis, it may prevent the progression of liver fibrosis in the individual. In addition, since the expression vector is targeted to somatic cells which exhibit limited half-life (depending upon the cell line transduced), such a treatment is expected to be repeated periodically in order to prevent liver fibrosis or fast progression of liver fibrosis and/or cirrhosis.

As is mentioned before, CYP2D6 is involved in the metabolism of over 50 clinically important drugs. Thus, in the presence of a poor metabolizer form of CYP2D6 (e.g., CYP2D6*4) some of these drug molecules (i.e., CYP2D6 targets such as metoprolol, propanolol, encamide, codeine, clozapine, dextromethorphan, haloperidol, amitriptyline, imipramine and sparteine) are expected to accumulate in the body of the individual and may contribute to the acceleration of liver fibrosis.

In order to identify which drug molecules may accelerate liver fibrosis there is a need to identify which drug's metabolism rate is reduced in the presence of the poor metabolizer form of CYP2D6 (e.g., CYP2D6*4) as compared with the wild-type form.

Thus, the present invention also contemplates a method of determining if a drug molecule is capable of inducing or accelerating development of fast progression of liver fibrosis in an individual.

The method is effected by comparing a metabolism rate of the drug molecule by a CYP2D6 and a poor metabolizing variant of the CYP2D6, wherein poor metabolism of the drug molecule by the poor metabolizing variant of the CYP2D6 and not the CYP2D6 is indicative of its capability of inducing or accelerating development of fast progression of liver fibrosis in the individual.

As used the phrase “poor metabolizing variant of the CYP2D6” refers to any CYP2D6 variant or a polynucleotide expressing at least a functional portion of a CYP2D6 variant which exhibits poor metabolizing activity of a specific substrate. Non-limiting examples of such variants are the CYP2D6*3, CYP2D6*4 and CYP2D6*6. Preferably, the poor metabolizing variant of CYP2D6 used by the present invention is CYP2D6*4, or a polynucleotide expressing same.

The rate of drug metabolism can be detected by measuring the accumulation of the drug's metabolites in vitro using for example, microsome preparations of in vitro expression systems derived from a cell line such as human lymphoblastoid cell line. In these systems the wild-type CYP2D6 or the poor metabolizer variant thereof (e.g., CYP2D6*4) can be applied along with the candidate drug molecule and the appropriate incubation buffer, and the rate of drug metabolism can be detected (see for example, Goto A et al., 2004. Identification of human p450 isoforms involved in the metabolism of the antiallergic drug, oxatomide, and its inhibitory effect on enzyme activity. Biol. Pharm. Bull. 27: 684-90).

Additionally, the rate of drug metabolism can be measured ex vivo using, for example, human liver microsomes, essentially as described in Wojcikowski J et al., 2004 (The metabolism of the piperazine-type phenothiazine neuroleptic perazine by the human cytochrome P-450 isoenzymes. Eur. Neuropsychopharmacol. 14:199-208).

Methods of preparing human liver microsomes are known in the art and include for example, those described in Nelson et al., 2001 (Drug Metab Dispos. 29: 319-25). Briefly, a piece of a liver tissue (about 10 g) obtained from liver biopsy is minced with scissors and is further homogenized using 10 strokes, 15 seconds each, of a Teflon-glass homogenizer (870 rpm) in a 25 ml of ice-chilled homogenization buffer (0.1 M potassium phosphate buffer, pH 7.4, containing 0.125 M potassium chloride and 1.0 mM EDTA). Homogenates are diluted to 4 volumes of sample weight (approximately 40 ml) and centrifuged for 20 min at 12,000 g in a Sorvall RC-5B using a Sorvall SA-600 rotor (Sorvall, Newton, Conn.). The supernatant is removed, and the mitochondrial pellet is resuspended in 25 ml of the same buffer and centrifuged again. The supernatants are combined and centrifuged for 60 min at 138,000 g in a Sorvall Ultra Pro 80 using a Sorvall T-1270 rotor. The upper lipid layer is removed and the cytosolic supernatant is collected. The microsomal pellet is resuspended in 0.125 M KCl, 0.1 M Tris (pH 7.4) using three homogenization strokes following by a 60-min centrifugation at 138,000 g. The resultant pellet contains liver microsomes. Prior to determination of drug metabolism, the microsomal pellet is resuspended in a suitable incubation buffer (e.g., 0.15 M Tris buffer with 5 mM magnesium chloride, pH 7.4).

For example, the metabolism rate of debrisoquine, a CYP2D6 target drug molecule, can be determined using the following protocol: 0.15-0.30 mg of the microsomal protein is incubated for 60 minutes with 1 mM [guanidine-14C]debrisoquine (0.5 μCi/tube), 1.0 mM NADP, 7.5 mM DL-isocitric acid, 2 U/ml isocitric dehydrogenase, 5 mM MgSO4, and 0.1 M phosphate buffer, pH 7.4, in a final volume of 0.25 ml. The drug metabolism reaction is terminated using 0.02 ml of 70% (v/v) perchloric acid, and the supernatant is analyzed using HPLC analysis. Chromatography is performed with a 150-×4.6-nm column of Supelcosil-5 LC-ABZ protected by a 20-×4-mm column of Supelcosil-5 LC-ABZ and a mobile phase consisting of 12% (v/v) acetonitrile and 88% (v/v) 20 mM sodium perchlorate, pH 2.5, at a flow rate of 2 ml/min. Quantitation of the HPLC eluent is performed using liquid scintillation counting (Anthony B. R., et al., 2000; Drug Metabolism and Disposition 28: 1202-1209).

It will be appreciated that in order to compare the metabolism rate of a drug by two variants of the CYP2D6, the liver microsomes used should be derived from two different individuals, of which one is homozygous for the wild-type form of CYP2D6 and the other is homozygous for the poor metabolizer form of CYP2D6 (e.g., CYP2D6*4).

Thus, the metabolism rate of target drug molecules can be compared (using any of the methods described hereinabove) between the wild-type CYP2D6 and the poor metabolizer variant (e.g., CYP2D6*4). Drug molecules which exhibit reduced metabolism rate using the poor metabolizing variant of CYP2D6 but not using the CYP2D6 wild-type form are identified as capable of inducing and/or accelerating fast progression of liver fibrosis. Once these drugs are recognized as such they should not be prescribed to any individual who is at risk of developing liver fibrosis.

While further reducing the present invention to practice, the present inventor has uncovered that SNPs in additional loci are also associated with fast progression of liver fibrosis. As is further shown in Tables 9, 10 and 11 and described in Example 2 of the Examples section which follows, the adenosine nucleotide-containing allele at nucleotide coordinate 174 of SEQ ID NO:18 (CYP3A5*1 allele), the thymidine nucleotide-containing allele at nucleotide coordinate 1772 of SEQ ID NO:17 (CYP2E1 T-RsaI allele) and/or the cytosine nucleotide-containing allele at nucleotide coordinate 55 of SEQ ID NO:19 (APO E4 allele) are most frequent in the fast fibroser group than in the slow fibroser group of chronic hCV patients.

Thus, according to an additional aspect of the present invention there is provided a method of determining if an individual is predisposed to fast progression of liver fibrosis. The method is effected by determining a presence or absence, in a homozygous or heterozygous form, of at least one fast progression liver fibrosis-associated genotype in a locus selected from the group consisting of CYP3A5, CYP2E1 and APO E or in neighboring loci of the individual, the neighboring loci being in linkage disequilibrium with the locus, thereby determining if the individual is predisposed to fast progression of liver fibrosis.

As used herein the phrase “CYP3A5 locus” refers to a specific DNA sequence region in the human genome encompassing a gene coding for the cytochrome P450 type 3A5 (family 3, subfamily A) and located on chromosome 7 (7q21.1). The genomic sequence of CYP3A5 is included in the nucleic acid sequence set forth by nucleotide coordinates 253080-284889 of GenBank Accession No. NG_{—}000004 as well as GenBank Accession No. AF355800 (SEQ ID NO:18). The CYP3A5 mRNA sequence is set forth by GenBank Accession No. NM_{—}000777.2, and the amino acid sequence of the CYP3A5 polypeptide is set forth by GenBank accession No. NP_{—}000768.

Preferably, the at least one fast progression liver fibrosis-associated genotype in the CYP3A5 locus is the CYP3A5*1 variant, i.e., the adenosine nucleotide-containing allele at nucleotide coordinate 174 as set forth in SEQ ID NO:18 (GenBank Accession No. AF355800).

The phrase “neighboring loci” when used according to this aspect of the present invention with respect to the CYP3A5 locus, refers to describe DNA sequences (either genes or intergenic sequences) that are in close vicinity to the CYP3A5 locus and that include other SNPs that are in linkage disequilibrium with the CYP3A5*1/*3 SNP of the CYP3A5 locus. It will be appreciated that SNPs which are present in neighboring loci but their linkage disequilibrium status with the CYP3A5*1 polymorphism is yet unknown, can be used also along with the present invention. Such SNPs can be found in the genomic sequence set forth in GenBank Accession No. NG_{—}000004, preferably, between nucleotide coordinates 253080-284889 of NG_{—}000004, and/or in the nucleic acid sequence set forth by SEQ ID NO:18.

As used herein the phrase “CYP2E1 locus” refers to a specific DNA sequence region in the human genome encompassing a gene coding for the cytochrome P450, family 2, subfamily E, polypeptide 1 and located on chromosome 10 (10q24.3-qter). The genomic sequence of CYP2E1 is included in the nucleic acid sequence set forth by nucleotide coordinates 135229746-135241501 of GenBank Accession No. NC_{—}000010 as well as in GenBank Accession No. J02843 (SEQ ID NO:17). The CYP2E1 mRNA sequence is set forth by GenBank Accession No. NM_{—}000773, and the amino acid sequence of the CYP2E1 polypeptide is set forth by GenBank accession No. NP_{—}000764.

Preferably, the at least one fast progression liver fibrosis-associated genotype in the CYP2E1 locus is the Thymidine nucleotide-containing allele at nucleotide coordinate 1772 as set forth in SEQ ID NO:17.

The phrase “neighboring loci” when used according to this aspect of the present invention with respect to the CYP2E1 locus, refers to describe DNA sequences (either genes or intergenic sequences) that are in close vicinity to the CYP2E1 locus and that include other SNPs that are in linkage disequilibrium with the CYP2E1 T/C SNP (at nucleotide 1772 as set forth in SEQ ID NO:17) of the CYP2E1 locus. It will be appreciated that SNPs which are present in neighboring loci but their linkage disequilibrium status with the CYP2E1/T allele (at nucleotide 1772 as set forth in SEQ ID NO:17) is yet unknown, can be also used along with the present invention. Such SNPs can be found in the genomic sequence set forth in GenBank Accession No. NC_{—}000010, preferably between nucleotide coordinates 135229746-135241501 of NC_{—}000010, and/or in the nucleic acid sequence set forth by SEQ ID NO:17.

As used herein the phrase APO E locus refers to a specific DNA sequence region in the human genome encompassing a gene coding for the apolipoprotein E which is located on chromosome 19 (19q13.2). The genomic sequence of APO E is included in the nucleic acid sequence set forth by nucleotide coordinates 50100902-50104489 of GenBank Accession No. NC_{—}000019. The APO E mRNA sequence is set forth by GenBank Accession No. NM_{—}000041, and the amino acid sequence of the APO E polypeptide is set forth by GenBank accession No. NP_{—}000032.

Preferably, the at least one fast progression liver fibrosis-associated genotype in the APO E locus is the Cytosine nucleotide-containing allele at nucleotide coordinate 55 as set forth in SEQ ID NO:19.

The phrase “neighboring loci” when used according to this aspect of the present invention with respect to the APO E locus, refers to describe DNA sequences (either genes or intergenic sequences) that are in close vicinity to the APO E locus and that include other SNPs that are in linkage disequilibrium with the APO E E4/E3 SNP (C/T SNP at nucleotide 55 as set forth in SEQ ID NO:19) of the APO E locus. It will be appreciated that SNPs which are present in neighboring loci but their linkage disequilibrium status with the APO E4 allele (C allele at nucleotide 55 of SEQ ID NO:19) is yet unknown, can be used also along with the present invention. Such SNPs can be found in the genomic sequence set forth in GenBank Accession No. NC_{—}000019, preferably, between nucleotide coordinates 50100902 and 50104489 of NC_{—}000019.

The abovementioned genotypes, e.g., the CYP3A5*1 allele (A at nucleotide coordinate 174 of SEQ ID NO:18), the CYP2E1 T allele (T at nucleotide 1772 of SEQ ID NO:17), the APO E4 allele (C at nucleotide coordinate 55 of SEQ ID NO:19) and/or genotypes of SNPs which are in linkage disequilibrium with such SNPs can be detected by any of the SNP detection methods described hereinabove and thus can be used to determine predisposition of individuals to fast progression of liver fibrosis.

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions; illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., Ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (Eds.) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., Ed. (1994); “Culture of Animal Cells—A Manual of Basic Technique” by Freshney, Wiley-Liss, N.Y. (1994), Third Edition; “Current Protocols in Immunology” Volumes I-III Coligan J. E., Ed. (1994); Stites et al. (Eds.), “Basic and Clinical Immunology” (8th Edition), Appleton & Lange, Norwalk, Conn. (1994); Mishell and Shiigi (Eds.), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., Ed. (1984); “Nucleic Acid Hybridization” Hames, B. D., and Higgins S. J., Eds. (1985); “Transcription and Translation” Hames, B. D., and Higgins S. J., Eds. (1984); “Animal Cell Culture” Freshney, R. I., Ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); “Approaches to Gene Mapping in Complex Human Diseases” Jonathan L. Haines and Margaret A. Pericak-Vance eds., Wiley-Liss (1998); “Genetic Dissection of Complex Traits” D.C. Rao and Michael A. Province eds., Academic Press (1999); “Introduction to Quantitative Genetics” D. S. Falconer and Trudy F. C. Mackay, Addison Wesley Longman Limited (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### The CYP2D6*4 Poor Metabolizer is Associated with Fast Progression of Liver Fibrosis in HCV Patients

End-stage liver disease affects 15-20% of the individuals carrying the hepatitis C virus. The mechanisms leading to advanced fibrosis progression rate and to end-stage liver cirrhosis are not yet defined. To test whether the poor metabolizer allele of CYP2D6, CYP2D6*4, can predict fibrosis progression rate, the present inventors have compared the allele frequency of CYP2D6*4 between “slow” and “fast” fibrosers, as follows.

Materials and Methods—

Study cases—Chronic hepatitis C virus (HCV) patients were recruited from the outpatients' clinic of the liver unit at the Department of Gastroenterology (Tel Aviv Sourasky Medical Center, Israel) between August 2003 to January 2004. Inclusion criteria were being of a Caucasian origin and testing positive for HCV RNA using PCR. The patients were interviewed for demographic details (sex, date of birth, age) and clinical data including: age of exposure to the virus, mode of infection, alcohol consumption, genotype of the virus, previous therapy and liver transplantation. The clinical records, laboratory, imaging studies and liver histopathology were reviewed. Exclusion criteria were the presence of a liver disease in addition to HCV, such as autoimmune hepatitis, alcoholic liver disease, positive serology for hepatitis B or HIV. Patients who consumed above 30 gr alcohol per day were also excluded. Blood samples of 19 healthy Caucasian neonates served as controls. The study was approved by the local ethics committee and by the genetic national committee affiliated to the health ministry.

Determination of cirrhosis—The presence of cirrhosis was based on histopathology assessment of liver biopsy or clinical diagnosis in non-biopsed patients.

Histopathology of liver biopsy—Liver biopsies from 36 patients were examined by the same histopathologist. The grade and stage were assessed according to the Batts and Ludwig system (B&L) and were classified as: 1—Portal fibrosis; 2—periportal fibrosis; 3—septal fibrosis; 4—cirrhosis.

Clinical diagnosis of cirrhosis in non-biopsed patients—Clinical diagnosis of cirrhosis was based on signs of portal hypertension as well as laboratory and appropriate radiologic findings.

Definition of “fast” and “slow” fibrosers—The “fast fibrosers” versus “slow fibrosers” were defined according to the Poynard's fibrosis progression model (Poynard, T., et al., 2001. Rates and risk factors of liver fibrosis progression in patients with chronic hepatitis C. J. Hepatol. 34: 730-9). In each case, the duration of the infection period was estimated from the date of exposure until the first liver biopsy. Patients whose disease progressed as predicted by the model curves were classified as “slow fibrosers”. In contrast, patients whose disease progressed faster than expected were classified as “fast fibrosers”. In cases where no liver biopsy was performed, the duration of infection was determined as the period between the date of exposure and the date in which clinical diagnosis was made. In cases where no biopsy was made and there was no evidence of portal hypertension, but the patients were infected long enough to reach cirrhosis according the model, the patients were included and classified as “slow fibrosers”. Cirrhotic patients younger than 45 years of age with unknown date of exposure were considered as “fast fibrosers”, since even if they were infected at birth, they were not expected to reach fibrosis, according to the model, prior to the age of 45.

CYP2D6 assay—Genomic DNA was extracted from peripheral blood by a salting-out procedure (Miller S. A., et al., 1988; Nucleic Acid Res. 16:1215). The presence of the Cytochrome P4502D6*4 mutation (G→A substitution at position 3465 as set forth in SEQ ID NO:6, GenBank Accession No. M33388) was detected using the CYP2D6*4 Forward (SEQ ID NO:2) and CYP2D6*4 Reverse (SEQ ID NO:3) PCR primers and the LightCycler™ Anchor (SEQ ID NO:7) and Mutation (SEQ ID NO:8) probes (see Table 1, hereinbelow), essentially as described elsewhere (Bjerke, J., et al., 2001. Genotyping of Cytochrome P450 2D6*4 Mutation with Fluorescent Hybridization Probes Using LightCycler. “Rapid Cycle Real-Time PCR”. Methods and Applications: Springer Verlog).

TABLE 1
PCR and LightCycler ™ primers

| Primer Name | | Primer |
|---|---|---|
| (SEQ ID NO) | Primer sequence 5′→3′ | modification |
| CYP2D6*4 | CCAACCACTCCGGTGGG | None |
| Forward | | |
| (SEQ ID NO: 2) | | |
| CYP2D6*4 | AATCCTGCTCTTCCGAGGC | None |
| Reverse | | |
| (SEQ ID NO: 3) | | |
| Anchor probe | GTCCAAGAGACCGTTGGGGCGA | 3′-FITC |
| (SEQ ID NO: 7) | | |
| Mutation probe | AGGGGCGTCCTGGGG | 5′-LCRed640; |
| (SEQ ID NO: 8) | | 3′-phoaphate |

Statistical analysis—The SPSS version 11 statistical application was employed for data management and statistical analyses. The rates and proportion for categorical data of the “fast” and “slow” fibroser groups were compared using the χ² test; the means and standard errors for continuous data were compared using the t-test; and the odds ratio were estimated using logistic regression models with 95 percent confidence intervals. Backward logistic regression was employed to examine the relationship of the age, gender, age at exposure, duration of disease and carrier status of CYP2D6*4 with the likelihood of fast fibrosis.

### Experimental Results

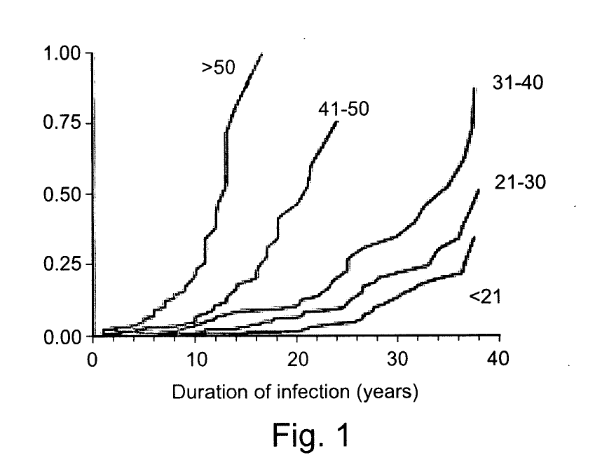

Progression of fibrosis depends on the age and duration of HCV infection—To determine the effect of the age of infection on fibrosis progression the probability of fibrosis progression (as determined using the histopathology) was plotted against the age of infection using data of 2313 HCV patients. As is shown in FIG. 1, patients which were infected with HCV at an age younger than 20 years, did not develop cirrhosis before 40 years of infection. On the other hand, patients which were infected with HCV in the third or fourth decade of their lives, progressed to cirrhosis following 35 years of infection (the rate of fibrosis increased sharply after 30 years of infection in this group). In addition, patients which were infected with HCV on the fifth decade of their lives developed cirrhosis following 20 years of infection. In the latter group, the rate of fibrosis became very steep after 10 years of infection. Lastly, patients which were infected with HCV at an age older than years 50, had the most detrimental course and exhibited cirrhosis following 15 years of infection. Thus, these results demonstrate that the progression of fibrosis is dependent on both the age and the duration since infection.

HCV patients were classified as “fast” and “slow” fibrosers—Fifty HCV Caucasian patients were included in the study. A summary of their demographic data is listed in Table 2, hereinbelow. Of them, 33 patients were classified as “fast fibrosers” and 17 as “slow fibrosers”. Seven patients in the “fast fibrosers” did not undergo a liver biopsy but were diagnosed as having cirrhosis based on clinical diagnosis. Six patients in the “slow group” did not have liver biopsy. One of them, had a diagnosis of cirrhosis based on clinical and imaging evidence. The other five patients had no evidence of cirrhosis (clinical, imaging or laboratory). Since those patients were expected to reach cirrhosis by the time of the evaluation according to the model, they were classified as “slow fibrosers”. Six cirrhotic patients which were younger than 45 years of age and were unable to recall the exposure event and thus, duration of exposure, were classified as “fast fibrosers”. Altogether, the patients in the “fast fibrosers” group were younger and had shorter duration of infection. However, the age of exposure was not significantly different between the two groups.

TABLE 2
Demographic characteristics of the “fast” and
“slow” fibroser groups

| | Fast fibrosers | Slow fibrosers | |
|---|---|---|---|
| | (33) | (17) | P value |
| Male (%) | 13 (39.3%) | 6 (35.2%) | 0.39 |
| Mean age ± SD | 43 ± 7.7 | 63 ± 16.6 | 0.001 |
| Mean age of | 23.7 ± 10.3* | 27.1 ± 12.6 | 0.34 |
| exposure ± SD | | | |
| Mean duration of | 18.7 ± 8.2* | 36.4 ± 12.4 | <0.0001 |
| infection | | | |
| years ± SD | | | |
| Liver trans- | 7 (21%) | 0 | <0.0001 |
| plantation (%) | | | |
| Mean stage of | 3.9 ± 0.27** | 2.4 ± 3.6*** | 0.017 |
| fibrosis by | | | |
| biopsy ± SD | | | |

Demographic characteristics are provided for 17 “slow” fibrosers and 33 “fast” fibrosers unless otherwise noted.
*= 27 patients;
**= 26 patients;
***= 10 patients.

The CYP2D6*4 allele of the Cytochrome P450-2D6 is associated with cirrhosis progression—In order to determine whether the P450-2D6*4 mutation is associated with fast progression of liver cirrhosis, the presence of the CYP2D6*4 allele was determined in DNA obtained from “fast” and “slow” fibrosers, as well as in DNA obtained from healthy neonatal controls. As is shown in Table 3, hereinbelow, the prevalence of the CYP2D6*4 allele in the control group was 10.5%, with no significant statistical difference from its prevalence in the “slow” group (14.7%). In contrast, the prevalence of the CYP2D6*4 allele in the “fast” group was significantly higher 37.8% (P-value=0.0166).

TABLE 3
The prevalence of homozygotes, heterozygotes and carriers
in the “fast” and “slow fibrosers”

| CYP2D6*4 carrier | | | |
|---|---|---|---|
| status | Fast fibrosers | Slow fibrosers | Healthy controls |
| Allele (%) | 25/66 (37.8%) | 5/34 (14.7%) | 4/38 (10.5%) |
| Homozygote (%) | 6/33 (18.1%) | 1/17 (5.8%) | 0/19 (0%) |
| Heterozygote (%) | 13/33 (39.3%) | 3/17 (17.6%) | 4/19 (21%) |
| Homozygote or | 19/33 (57.6%) | 4/17 (23.6%)* | 4/19 (21%) |
| heterozygote (%) | | | |
| (carrier group) | | | |
| None (%) | 14/33 (42.4%) | 13/17 (76.4%) | 15/19 (78.9%) |

Table 3: Prevalence of CYP2D6*4 in “fast” and “slow” fibroser groups. Homozygotes = refers to the CYP2D6*4 allele; none = homozygotes to the wildtype allele; carrier group = refers to individuals who carry at least one allele of the CYP2D6*4.
*= P value = 0.022

In addition, logistic regression analysis revealed that the frequency of the CYP2D6*4 carriers (i.e., heterozygous or homozygous individuals) was significantly higher in the “fast” fibroser group (57.6%) than in the “slow” fibroser group (23.6%, P value=0.022, Table 3, hereinabove). In addition, the odd ratio of the CYP2D6*4 carrier state was 11.7 (C.I. 95%, confidence interval 1.4-95.27, Table 4, hereinbelow).

In contrast, the duration of infection was inversely related to fast fibrosis (Table 4, hereinbelow). On the other hand, younger age of exposure, gender and age were not significantly associated with accelerated rate of fibrosis (not shown).

TABLE 4
The association between CYP2D6*4 carrier state,
duration of infection, and rate of fibrosis

| Independent variant | OR | 95% CI | Significance |
|---|---|---|---|
| CYP2D6*4 carrier state | 11.7 | 1.4-95.27 | 0.021 |
| Duration of infection | 0.84 | 0.75-0.93 | 0.002 |

CI = confidence interval; OR = odd ratio.

Thus, these results demonstrate that HCV patients which carry the CYP2D6*4 mutation have increased risk to progress fast towards liver cirrhosis.

Thus, the present inventors have uncovered, for the first time, that genotypes in the CYP2D6 gene are significant predictors of liver fibrosis progression rate in HCV patients.

Analysis and discussion—Significant advances in the treatment of HCV infection have occurred over the past decade and the pool of treatable patients has been expanded. Patients with mild disease (asymptomatic, minimal fibrosis on biopsy, infected at young age) were traditionally considered to run an indolent course. Subsequently, in the light of the severe side effects of therapy, quality of life matters and cost effectiveness issues, eligibility of these patients for treatment remains controversial (Heathcote J. Antiviral therapy for patients with chronic hepatitis C. Semin. Liver Dis. 2000, 20: 185-99). Knowing that some of the patients have detrimental courses with rapid progression to cirrhosis [Poynard, 2001 (Supra)] it is of great importance to identify these patients and initiate treatment early in the course of the infection.

The results of the present invention provide evidence that CYP2D6*4, the poor metabolizer genotype, is significantly associated with accelerated rate of fibrosis. The prevalence of the allele was significantly higher in the “fast fibrosers” than it was in the “slow fibrosers”. However there was no significant difference between the prevalence of the allele in the slow group and the controls. Moreover, the logistic regression analysis demonstrated that the carrier state of the CYP2D6*4 allele, possesses a higher risk for rapid progression to cirrhosis.

The significant differences between the “fast” and “slow” groups regarding the duration of infection and stage of fibrosis (Table 2, hereinabove), validated the methodology of “fast” and “slow” classification. In this respect, it was previously demonstrated that the median estimated duration of infection for progression to cirrhosis is 30 years [Poynard, 1997 (Supra)]. In the present study, the median period of infection in the “slow” group was 36.4 years as compared with 18.7 years of infection in the “fast” group.

The precise role of CYP2D6 in the fibrogenetic process in hepatitis C is yet to be clarified. Lately the role CYP450 superfamily and CYP2 family in arachidonic acid and eicosanoids metabolism has been increasingly recognized. The exact implication of each enzyme of the complex in the various metabolic pathways of this substances are still under extensive investigation (Nebert, D. W, et al., 2002. Clinical importance of the cytochromes P450. Lancet. 360(9340): 1155-62). It was previously demonstrated that arachidonic acid upregulated collagen type 1 synthesis via transcriptional activation of the collagen gene in hepatic stellate cell line (Nieto N, et al., 2000. Ethanol and arachidonic acid increase alpha 2(I) collagen expression in rat hepatic stellate cells overexpressing cytochrome P450 2E1. Role of H₂O₂ and cyclooxygenase-2. J. Biol. Chem. 275: 20136-45). Therefore lack of CYP2D6 activity might reduce arachidonic acid degradation and thus increase collagen type 1 production, from hepatic stellate cells.

### Example 2

### Involvement of SNP in CYP2D6, CYP3A5, CYP2E1 and APO E in Predisposition to Fast Fibrosis

To identify additional risk factors for fast progression of liver fibrosis and cirrhosis additional 32 Caucasians patients of Jewish origin with chronic Hepatitis C Virus (HCV) infection were recruited for the study. The 32 patients were classified as “fast fibrosers” (14 patients) or “slow fibrosers” (18 patients) according to the study protocol described under Material and Methods of Example 1, hereinabove. Altogether, at present, 82 patients with chronic HCV are included in the study.

Material and Methods

Study subjects and blood samples—The 32 chronic hCV patients were interviewed regarding demographic and clinical data, and their medical records were reviewed as described under Material and Methods in Example 1, hereinabove. Blood samples were withdrawn from each patient and DNA was extracted from peripheral blood lymphocytes as described under Material and Methods in Example 1, hereinabove.

Non-Alcoholic Fatty Liver Disease (NAFLD) patients—To date, 15 Caucasian NAFLD patients of Jewish origin were enrolled. The patients were interviewed (similarly to the hCV patients) and all medical records were reviewed and documented. Blood samples were withdrawn and DNA was extracted.

Molecular analysis for the presence of SNPs in the CYP2D6, CYP3A5, CYP2E1, and APO E genes in chronic hCV patients—Table 5, hereinbelow, presents the PCR primers used to amplify the relevant PCR products including the polymorphic nucleotides.

Cytochrome P4502D6*4 mutation (G→A substitution at position 3465 as set forth in SEQ ID NO:6, GenBank Accession No. M33388)—was detected as described in Example 1, hereinabove. Heterozygotes for CYP2D6 are *4 (A allele)/WT (G allele) and Homozygotes are *4 (A allele)/*4 (A allele).

CYP2E1 SNP G→C in the CYP2E1 Promoter at nucleotide 1532 as set forth in SEQ ID NO:17, GenBank Accession No. J02843)—was detected by amplifying a genomic DNA with the forward (SEQ ID NO:11) and reverse (SEQ ID NO:12) PCR primers listed in Table 5, hereinbelow. Following PCR amplification, the G→C polymorphism (GTGCAG→CCTGCAG; underlined nucleotides are polymorphic) was detected by digesting the PCR product (413 bp) with the PstI restriction enzyme which recognizes the CTGCAG sequence (the underlined C is the polymorphic nucleotide). Thus, in the absence of SNP (wildtype, common allele of G at position 1532 of SEQ ID NO:17), PstI digestion results in a single fragment of 413 bp. On the other hand, in the presence of SNP (rare allele, C at position 1532 of SEQ ID NO:17), PstI digestion results in two fragments of 118 and 295 bp.

CYP2E1 SNP C→T at the CYP2E1 Promoter at nucleotide 1772 as set forth in SEQ ID NO:17, GenBank Accession No. J0284)—was detected by amplifying a genomic DNA with the forward (SEQ ID NO:11) and reverse (SEQ ID NO: 12) PCR primers listed in Table 5, hereinbelow. Following PCR amplification, the C→T polymorphism which changes a restriction site to RsaI GTAC→GTAT (underlined C and T are the polymorphic nucleotides) is detected by digesting the PCR product (413 bp) with the RsaI restriction enzyme. Thus, in the absence of SNP (wildtype, common allele of C at position 1772 of SEQ ID NO:17), RsaI digestion results in two fragments of 61 and 352 bp. On the other hand, in the presence of SNP (rare allele of T at position 1772 of SEQ ID NO:17), RsaI digestion results in a single fragment of 413 bp.

CYP3A5*3 (Intron 3) SNP A→G at position 174 of SEQ ID NO:18, GenBank Accession No. AF355800—was detected using the forward (SEQ ID NO:13) and reverse (SEQ ID NO:14) PCR primers listed in Table 5, hereinbelow. Following PCR amplification, the A→G polymorphism was detected by digesting the PCR product (200 bp) with the DdeI restriction enzyme, which recognizes the CTNAG sequence (N=any nucleotide). Thus, in the absence of SNP (CYP3A5*1 allele with nucleotide A at position 174 of SEQ ID NO:18), DdeI digestion results in two fragments of 129 and 71 bp. On the other hand, in the presence of SNP (CYP3A5*3 allele, G nucleotide at position 174 of SEQ ID NO:18), DdeI digestion results in three fragments of 22, 71 and 107 bp. Variant G named CYP3A5*3 and variant A named CYP3A5*1. Heterozygotes for CYP3A5 are *3/*1 (G/A at position 174 of SEQ ID NO:18), homozygotes are *3/*3 (GIG at position 174 of SEQ ID NO:18), none (homozygotes of *1 allele) exhibit A/A at position 174 of SEQ ID NO:18). For additional information see Shuichi Fukuen et al., 2002, Novel detection assay by PCR-RFLP and frequency of the CYP3A5 SNPs, CYP3A5*3 and *6, in a Japanese population, Pharmacogenetics, 12: 331-334; which is fully incorporated herein by reference.

APO E4 variant SNP T→C at position 55 of SEQ ID NO:19 (which corresponds to nucleotide 2880 at GenBank Accession No. NC_{—}000019:50100902-50104489)—was detected using the forward (SEQ ID NO:15) and reverse (SEQ ID NO:16) PCR primers listed in Table 5, hereinbelow. Following PCR amplification, the T→C polymorphism was detected by digesting the PCR product (227 bp; SEQ ID NO:19) with the Hin6I restriction enzyme, which recognizes the GCGC sequence. Thus, in the absence of SNP (wildtype, common allele of T at position 55 of SEQ ID NO:19), Hin6I digestion results in the following fragments: 21, 16, 91, 18 and 81 bp. On the other hand, in the presence of APO E4 variant (rare allele of C at position 55 of SEQ ID NO:19), Hin6I digestion results in the following fragments: 21, 16, 19, 72, 18 and 81 bp (thus the 91 bp fragment is cut into two fragments of 19 and 72 bp). This SNP changes amino acid residue Cys (codon TGC) to Arg (codon CGC) at position 130 of the Apo E protein (GenBank Accession No. NP_{—}000032).

APO E2 variant SNP C→T at position 193 of SEQ ID NO:19 (which corresponds to nucleotide 3018 at GenBank Accession No. NC_{—}000019:50100902-50104489; SNP rs7412 at the NCBI SNP database)—was detected using the forward (SEQ ID NO:15) and reverse (SEQ ID NO:16) PCR primers listed in Table 5, hereinbelow. Following PCR amplification, the C→T polymorphism was detected by digesting the PCR product (227 bp; SEQ ID NO:19) with the Hin6I restriction enzyme, which recognizes the GCGC sequence. Thus, in the absence of SNP (wildtype, common allele of C at position 193 of SEQ ID NO:19), Hin6I digestion results in the following fragments: 21, 16, 91, 18, 48 and 33 bp. On the other hand, in the presence of APO E2 variant (rare allele of T at position 193 of SEQ ID NO:19), Hin6I digestion results in the following fragments: 21, 16, 91, 18 and 81 (thus, the disappearance of the Hin6I restriction site generated an 81 bp fragment instead of the 48 and 33 bp fragments). This SNP changes amino acid residue Arg (codon CGC) to Cys (codon TGC) at position 176 of the Apo E protein (GenBank Accession No. NP_{—}000032).

TABLE 5
PCR primers

| Primer Name | | |
|---|---|---|
| (SEQ ID NO) | Primer sequence 5′→3′ | SNP |
| CYP2E1 | CCAGTCGAGTCTACATTGTCA | G→C (GTGCAG) at |
| (Promoter) Forward | | nucleotide coordinate |
| (SEQ ID NO:11) | | 1532 of SEQ ID NO:17 |
| CYP2E1 | CCAGTTAGAAGACAGAATGAA | |
| (Promoter) Reverse | | |
| (SEQ ID NO:12) | | |
| CYP2E1 | CCAGTCGAGTCTACATTGTCA | C→T (GTAC) at |
| (Promoter) Forward | | nucleotide coordinate |
| (SEQ ID NO:11) | | 1772 of SEQ ID NO:17 |
| CYP2E1 | CCAGTTAGAAGACAGAATGAA | |
| (Promoter) Reverse | | |
| (SEQ ID NO:12) | | |
| CYP3A5 (Intron 3) | CTTTAAAGAGCTCTTTTGTCTCTC | CYP3A5*3 |
| Forward primer | | A→G at nucleotide |
| (6956Fm; SEQ ID NO:13) | | coordinate 174 of |
| CYP3A5 (Intron 3) | CCAGGAAGCCAGCTTTGAT | SEQ ID NO:18 |
| Reverse primer | | |
| (7155R; SEQ ID NO:14) | | |
| APO E | TCCAAGGAGCTGCAGGCGGCGCA | APO E4 T→C SNP at |
| Forward primer | | nucleotide 55 of |
| (SEQ ID NO:15) | | SEQ ID NO:19 |
| APO E | ACAGAATTCGCCCCGGCCTGGTACACTGCCA | |
| Reverse primer | | |
| (SEQ ID NO:16) | | |
| APO E | TCCAAGGAGCTGCAGGCGGCGCA | APO E2 C→T SNP at |
| Forward primer | | nucleotide 193 of |
| (SEQ ID NO:15) | | SEQ ID NO:19 |
| APO E | ACAGAATTCGCCCCGGCCTGGTACACTGCCA | |
| Reverse primer | | |
| (SEQ ID NO:16) | | |

Table 5:
PCR primers used for genotype the CYP2E1, CYP3A5, and Apo E polymorphisms. The underlined C in SEQ ID NO:13 was created to form a restriction site for the DdeI restriction enzyme. The italic sequence in SEQ ID NO:16 (ACAGAATTC) is a tail added to the primer, such that the primer gene specific sequence is GCCCCGGCCTGGTACACTGCCA which corresponds to nucleotides 3043-3022 on GenBank Accession No. NC_000019: 50100902-50104489; the forward primer SEQ ID NO:15 corresponds to nucleotides 2826-2848 on GenBank Accession No. NC_000019: 50100902-50104489..

Experimental Results

Demographic characteristics of the additional 32 hCV patients—Table 6, hereinbelow, summarizes the demographic data of the additional 30 hCV patients recruited for the present study. As is shown in the Table, the male gender was associated with fast fibrosis. In addition, while the average duration of infection in the slow fibrosers group was 23.7±10.36, the duration of infection in the fast fibrosers group was 15.43±4.6.

TABLE 6
Demographic characteristics of study cases

| Slow Fibrosers | | | | Fast Fibrosers | | | |
|---|---|---|---|---|---|---|---|
| | | | Age at the | | | | Age at the |
| | | Duration of | time of | | | Duration of | time of |
| | Age | infection | infection | | Age | infection | infection |
| Sex | (years) | (years) | (years) | Sex | (years) | (years) | (years) |
| F | 39 | 13 | 26 | M | 35 | 20 | 15 |
| M | 80 | 30 | 50 | M | 32 | 15 | 17 |
| M | 52 | 30 | 22 | M | 52 | 18 | 34 |
| F | 59 | 38 | 21 | M | 29 | 14 | 15 |
| F | 46 | 40 | 6 | M | 29 | 13 | 16 |
| F | 38 | 31 | 7 | F | 33 | 23 | 10 |
| F | 57 | 37 | 20 | M | 38 | 23 | 15 |
| M | 37 | 13 | 24 | F | 49 | 7 | 42 |
| M | 27 | 15 | 12 | M | 38 | 18 | 20 |
| F | 49 | 15 | 34 | F | 66 | 15 | 51 |
| M | 48 | 11 | 37 | M | 37 | 10 | 27 |
| M | 42 | 27 | 17 | M | 45 | 13 | 32 |
| M | 45 | 21 | 24 | M | 33 | 16 | 17 |
| F | 56 | 39 | 17 | F | 52 | 11 | 41 |
| M | 37 | 14 | 23 | | | | |
| M | 45 | 14 | 31 | | | | |
| M | 56 | 22 | 34 | | | | |
| F | 47 | 16 | 31 | | | | |
| Ave. M | Ave. | Ave. | Ave. | Ave. M | Ave. | Ave. | Ave. |
| 55.5% | 47.7 ± | 23.7 ± | 24.2 ± | 71.4% | 40.6 ± | 15.43 ± | 25.14 ± |
| (10/18) | 11.6 | 10.36 | 10.93 | (10/14) | 10.78 | 4.6 | 12.75 |

Table 6: Shown are the demographic characteristics of the additional 32 hCV patients recruited for the present study. Note the high frequency of males among the fast fibroser group and the short duration of infection. None of the fast or slow fibrosers underwent liver transplantation.
Ave = average.

Genotyping of additional 32 hCV patients revealed high frequency of heterozygotes to the CYP2D6*4 variant among fast fibrosers—The poor metabolizer variant of CYP2D6 was found to associated with fast fibrosis also among the additional 32 patients. As is shown in Table 7, hereinbelow,

TABLE 7
The prevalence ofCYP2D6*4 among the “fast” and “slow
fibrosers” of the additional 32 hCV patients

| CYP2D6*4 carrier status | Fast fibrosers | Slow fibrosers |
|---|---|---|
| Allele (%) | 6/28 (21.4%) | 4/36 (11.1%) |
| Homozygote (%) | 1/14 (7.1%) | 0 |
| Heterozygote (%) | 4/14 (28.57%) | 4/18 (22.2%) |
| Homozygote or heterozygote (%) | 5/14 (35.71%) | 4/18 (22.22%) |
| (carrier group) | | |
| None (%) | 9/14 (64.29%) | 14/18 (77.78%) |

Table 7: Prevalence of CYP2D6*4 in “fast” and “slow” fibroser groups among the additional 32 hCV patients recruited for the present study. Homozygotes = refers to the CYP2D6*4 allele; none = homozygotes to the wildtype allele; carrier group = refers to individuals who carry at least one allele of the CYP2D6*4.

TABLE 8
The prevalence ofCYP2D6*4 among the “fast” and “slow
fibrosers” in the overall hCV cases of the present study

| CYP2D6*4 carrier | | | |
|---|---|---|---|
| status | Fast fibrosers | Slow fibrosers | Healthy controls |
| Allele (%) | 31/94 (32.98%) | 9/70 (12.85%) | 4/38 (10.5%) |
| Homozygote (%) | 7/47 (14.8%) | 1/35 (2.85%) | 0/19 (0%) |
| Heterozygote (%) | 17/47 (36.1%) | 7/35 (20%) | 4/19 (21.05%) |
| Homozygote or | 24/47 (51.06%) | 8/35 (22.86%) | 4/19 (21.05%) |
| heterozygote (%) | | | |
| (carrier group) | | | |
| None (%) | 23/47 (48.94%) | 27/35 (77.14%) | 15/19 (78.95%) |

Table 8: Prevalence of CYP2D6*4 in “fast” and “slow” fibroser groups in the overall hCV cases of the present study. Homozygotes = refers to the CYP2D6*4 allele; none = homozygotes to the wildtype allele; carrier group = refers to individuals who carry at least one allele of the CYP2D6*4.

As is shown in Table 8, hereinabove, when the overall genotype data of the hCV cases of the present invention was classified according to fast or slow fibrosers, a significant different was obtained between both the allele frequency and the homozygotes, heterozygotes or overall carriers of the CYP2D6*4 allele. For example, while the prevalence of the CYP2D6*4 allele among the fast fibrosers was ˜33%, the prevalence of that allele among the slow fibrosers or the healthy controls was 13%. In addition, the overall carrier status of the CYP2D6*4 allele was more than two times higher among the fast fibrosers as compared with the slow fibrosers or healthy controls.

These results provide further support for the previous findings presented in Example 1, hereinabove, and demonstrate that hCV patients which carry the CYP2D6*4 mutation have increased risk to progress fast towards liver fibrosis and cirrhosis.

Genotype state of SNPs in the CYP3A5, CYP2E1 and APO E loci among the fast and slow fibrosers—The genotype state of additional SNPs in the CYP3A5, CYP2E1 and APO E loci was determined for the additional 32 hCV patients of the present study as described under the Material and Methods section hereinabove, and is summarized in Tables 9-11 hereinbelow.

TABLE 9
The prevalence of CYP3A5*3 allele among the “fast” and “slow
fibrosers” in the additional 32 hCV cases of the present study

| CYP3A5*3 carrier status | Fast fibrosers | Slow fibrosers |
|---|---|---|
| Allele (CYP3A5*3) | 24/28 (85.7%) | 34/36 (94.4%) |
| Homozygote | 10/14 (71.4%) | 16/18 (88.9%) |
| (CYP3A5*3/*3) | | |
| Heterozygote | 4/14 (28.6%) | 2/18 (11.1%) |
| (CYP3A5*3/*1) | | |
| Homozygote or heterozygote | 14/14 (100%) | 18/18 (100%) |
| (CYP3A5*3 carrier group) | | |
| None (CYP3A5*1/*1) | 0 | 0 |

Table 9: Prevalence of CYP3A5*3 in “fast” and “slow” fibroser groups in the additional 32 hCV cases of the present study. Homozygotes - refers to individuals exhibiting the CYP3A5 *3/*3 genotype; None - refers to individual exhibiting the CYP3A5 *1/*1 genotype; heterozygotes - refers to individuals exhibiting the CYP3A5 *1/*3 genotype; Carrier group = refers to individuals who carry at least one allele of the CYP3A5*3.

As is shown in Table 9, hereinabove, a slight difference in the frequency of the CYP3A5*3 allele was observed between the fast fibrosers and the slow fibrosers, such that the CYP3A5*1 allele is more prevalent among fast fibrosers 14.3%) than among slow fibrosers (5.6%).

TABLE 10
The prevalence of CYP2E1 G→C (PstI) and C→T (RsaI)
SNPs among the “fast” and “slow fibrosers” in
the additional 32 hCV cases of the present study

| | |
|---|---|
| Fast fibrosers | Slow fibrosers |

CYP2E1 G→C (PstI) carrier status

| | | |
|---|---|---|
| Allele (C; PstI) | 1/28 (3.57%) | 1/36 (2.78%) |
| Homozygote (C/C; PstI) | 0 | 0 |
| Heterozygote (G/C; PstI) | 1/14 (7.14%) | 1/18 (5.55%) |
| Homozygote or heterozygote | 1/14 (7.14%) | 1/18 (5.55%) |
| (carrier group of C; PstI) | | |
| None (WT; G/G; PstI) | 13/14 (92.86%) | 17/18 (94.44%) |

CYP2E1 C→T (RsaI) carrier status

| | | |
|---|---|---|
| Allele (T; RsaI) | 1/28 (3.57%) | 0 |
| Homozygote (T/T; RsaI) | 0 | 0 |
| Heterozygote (C/T; RsaI) | 1/14 (7.14%) | 0 |
| Homozygote or heterozygote | 1/14 (7.14%) | 0 |
| (carrier group of T; RsaI) | | |
| None (WT; C/C; RsaI) | 13/14 (92.86%) | 18/18 (100%) |

Table 10: The prevalence of CYP2E1 G→C (PstI; nucleotide 1532 as set forth in SEQ ID NO: 17) and C→T (RsaI; nucleotide 1772 as set forth in SEQ ID NO: 17) SNPs among the “fast” and “slow fibrosers” in the additional 32 hCV cases of the present study.

As is shown in Table 10, hereinabove, no significant difference was observed in the frequency of the CYP2E1 G→C (PstI; nucleotide 1532 as set forth in SEQ ID NO:17) SNP among the fast and slow fibrosers. On the other hand, there was a difference in the frequency of the CYP2E1 C→T (RsaI; nucleotide 1772 as set forth in SEQ ID NO:17) between the fast and slow fibrosers. Thus, while in the slow fibrosers all patients were homozygotes to the wild type allele (C at nucleotide 1772 of SEQ ID NO:17) the frequency of heterozygotes to the T allele (at nucleotide 1772 of SEQ ID NO:17) was ˜7% among the fast fibrosers.

TABLE 11
The prevalence of APO E4 and APO E2 alleles among
the “fast” and “slow flbrosers” in
the additional 32 hCV cases of the present study

| | |
|---|---|
| Fast fibrosers | Slow fibrosers |

APO E4 T→C (at nucleotide 55 of SEQ ID NO: 19) carrier status

| | | |
|---|---|---|
| Allele (C) | 2/28 (7.14%) | 1/36 (2.78%) |
| Homozygote (C/C; variant | 0 | 0 |
| E4/E4) | | |
| Heterozygote (T/C; variant | 2/14 (14.29%) | 1/18 (5.56%) |
| E4/E3) | | |
| Homozygote or heterozygote | 2/14 (14.29%) | 1/18 (5.56%) |
| (carrier group of C) | | |
| None (WT; T/T; variant | 12/14 (85.71%) | 17/18 (94.44%) |
| E3/E3) | | |

APO E2 C→T (at nucleotide 193 of SEQ ID NO: 19) carrier status

| | | |
|---|---|---|
| Allele (T) | 2/28 (7.14%) | 3/36 (8.33%) |
| Homozygote (T/T; variant | 0 | 0 |
| E2/E2) | | |
| Heterozygote (C/T; variant | 2/14 (14.29%) | 3/18 (16.67%) |
| E2/E3) | | |
| Homozygote or heterozygote | 2/14 (14.29%) | 3/18 (16.67%) |
| (carrier group of T) | | |
| None (WT; C/C; variant | 12/14 (85.71%) | 15/18 (83.33%) |
| E3/E3) | | |

Table 11: The prevalence of APO E4 (T→C change at nucleotide 55 of SEQ ID NO: 19) and APO E2 (C→T change at nucleotide 193 of SEQ ID NO: 19) SNPs among the “fast” and “slow fibrosers” in the additional 32 hCV cases of the present study.

As is shown in Table 11, hereinabove, while there was no significant difference in the prevalence of the APO E2 allele between the fast (˜7%) and slow (˜8%) fibrosers, there was a difference in the prevalence of the APO E4 allele between the fast (˜7%) and the slow (˜3%) fibrosers. In addition, an almost three times difference was observed in the frequency of the carriers of the APO E4 alleles (heterozygote individuals). While in the fast fibrosers the heterozygote frequency was 14.3%, in the slow fibrosers the frequency of the heterozygotes was 5.5%.

Altogether, these results demonstrate the high association of the CYP2D6*4 Cytochrome P4502D6*4 mutation (G→A substitution at position 3465 as set forth in SEQ ID NO:6) with fast progression of liver fibrosis and suggest the use of such a polymorphism in determining predisposed to fast progression of liver fibrosis.

In addition, the genotype data of the other candidate genes (e.g., CYP3A5, CYP2E1 and APO E) suggest the use of SNPs in these genes and loci for determining the predisposition of an individual to fast progression of liver fibrosis. Thus, the adenosine nucleotide-containing allele at nucleotide coordinate 174 as set forth in SEQ ID NO:18 (CYP3A5*1 allele), the thymidine nucleotide-containing allele at nucleotide coordinate 1772 as set forth in SEQ ID NO:17 (CYP2E1 T-RsaI allele) and/or the cytosine nucleotide-containing allele at nucleotide coordinate 55 as set forth in SEQ ID NO:19 (APO E4 allele) can be used for determining predisposition towards fast progression of liver fibrosis.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### REFERENCES CITED

### Additional References are Cited in the Text

1. Lauer G M, Walker B D. Hepatitis C virus infection. N Eng J Med 2001; 345(1): 41-52.
2. Seeff L B, Miller R N, Rabkin C S, Buskell-Bales Z, Straley-Eason K D, Smoak B L, Johnson L D, Lee S R, Kaplan E L. 45-year follow-up of hepatitis C virus infection in healthy young adults. Ann Intern Med. 2000 Jan. 18; 132(2):105-11.
3. Poynard T, Bedossa P, Opolon P. Natural history of liver fibrosis progression in patients with chronic hepatitis C. The OBSVIRC, METAVIR, CLINIVIR, and DOSVIRC groups. Lancet. 1997 Mar. 22; 349(9055):825-32.
4. Feldman M, Friedman L S, Sleisenger M H. Sleisenger & Fordtran's “Gastrointestinal and Liver disease” 7th Edition. SAUNDERS An Imprint of Elsevier Science. 2002.
5. Bataller R, North K E, Brenner D A. Genetic polymorphisms and the progression of liver fibrosis: a critical appraisal. Hepatology. 2003 March; 37(3):493-503.
6. Smith B C, Gorve J, Guzail M A, Day C P, Daly A K, Burt A D, Bassendine M F. Heterozygosity for hereditary hemochromatosis is associated with more fibrosis in chronic hepatitis C. Hepatology. 1998 June; 27(6):1695-9.
7. Wozniak M A, Itzhaki R F, Faragher E B, James M W, Ryder S D, Irving W L; Trent HCV Study Group. Apolipoprotein E-epsilon 4 protects against severe liver disease caused by hepatitis C virus. Hepatology. 2002 August; 36(2):456-63.
8. Hasler J A. Pharmacogenetics of cytochromes P450. Mol Aspects Med. 1999 February-April; 20(1-2):12-24, 25-137.
9. Lee H C, Lee H S, Jung S H, Yi S Y, Jung H K, Yoon J H, Kim C Y. Association between polymorphisms of ethanol-metabolizing enzymes and susceptibility to alcoholic cirrhosis in a Korean male population. J Korean Med. Sci. 2001 December; 16(6):745-50.
10. Agundez J A, Gallardo L, Ledesma M C, Lozano L, Rodriguez-Lescure A, Pontes J C, Iglesias-Moreno M C, Poch J, Ladero J M, Benitez J. Functionally active duplications of the CYP2D6 gene are more prevalent among larynx and lung cancer patients. Oncology. 2001; 61(1):59-63.
11. Silvestri L, Sonzogni L, De Silvestri A, Gritti C, Foti L, Zavaglia C, Leveri M, Cividini A, Mondelli M U, Civardi E, Silini E M. CYP enzyme polymorphisms and susceptibility to HCV-related chronic liver disease and liver cancer. Int J Cancer. 2003 Apr. 10; 104(3):310-7.
12. Agundez J A, Ledesma M C, Benitez J, Ladero J M, Rodriguez-Lescure A, Diaz-Rubio E, Diaz-Rubio M. CYP2D6 genes and risk of liver cancer. Lancet. 1995 Apr. 1; 345(8953):830-1.
13. Batts K P, Ludwig J. Chronic hepatitis. An update on terminology and reporting. Am J Surg Pathol. 1995 December; 19(12):1409-17.
14. Poynard T, Ratziu V, Charlotte F, Goodman Z, McHutchison J, Albrecht J. Rates and risk factors of liver fibrosis progression in patients with chronic hepatitis c. J Hepatol. 2001 May; 34(5):730-9.
15. Bjerke J, Chang C C, Schur C, Wong S, Nuwayhid N. Genotyping of Cytochrome P450 2D6*4 Mutation with Fluorescent Hybridization Probes Using LightCycler. “Rapid Cycle Real-Time PCR. Methods and Applications: Springer Verlog 2001.
16. Heathcote J. Antiviral therapy for patients with chronic hepatitis C. Semin Liver Dis. 2000; 20(2): 185-99.
17. Nebert D W, Russell D W. Clinical importance of the cytochromes P450. Lancet. 2002 Oct. 12; 360(9340):1155-62.
18. Nieto N, Greenwel P, Friedman S L, Zhang F, Dannenberg A J, Cederbaum A I. Ethanol and arachidonic acid increase alpha 2(I) collagen expression in rat hepatic stellate cells overexpressing cytochrome P450 2E1. Role of H2O2 and cyclooxygenase-2. J Biol. Chem. 2000 Jun. 30; 275(26):20136-45

1-49. (canceled)

50. A method of determining if an individual is predisposed to fast progression of liver fibrosis, the method comprising determining a presence or absence, in a homozygous or heterozygous form, of at least one fast progression liver fibrosis-associated genotype in the CYP2D6 locus or in neighboring loci of the individual, said neighboring loci being in linkage disequilibrium with said CYP2D6 locus, thereby determining if the individual is predisposed to fast progression of liver fibrosis.

51. The method of claim 50, wherein the individual is infected with an hepatitis C virus.

52. The method of claim 50, wherein said at least one fast progression liver fibrosis-associated genotype in the CYP2D6 locus is an adenosine nucleotide-containing allele of the CYP2D6*4 SNP as set forth in SEQ ID NO:1.

53. The method of claim 50, wherein said at least one fast progression liver fibrosis-associated genotype encodes a truncated CYP2D6 polypeptide.

54. The method of claim 51, wherein said presence of said genotype is indicative of increased predisposition risk of developing fast progression of liver fibrosis in the individual.

55. The method of claim 51, wherein said presence of said genotype is indicative of increased predisposition risk of developing liver cirrhosis.

56. A kit for determining if an individual is predisposed to fast progression of liver fibrosis, the kit comprising at least one reagent for determining a presence or absence in a homozygous or heterozygous form, of at least one fast progression liver fibrosis-associated genotype in the CYP2D6 locus or in neighboring loci of the individual, said neighboring loci are in linkage disequilibrium with said CYP2D6 locus.

57. A method of preventing fast progression of liver fibrosis in an individual in need thereof, the method comprising administering to the individual an agent capable of upregulating the expression level and/or activity of CYP2D6 in the liver of the individual, thereby preventing fast progression of liver fibrosis in the individual.

58. The method of claim 57, wherein said individual is infected with an hepatitis C virus.

59. The method of claim 57, wherein said individual is suffering from a disease selected from the group of an hepatitis viral infection, an hepatotoxicity, a liver cancer, a non alcoholic fatty liver disease (NAFLD), an autoimmune disease, a metabolic liver disease, and a disease with secondary involvement of the liver.

60. The method of claim 57, wherein said upregulating is effected by at least one approach selected from the group consisting of:
(a) expressing in liver cells of the individual an exogenous polynucleotide encoding at least a functional portion of CYP2D6;
(b) increasing expression of endogenous CYP2D6 in liver cells of the individual;
(c) increasing endogenous CYP2D6 activity in liver cells of the individual; and
(d) administering CYP2D6-expressing cells into the liver of the individual.

61. The method of claim 60, wherein said CYP2D6 is a polypeptide at least 75% identical to the polypeptide set forth by SEQ ID NO:4 as determined using the BlastP software of the National Center of Biotechnology Information (NCBI) using default parameters.

62. The method of claim 60, wherein said CYP2D6 is set forth by SEQ ID NO:4.

63. The method of claim 60, wherein said polynucleotide is set forth by SEQ ID NO:5.

64. A method of determining if a drug molecule is capable of inducing or accelerating development of fast progression of liver fibrosis in an individual, comprising comparing a metabolism rate of the drug molecule by a CYP2D6 and a poor metabolizing variant of said CYP2D6, wherein poor metabolism of the drug molecule by said poor metabolizing variant of said CYP2D6 and not said CYP2D6 is indicative of its capability of inducing or accelerating development of fast progression of liver fibrosis in the individual.

65. A method of determining if an individual is predisposed to fast progression of liver fibrosis, the method comprising determining a presence or absence, in a homozygous or heterozygous form, of at least one fast progression liver fibrosis-associated genotype in a locus selected from the group consisting of CYP3A5, CYP2E1 and APO E or in neighboring loci of the individual, said neighboring loci being in linkage disequilibrium with said locus, thereby determining if the individual is predisposed to fast progression of liver fibrosis.

66. A kit for determining if an individual is predisposed to fast progression of liver fibrosis, the kit comprising at least one reagent for determining a presence or absence in a homozygous or heterozygous form, of at least one fast progression liver fibrosis-associated genotype in a locus selected from the group consisting of CYP3A5, CYP2E1 and APO E or in neighboring loci of the individual, said neighboring loci are in linkage disequilibrium with said locus.

67. The method of claim 65, wherein said at least one fast progression liver fibrosis-associated genotype in said CYP3A5 locus is an adenosine nucleotide-containing allele at nucleotide coordinate 174 of SEQ ID NO:18.

68. The method of claim 65, wherein said at least one fast progression liver fibrosis-associated genotype in said CYP2E1 locus is a thymidine nucleotide-containing allele at nucleotide coordinate 1772 of SEQ ID NO:17.

69. The method of claim 65, wherein said at least one fast progression liver fibrosis-associated genotype in said APO E locus is a cytosine nucleotide-containing allele at nucleotide coordinate 55 of SEQ ID NO:19.


SEQUENCE LISTING (19 sequences)

SEQ ID NO: 1 - DNA, 347 bp, Homo sapiens
  misc_feature (100)..(100): G to A mutation  of CYP2D6*4
ccaaccactccggtgggtgatgggcagaagggcacaaagcgggaactgggaaggcgggggacggggaaggcgaccccttacccgcatctcccacccccangacgcccctttcgccccaacggtctcttggacaaagccgtgagcaacgtgatcgcctccctcacctgcgggcgccgcttcgagtacgacgaccctcgcttcctcaggctgctggacctagctcaggagggactgaaggaggagtcgggctttctgcgcgaggtgcggagcgagagaccgaggagtctctgcagggcgagctcccgagaggtgccggggctggactggggcctcggaagagcaggatt

SEQ ID NO: 2 - DNA, 17 bp, Artificial sequence
  Single strand DNA oligonucleotide
ccaaccactccggtggg

SEQ ID NO: 3 - DNA, 19 bp, Artificial sequence
  Single strand DNA oligonucleotide
aatcctgctcttccgaggc

SEQ ID NO: 4 - protein, 497 aa, Homo sapiens
MGLEALVPLAVIVAIFLLLVDLMHRRQRWAARYPPGPLPLPGLGNLLHVDFQNTPYCFDQLRRRFGDVFSLQLAWTPVVVLNGLAAVREALVTHGEDTADRPPVPITQILGFGPRSQGVFLARYGPAWREQRRFSVSTLRNLGLGKKSLEQWVTEEAACLCAAFANHSGRPFRPNGLLDKAVSNVIASLTCGRRFEYDDPRFLRLLDLAQEGLKEESGFLREVLNAVPVLLHIPALAGKVLRFQKAFLTQLDELLTEHRMTWDPAQPPRDLTEAFLAEMEKAKGNPESSFNDENLCIVVADLFSAGMVTTSTTLAWGLLLMILHPDVQRRVQQEIDDVIGQVRRPEMGDQAHMPYTTAVIHEVQRFGDIVPLGVTHMTSRDIEVQGFRIPKGTTLITNLSSVLKDEAVWEKPFRFHPEHFLDAQGHFVKPEAFLPFSAGRRACLGEPLARMELFLFFTSLLQHFSFSVPTGQPRPSHHGVFAFLVTPSPYELCAVPR

SEQ ID NO: 5 - DNA, 1655 bp, Homo sapiens
gctgagagtgtcctgcctggtcctctgtgcctggtggggtgggggtgccaggtgtgtccagaggagcccatttggtagtgaggcaggtatggggctagaagcactggtgcccctggccgtgatagtggccatcttcctgctcctggtggacctgatgcaccggcgccaacgctgggctgcacgctacccaccaggccccctgccactgcccgggctgggcaacctgctgcatgtggacttccagaacacaccatactgcttcgaccagttgcggcgccgcttcggggacgtgttcagcctgcagctggcctggacgccggtggtcgtgctcaatgggctggcggccgtgcgcgaggcgctggtgacccacggcgaggacaccgccgaccgcccgcctgtgcccatcacccagatcctgggtttcgggccgcgttcccaaggggtgttcctggcgcgctatgggcccgcgtggcgcgagcagaggcgcttctccgtctccaccttgcgcaacttgggcctgggcaagaagtcgctggagcagtgggtgaccgaggaggccgcctgcctttgtgccgccttcgccaaccactccggacgcccctttcgccccaacggtctcttggacaaagccgtgagcaacgtgatcgcctccctcacctgcgggcgccgcttcgagtacgacgaccctcgcttcctcaggctgctggacctagctcaggagggactgaaggaggagtcgggctttctgcgcgaggtgctgaatgctgtccccgtcctcctgcatatcccagcgctggctggcaaggtcctacgcttccaaaaggctttcctgacccagctggatgagctgctaactgagcacaggatgacctgggacccagcccagcccccccgagacctgactgaggccttcctggcagagatggagaaggccaaggggaaccctgagagcagcttcaatgatgagaacctgtgcatagtggtggctgacctgttctctgccgggatggtgaccacctcgaccacgctggcctggggcctcctgctcatgatcctacatccggatgtgcagcgccgtgtccaacaggagatcgacgacgtgatagggcaggtgcggcgaccagagatgggtgaccaggctcacatgccctacaccactgccgtgattcatgaggtgcagcgctttggggacatcgtccccctgggtgtgacccatatgacatcccgtgacatcgaagtacagggcttccgcatccctaagggaacgacactcatcaccaacctgtcatcggtgctgaaggatgaggccgtctgggagaagcccttccgcttccaccccgaacacttcctggatgcccagggccactttgtgaagccggaggccttcctgcctttctcagcaggccgccgtgcatgcctcggggagcccctggcccgcatggagctcttcctcttcttcacctccctgctgcagcacttcagcttctcggtgcccactggacagccccggcccagccaccatggtgtctttgctttcctggtgaccccatccccctatgagctttgtgctgtgccccgctagaatggggtacctagtccccagcctgctccctagccagaggctctaatgtacaataaagcaatgtggtagttcc

SEQ ID NO: 6 - DNA, 9432 bp, Homo sapiens
gaattcaagaccagcctggacaacttggaagaacccggtctctacaaaaaatacaaaattagctgggattgggtgcggtggctcatgcctataatcccagcactttgggagcctgaggtgggtggatcacctgaagtcaggagttcaagactagcctggccaacatggtgaaaccctatctctactgaaaatacaaaaagctagacgtggtggcacacacctgtaatcccagctacttaggaggctgaggcaggagaattgcttgaagcctagaggtgaaggttgtagtgagccgagattgcatcattgcacaatggaggggagccaccagcctgggcaacaagaggaaatctccgtctccaaaaaaaaaaaaaaaaaaaaaagaattaggctgggtggtgcctgtagtcccagctacttgggaggcagggggtccacttgatgtcgagactgcagtgagccatgatcctgccactgcactccggcctgggcaacagagtgagaccctgtctaaagaaaaaaaaaataaagcaacatatcctgaacaaaggatcctccataacgttcccaccagatttctaatcagaaacatggaggccagaaagcagtggaggaggacgaccctcaggcagcccgggaggatgttgtcacaggctggggcaagggccttccggctaccaactgggagctctgggaacagccctgttgcaaacaagaagccatagcccggccagagcccaggaatgtgggctgggctgggagcagcctctggacaggagtggtcccatccaggaaacctccggcatggctgggaagtggggtacttggtgccgggtctgtatgtgtgtgtgactggtgtgtgtgagagagaatgtgtgccctaagtgtcagtgtgagtctgtgtatgtgtgaatattgtctttgtgtgggtgattttctgcgtgtgtaatcgtgtccctgcaagtgtgaacaagtggacaagtgtctgggagtggacaagagatctgtgcaccatcaggtgtgtgcatagcgtctgtgcatgtcaagagtgcaaggtgaagtgaagggaccaggcccatgatgccactcatcatcaggagctctaaggccccaggtaagtgccagtgacagataagggtgctgaaggtcactctggagtgggcaggtgggggtagggaaagggcaaggccatgttctggaggaggggttgtgactacattagggtgtatgagcctagctgggaggtggatggccgggtccactgaaaccctggttatcccagaaggctttgcaggcttcaggagcttggagtggggagagggggtgacttctccgaccaggcccctccaccggcctaccctgggtaagggcctggagcaggaagcaggggcaagaacctctggagcagcccatacccgccctggcctgactctgccactggcagcacagtcaacacagcaggttcactcacagcagagggcaaaggccatcatcagctccctttataagggaagggtcacgcgctcggtgtgctgagagtgtcctgcctggtcctctgtgcctggtggggtgggggtgccaggtgtgtccagaggagcccatttggtagtgaggcaggtatggggctagaagcactggtgcccctggccgtgatagtggccatcttcctgctcctggtggacctgatgcaccggcgccaacgctgggctgcacgctacccaccaggccccctgccactgcccgggctgggcaacctgctgcatgtggacttccagaacacaccatactgcttcgaccaggtgagggaggaggtcctggagggcggcagaggtgctgaggctcccctaccagaagcaaacatggatggtgggtgaaaccacaggctggaccagaagccaggctgagaaggggaagcaggtttgggggacgtcctggagaagggcatttatacatggcatgaaggactggattttccaaaggccaaggaagagtagggcaagggcctggaggtggagctggacttggcagtgggcatgcaagcccattgggcaacatatgttatggagtacaaagtcccttctgctgacaccagaaggaaaggccttgggaatggaagatgagttagtcctgagtgccgtttaaatcacgaaatcgaggatgaagggggtgcagtgacccggttcaaaccttttgcactgtgggtcctcgggcctcactgcctcaccggcatggaccatcatctgggaatgggatgctaactggggcctctcggcaattttggtgactcttgcaaggtcatacctgggtgacgcatccaaactgagttcctccatcacagaaggtgtgacccccacccccgccccacgatcaggaggctgggtctcctccttccacctgctcactcctggtagccccgggggtcgtccaaggttcaaataggactaggacctgtagtctggggtgatcctggcttgacaagaggccctgaccctccctctgcagttgcggcgccgcttcggggacgtgttcagcctgcagctggcctggacgccggtggtcgtgctcaatgggctggcggccgtgcgcgaggcgctggtgacccacggcgaggacaccgccgaccgcccgcctgtgcccatcacccagatcctgggtttcgggccgcgttcccaaggcaagcagcggtggggacagagacagatttccgtgggacccgggtgggtgatgaccgtagtccgagctgggcagagagggcgcggggtcgtggacatgaaacaggccagcgagtggggacagcgggccaagaaaccacctgcactagggaggtgtgagcatggggacgagggcggggcttgtgacgagtgggcggggccactgccgagacctggcaggagcccaatgggtgagcgtggcgcatttcccagctggaatccggtgtcgaagtgggggcggggaccgcacctgtgctgtaagctcagtgtgggtggcgcggggcccgcggggtcttccctgagtgcaaaggcggtcagggtgggcagagacgaggtggggcaaagcctgccccagccaagggagcaaggtggatgcacaaagagtgggccctgtgaccagctggacagagccagggactgcgggagaccagggggagcatagggttggagtgggtggtggatggtggggctaatgccttcatggccacgcgcacgtgcccgtcccacccccaggggtgttcctggcgcgctatgggcccgcgtggcgcgagcagaggcgcttctccgtgtccaccttgcgcaacttgggcctgggcaagaagtcgctggagcagtgggtgaccgaggaggccgcctgcctttgtgccgccttcgccaaccactccggtgggtgatgggcagaagggcacaaagcgggaactgggaaggcgggggacggggaaggcgaccccttacccgcatctcccacccccaggacgcccctttcgccccaacggtctcttggacaaagccgtgagcaacgtgatcgcctccctcacctgcgggcgccgcttcgagtacgacgaccctcgcttcctcaggctgctggacctagctcaggagggactgaaggaggagtcgggctttctgcgcgaggtgcggagcgagagaccgaggagtctctgcagggcgagctcccgagaggtgccggggctggactggggcctcggaagagcaggatttgcatagatgggtttgggaaaggacattccaggagaccccactgtaagaagggcctggaggaggaggggacatctcagacatggtcgtgggagaggtgtgcccgggtcagggggcaccaggagaggccaaggactctgtacctcctatccacgtcagagatttcgattttaggtttctcctctgggcaaggagagagggtggaggctggcacttggggagggacttggtgaggtcagtggtaaggacaggcaggccctgggtctacctggagatggctggggcctgagacttgtccaggtgaacgcagagcacaggagggattgagaccccgttctgtctggtgtaggtgctgaatgctgtccccgtcctcctgcatatcccagcgctggctggcaaggtcctacgcttccaaaaggctttcctgacccagctggatgagctgctaactgagcacaggatgacctgggacccagcccagcccccccgagacctgactgaggccttcctggcagagatggagaaggtgagagtggctgccacggtggggggcaagggtggtgggttgagcgtcccaggaggaatgaggggaggctgggcaaaaggttggaccagtgcatcacccggcgagccgcatctgggctgacaggtgcagaattggaggtcatttgggggctaccccgttctgtcccgagtatgctctcggccctgctcaggccaaggggaaccctgagagcagcttcaatgatgagaacctgcgcatagtggtggctgacctgttctctgccgggatggtgaccacctcgaccacgctggcctggggcctcctgctcatgatcctacatccggatgtgcagcgtgagcccatctgggaaacagtgcaggggccgagggaggaagggtacaggcgggggcccatgaactttgctgggacacccggggctccaagcacaggcttgaccaggatcctgtaagcctgacctcctccaacataggaggcaagaaggagtgtcagggccggaccccctgggtgctgacccattgtggggacgcatgtctgtccaggccgtgtccaacaggagatcgacgacgtgatagggcaggtgcggcgaccagagatgggtgaccaggctcacatgccctacaccactgccgtgattcatgaggtgcagcgctttggggacatcgtccccctgggtgtgacccatatgacatcccgtgacatcgaagtacagggcttccgcatccctaaggtaggcctggcgccctcctcaccccagctcagcaccagcacctggtgatagccccagcatggctactgccaggtgggcccactctaggaaccctggccacctagtcctcaatgccaccacactgactgtccccacttgggtggggggtccagagtataggcagggctggcctgtccatccagagcccccgtctagtggggagacaaaccaggacctgccagaatgttggaggacccaacgcctgcagggagagggggcagtgtgggtgcctctgagaggtgtgactgcgccctgctgtggggtcggagagggtactgtggagcttctcgggcgcaggactagttgacagagtccagctgtgtgccaggcagtgtgtgtcccccgtgtgtttggtggcaggggtcccagcatcctagagtccagtccccactctcaccctgcatctcctgcccagggaacgacactcatcaccaacctgtcatcggtgctgaaggatgaggccgtctgggagaagcccttccgcttccaccccgaacacttcctggatgcccagggccactttgtgaagccggaggccttcctgcctttctcagcaggtgcctgtggggagcccggctccctgtccccttccgtggagtcttgcaggggtatcacccaggagccaggctcactgacgcccctcccctccccacaggccgccgtgcatgcctcggggagcccctggcccgcatggagctcttcctcttcttcacctccctgctgcagcacttcagcttctcggtgcccactggacagccccggcccagccaccatggtgtctttgctttcctggtgagcccatccccctatgagctttgtgctgtgccccgctagaatggggtacctagtccccagcctgctccctagccagaggctctaatgtacaataaagcaatgtggtagttccaactcgggtcccctgctcacgccctcgttgggatcatcctcctcagggcaaccccacccctgcctcattcctgcttaccccaccgcctggccgcatttgagacaggggtacgttgaggctgagcagatgtcagttacccttgcccataatcccatgtcccccactgacccaactctgactgcccagattggtgacaaggactacattgtcctggcatgtggggaaggggccagaatgggctgactagaggtgtcagtcagccctggatgtggtggagagggcaggactcagcctggaggcccatatttcaggcctaactcagcccaccccacatcagggacagcagtcctgccagcaccatcacaacagtcacctcccttcatatatgacaccccaaaacggaagacaaatcatggcgtcagggagctatatgccagggctacctacctcccagggctcagtcggcaggtgccagaacgttccctgggaaggccccatggaagcccaggactgagccaccaccctcagcctcgtcacctcaccacaggactggctacctctctgggccctcagggatgctgctgtacagacccctgaccagtgacgagttcgcactcagggccaggctggcgctggaggaggacacttgtttggctccaaccctaggtaccatcctcccagtagggatcaggcagggcccacaggcctgccctagggacaggagtcaaccttggacccataaggcactggggcgggcagagaaggaggaggtggcatgggcagctgagagccagagaccctgaccctagtccttgctctgccattaccccgtgtgaccccgggcccacccttccccacccttccccaccccgggcttctgtttccttctgccaacgagaaggctgcttcacctgccccgagtcctgtcttcctgctctgccttctggggctgtggcccttgctggcctggagccccaaccaagggcagggactgctgtcctccacgtctgtcctcaccgacataatgggctgggctgggcacacaggcagtgcccaagagtttctaatgagcatatgattacctgagtcctgggcagaccttcttagggaacagcctgggacagagaaccacagacactctgaggagccaccctgaggcctcttttgccagaggaccctacagcctccctggcagcagttccgccagcatttctgtaaatgccctcatgccagggtgcggcccggctgtcagcacgagagggacgttggtctgtcccctggcaccgagtcagtcagaagggtggccagggcccccttgggcccctccagagacaatccactgtggtcacacggctcggtggcaggaagtgctgttcctgcagctgtggggacagggagtgtggatgaagccaggctgggtttgtctgaagacggaggccccgaaaggtggcagcctggcctatagcagcagcaactcttggatttattggaaagattttcttcacggttctgagtcttgggggtgttagaggctcagaaccagtccagccagagctctgtcatgggcacgtagacccggtcccagggcctttgctctttgctgtcctcagaggcctctgcaaagtagaaacaggcagccttgtgagtcccctcctgggagcaaccaaccctccctctgagatgccccggggccaggtcagctgtggtgaaaggtagggatgcagccagctcagggagtggcccagagttcctgcccacccaaggaggctcccaggaaggtcaaggcacctgactcctgggctgcttccctcccctcccctccccaggtcaggaaggtgggaaagggctggggtgtctgtgaccctggcagtcactgagaagcagggtggaagcagccccctgcagcacgctgggtcagtggtcttaccagatggatacgcagcaacttccttttgaacctttttattttcctggcaggaagaagagggatccagcagtgagatcaggcaggttctgtgttgcacagacagggaaacaggctctgtccacacaaagtcggtggggccaggatgaggcccagtctgttcacacatggctgctgcctctcagctctgcacagacgtcctcgctcccctgggatggcagcttggcctgctggtcttggggttgagccagcctccagcactgcctccctgccctgctgcctcccactctgcagtgctccatggctgctcagttggacccacgctggagacgttcagtcgaagccccgggctgtccttacctcccagtctggggtacctgccacctcctgctcagcaggaatggggctaggtgcttcctcccctggggacttcacctgctctccctcctgggataagacggcagcctcctccttgggggcagcagcattcagtcctccaggtctcctgggggtcgtgacctgcaggaggaataagagggcagactgggcagaaaggccttcagagcacctcatcctcctgttctcacactggggtgtcacagtcctgggaagttcttccttttcagttgagctgtggtaaccttgtgagtttcctggagggggcctgccactacccttgggactccctgccgtgtgtctgggtctaactgagctctgaaaggagagagccccagccctgggccttccaggggaagccttacctcagaggttggcttcttcctactcttgactttgcgtctctgcagagggaggtgggaggggtgacacaaccctgacacccacactatgagtgatgagtagtcctgccccgactggcccatcctttccaggtgcagtcccccttactgtgtctgccaagggtgccagcacagccgccccactccaggggaagaggagtgccagcccttaccacctgagtgggcacagtgtagcatttattcattagcccccacactggcctgaccatctcccctgtgggctgcatgacaaggagagagaacaggctgaggtgagagctactgtcaacacctaaacctaaaaaatctataattgggctgggcagggtggctcacgcctgtaatcccagcactttgggaggccgagatgggtggatcacctgaggtcagatgttcgagaccagcctggccaacatggtgaaaccccgtctctactaaaaatacaaaaaattagctgggcgtggtggtgggtgcctgtaatcccagctactcaggaggctgaggcaggagaattgcttgaacctgggaggcagaggctgcagtgagccgagatcgcatcattgcactccagcctggtcaacaagagtgaaactgtcttaaaaaaaaaatctataattgatatctttagaaagataaaactttgcattcatgaaataagaataggagggtctaaaataaaaatgttcaaacacccaccaccactaattcttgacaaaaatatagtctgggtgccttagctcatgcctgtaatcccagcattttgggaggctaaggcaggaggattgtttgagcctaggaattc

SEQ ID NO: 7 - DNA, 22 bp, Artificial sequence
  Single strand DNA oligonucleotide
gtccaagagaccgttggggcga

SEQ ID NO: 8 - DNA, 15 bp, Artificial sequence
  Single strand DNA oligonucleotide
aggggcgtcctgggg

SEQ ID NO: 9 - DNA, 15 bp, Artificial sequence
  Single strand DNA oligonucleotide
aggggcgtcttgggg

SEQ ID NO: 10 - DNA, 18000 bp, Homo sapiens
gaggcctggcgacaattcaagtgtggtgagcgccggcaggccagcagtactgggggacccggtgccccctctgcagctgctggcccaggtgctaagcccctcactgcctggggccagaggcaccagccggccgctccgagtgcagggcccgctgagcccctgcccacccagaactggtgctggcccgcgagcaacccaggttcccgcacacgcctctccctccatacctccccgcaagcagagggagccggctccagcctccaccagtccagagaggggctcccacagtgcagcgctgggctgaagggctcctcaagtgtggtcagagcagaagctgaggccgaggaggcgctgagagcgagcgaggaccgccagcacgttgacacctctcaccctcaccacaggactggccacctctctgggccctcagggatgctgctgtccggacccctgaccagtgacgagtttgcactcagggccaggctggcgctggaggaggacacttgtttggctccaaccctaggtaccatcctcccagtagggatcaggcagggcccacaggcctgccctagggacaggagtcaaccttggacccataaggcactggggcgggtagagaaggaggaggtggcatgggcagctgagagccagagaccctgaccctagtccttgctctgccattaccccgtgtgaccccgggcccacccttccccacccttccccacccttccccaccccgggcttctgtttcccttctgccaacgagaaggctgcttcacctgccccgagtcctgtcttcctgctctgccttctggggctgtggcccttgctggcctggagccccaaccaagggcagggactgctgtcctccacgtctgtcctcaccgacataatgggctgggctgggcacacaggcagtgcccaagagtttctaatgagcatatgattacctgagtcctgggcagaccttcttagggaacagcctgggacagagaaccacagacactctgaggagccacctgaggcctcttttgccagaggaccctacagcctccctggcagcagttccgccagcatttctgtaaatgccctcatgccagggtgcggcccggctgtcagcacgagagggacgttggtctgtcccctggcaccgagtcagtcagaagggtggccagggcccccttgggcccctccagagacaatccactgtggtcacacggctcggtggcaggaagtgctgttcctgcagctgtggggacagggagtgtggatgaagccaggctgggtttgtctgaagacggaggccccgaaaggtggcagcctggcctatagcagcagcaactcttggatttattggaaagattttcttcacggttctgagtcttgggggtgttagaggctcagaaccagtccagccagagctctgtcatgggcacgtagacccggtcccagggcctttgctctttgctgtcctcagaggcctctgcaaagtagaaacaggcagccttgtgagtcccctcctgggagcaaccaaccctccctctgagatgccccggggctaggtcagctgtggtgaaaggtagggatgcagccagctcaggggagtggcccagagttcctgcccacccaaggaggctcccaggaaggtcaaggcacctgactcctgggctgcttccctcccctcccctccccaggtcaggaaggtgggaaagggctggggtgtctgtgaccctggcagtcactgagaagcagggtggaagcagccccctgcagcacgctgggtcagtggtcttaccagatggatacgcagcaacttccttttgaacctttttattttcctggcaggaagaagagggatccagcagtgagatcaggcaggttctgtgttgcacagacagggaaacaggctctgtccacacaaagtcggtggggccaggatgaggcccagtctgttcacacatggctgctgcctctcagctctgcacagacgtcctcgctcccctgggatggcagcttggcctgctggtcttggggttgagccagcctccagcactgcctccctgccctgctgcctcccactctgcagtgctccatggctgctcagttggacccacgctggagacgttcagtcgaagccccgggctgtccttacctcccagtctggggtacctgccacctcctgctcagcaggaatggggctaggtgcttcctcccctggggacttcacctgctctccctcctgggataagacagcagcctcctccttgggggcagcagcattcagtcctccaggtctcctgggggtcgtgacctgcaggaggaataagagggcagactgggcagaaaggccttcagagcacctcatcctcctgttctcacactggggtgtcacagtcctgggaagttcttccttttcagttgagctgtggtaaccttgtgagtttcctggagggggcctgccactacccttgggactccctgccgtgtgtctgggtctaactgagctctgaaaggagagagccccagccctgggccttccaggggaagccttacctcagaggttggcttcttcctactcttgactttgcgtctctgcagagggaggtgggaggggtgacacaaccctgacacccacactatgagtgatgagtagtcctgccccgactggcccatcctttccaggtgcagtcccccttactgtgtctgccaagggtgccagcacagccaccccactccaggggaagaggagtgccagcccttacccacctgagtgggcacagtgtagcatttattcattagcccccacactggcctgaccgtctcccctgtgggctgcatgacaaggagagagaacaggctgaggtgagagctactgtcaacacctaaacctaaaaaatctataattgggctgggcagggtggctcacgcctgtaatcccagcactttgggaggccgagatgggtggatcacctgaggtcagatgttcgagaccagcctggccaacatggtgaaaccccgtctctactaaaaatacaaaaaattagctgggcgtggtggtgggtgcctgtaatcccagctactcaggaggctgaggcaggagaattgcttgaacctgggaggcagaggttgcagtgagccgagatcacaccattgcactccagtctgggtgataagtatgaaacgccatctccaaaacaaaagaaaagcctaattccccaagaactgtcagtctttcacctgtctgctagctcccagggagaccccacttgccagggctgtctacatttgtcctgagatctcttctggtgggaacagcactttcctcaggaaagtttgttgaaagtcatcagatccatgattgaaaatcgaagctgcctgtggtgatggataacagctggggttaaaaagcagcagctggggcatgagcggtccacagtgagtttttgttgttgtttttgtttttttgggtgggggatggggtcttgctaggtctcaaactcctggcctcaagtcatcctcccattacagccttctgagtcactgacactacaggtgtgagccaccatgtccagcttgtagtggttttgaacagctcttgccccttcttgggaatctaggtgccctgcacgtgggtaaggctgtctgcagctgtgcccatattcaggaaggccggcaaggccctgagccctcacccgtgactgacctgaggtgctgtgcagacagcaggtgacggctaagggaaagttgagcactgcctagccgagcactgaagccacgcccggcacacagagagagacccactcggcaaagacttcgcttccaggcacctaaggaactctctgaccagtcattagctgaccactgccgtaactgaagagcggcttcagtggccacagctcgcagggaatggagacattaatgcttagtcagaattagttcagaaaagtcacccagcaaagaaacagctccaacaggcaacaacaacaacacatccttggcagggaagagaatctgacttccggagttgccacattatcgcccgtgaaatgtccaggttttaacaaattatgagacatggaaaggaaaccgaaaggacgacccagacacgggaaaagtcaccaatgggaccagcccgatgctgcaattgctagacaaagacgttcagtcagctcatttaaatatgttcaaagacctaaaacatgctgcatctgaggctgcaccggctggaacctgctgatctcggaagctaagcatggtcaggcctggctagtacttcaaagggagaaaccacgtgtaggcctggtgcagtggctcacacctataatcctagcactctgggaagctgaggcccgtggattgcttgagcccaggagtttgagagcagcttgggaaatgtggtgagacccccatctctacaaaaaatttaaaaaattagctggctgcctatggtcccagcctctcaggatgctgaggtaggaggatcacttcagcccaggaagttgaggctgcagtgagccatgactgcatcactgcactccagcttgggcgacagagagaccctctcccaagaaaaagaaaagaaccatgtcaaaagaactaacgaaagtgtgggaacaatgtctcaccaattagagaatatcaataatgggatgaaccttataaaaaggggctgggcatggtggctcatgcctataatcccagcactttgggaggctgaggcgggcatatcatgaggtcaagagattgagaccagcctggccaacatggtgaaaccccgtctctacttaaaatacaaaaattagccgggcgtggtggcacgtgcctgtaatcccagctactcgggaggctgaggcaggagaatcgcttgaacccgagaggcagagattgcagtgagccgagattgcaccactgcactacagcctgggtgacagagcgatactccaaaaaacaaaacaaaacaaaaaacaaaaaaaaagttgaaaaaggaaccaaataaaaattctggagttgtagggtaaaataaatgaaaattcatcccaggggcccaagagcagattggaacaattggaagaaagagcctgtgactatggagagaggccacctgaggtagtcccctctgaggaacaggaacaagcatgaagagcaatgcacagagatccagagacctggagacgccgtcaagctttccgacatacacgcaatgggagtcccaggaaagaagacagggagaaaggagtaaaggaatagttgaagaattaatggctgaaaaacctcccaaatctgatgaaaaatattaatctgtacatccaaaaagctcatcaaactccaagtagggtaaactcaaagagatcttcagccatacgcatcatcataatcactgtcaaaagacagatttttctttttttagaattttaaatgtaccttttaatttgctcctggggcaaagagccaggactggtactagagcagtgtctgggatgagaagaatttaataaaatgggattaggtccaatggttgggttaggggaggcaacctgctcgggaggatcagcctcaacctatccatgcagcagggcctccacctgtccctctccgtagtcccacacctggaacccagagccatctgcctcttcccagatcatggccgacagcactccaccggactgctgctggagcaggcacaggattcacttattgagggctgtggcctggcacagatcatagcctatacccagggacagttgtgtcacttctgccaccaccacatccgccttctgcagccacatcaagtaccactcatggatgagcccgtcacccccagcggacttatcaaccccgcgtccagctccacagccgccacgtgctcggtgagcactggctccaagcatggcagctgccatacaatccacctgtagagggcccggtcctcctgtcctcagtggatgatcccatagaagtccagagctcggcagctgccctcccacaaaagacaggattttgaaagcagcaagagagaagagacgtatcaggtagtcacagtggctcaggcctgtaatcccagcactttgggaggcccaggtgggaggatcgcttcaccccaggaattcaagaccagcctggacaacttggaagaaccgggtctctacaaaaaatacaaaattagctgggattgggtgcggtggctcatgcctataatcccagcactttgggagcctgaggtgggtggatcacctgaagtcaggagttcaagactagcctggccaacatggtgaaaccctatctctactgaaaatacaaaaagctagacgtggtggcacacacctgtaatcccagctacttaggaggctgaggcaggagaattgcttgaagcctagaggtgaaggttgtagtgagccgagattgcatcattgcacaatggaggggagccaccagcctgggcaacaagaggaaatctccgtctccaaaaaaaaaaaaaaaaaaaaaaaaaaaggattaggctgggtggtgcctgtagtcccagctacttgggaggcagggggtccacttgatgtcgagactgcagtgagccatgatcctgccactgcactccggcctgggcaacagagtgagaccctgtctaaagaaaaaaaaaataaagcaacatatcctgaacaaaggatcctccataacgttcccaccagatttctaatcagaaacatggaggccagaaagcagtggaggaggacgaccctcaggcagcccgggaggatgttgtcacaggctggggcaagggccttccggctaccaactgggagctctgggaacagccctgttgcaaacaagaagccatagcccggccagagcccaggaatgtgggctgggctgggagcagcctctggacaggagtggtcccatccaggaaacctccggcatggctgggaagtggggtacttggtgccgggtctgtatgtgtgtgtgactggtgtgtgtgagagagaatgtgtgctctaagtgtcagtgtgagtctgtgtatgtgtgaatattgtctttgtgtgggtgattttctgcatgtgtaatcgtgtccctgcaagtgtgaacaagtggacaagtgtctgggagtggacaagagatctgtgcaccatcaggtgtgtgcatagcgtctgtgcatgtcaagagtgcaaggtgaagtgaagggaccaggcccatgatgccactcatcatcaggagctctaaggccccaggtaagtgccagtgacagataagggtgctgaaggtcactctggagtgggcaggtgggggtagggaaagggcaaggccatgttctggaggaggggttgtgactacattagggtgtatgagcctagctgggaggtggatggccgggtccactgaaaccctggttatcccagaaggctttgcaggcttcaggagcttggagtggggagagggggtgacttctccgaccaggcccctccaccggcctaccctgggtaagggcctggagcaggaagcaggggcaagaacctctggagcagcccatacccgccctggcctgactctgccactggcagcacagtcaacacagcaggttcactcacagcagagggcaaaggccatcatcagctccctttataagggaagggtcacgcgctcggtgtgctgagagtgtcctgcctggtcctctgtgcctggtggggtgggggtgccaggtgtgtccagaggagcccatttggtagtgaggcaggtatggggctagaagcactggtgcccctggccgtgatagtggccatcttcctgctcctggtggacctgatgcaccggcgccaacgctgggctgcacgctacccaccaggccccctgccactgcccgggctgggcaacctgctgcatgtggacttccagaacacaccatactgcttcgaccaggtgagggaggaggtcctggagggcggcagaggtcctgaggatgccccaccaccagcaaacatgggtggtgggtgaaaccacaggctggaccagaagccaggctgagaaggggaagcaggtttgggggacttcctggagaagggcatttatacatggcatgaaggactggattttccaaaggccaaggaagagtagggcaagggcctggaggtggagctggacttggcagtgggcatgcaagcccattgggcaacatatgttatggagtacaaagtcccttctgctgacaccagaaggaaaggccttgggaatggaagatgagttagtcctgagtgccgtttaaatcacgaaatcgaggatgaagggggtgcagtgacccggttcaaaccttttgcactgtgggtcctcgggcctcactgctcaccggcatggaccatcatctgggaatgggatgctaactggggcctctcggcaattttggtgactcttgcaaggtcatacctgggtgacgcatccaaactgagttcctccatcacagaaggtgtgacccccacccccgccccaggatcaggaggctgggtctcctccttccacctgctcactcctggtagccccgggggtcgtccaaggttcaaataggactaggacctgtagtctggggggatcctggcttgacaagaggccctgaccctccctctgcagttgcggcgccgcttcggggacgtgttcagcctgcagctggcctggacgccggtggtcgtgctcaatgggctggcggccgtgcgcgaggcgctggtgacccacggcgaggacaccgccgaccgcccgcctgtgcccatcacccagatcctgggtttcgggccgcgttcccaaggcaagcagcggtggggacagagacagatttccgtgggacccgggtgggtgatgaccgtagtccgagctgggcagagagggcgcggggtcgtggacatgaaacaggccagcgagtggggacagcgggccaagaaaccacctgcactagggaggtgtgagcatggggacgagggcggggcttgtgacgagtgggcggggccactgccgagacctggcaggagcccaatgggtgaggctggcgcatttcccagctggaatccggtgtcgaagtggggggcggggaccgcacctgtgctgtaagctcagtgtgggtggcgcggggcccgcggggtcttccctgagtgcaaaggcggtcagggtgggcagagacgaggtggggcaaagccctgccccagccaagggagcaaggtggatgcacaaagagtgggccctgtgaccagctggacagagccagggactgcgggagaccagggggagcatagggttggagtgggtggtggatggtggggctaatgccttcatggccacgcgcacgtgcccgtcccacccccaggggtgttcctggcgcgctatgggcccgcgtggcgcgagcagaggcgcttctccgtctccaccttgcgcaacttgggcctgggcaagaagtcgctggagcagtgggtgaccgaggaggccgcctgcctttgtgccgccttcgccaaccactccggtgggtgatgggcagaagggcacaaagcgggaactgggaaggcgggggacggggaaggcgaccccttacccgcatctcccacccccaggacgcccctttcgccccaacggtctcttggacaaagccgtgagcaacgtgatcgcctccctcacctgcgggcgccgcttcgagtacgacgaccctcgcttcctcaggctgctggacctagctcaggagggactgaaggaggagtcgggctttctgcgcgaggtgcggagcgagagaccgaggagtctctgcagggcgagctcccgagaggtgccggggctggactggggcctcggaagagcaggatttgcatagatgggtttgggaaaggacattccaggagaccccactgtaagaagggcctggaggaggaggggacatctcagacatggtcgtgggagaggtgtgcccgggtcagggggcaccaggagaggccaaggactctgtacctcctatccacgtcagagatttcgattttaggtttctcctctgggcaaggagagagggtggaggctggcacttggggagggacttggtgaggtcagtggtaaggacaggcaggccctgggtctacctggagatggctggggcctgagacttgtccaggtgaacgcagagcacaggagggattgagaccccgttctgtctggtgtaggtgctgaatgctgtccccgtcctcctgcatatcccagcgctggctggcaaggtcctacgcttccaaaaggctttcctgacccagctggatgagctgctaactgagcacaggatgacctgggacccagcccagcccccccgagacctgactgaggccttcctggcagagatggagaaggtgagagtggctgccacggtggggggcaagggtggtgggttgagcgtcccaggaggaatgaggggaggctgggcaaaaggttggaccagtgcatcacccggcgagccgcatctgggctgacaggtgcagaattggaggtcatttgggggctaccccgttctgtcccgagtatgctctcggccctgctcaggccaaggggaaccctgagagcagcttcaatgatgagaacctgtgcatagtggtggctgacctgttctctgccgggatggtgaccacctcgaccacgctggcctggggcctcctgctcatgatcctacatccggatgtgcagcgtgagcccatctgggaaacagtgcaggggccgagggaggaagggtacaggcgggggcccatgaactttgctgggacacccggggctccaagcacaggcttgaccaggatcctgtaagcctgacctcctccaacataggaggcaagaaggagtgtcagggccggaccccctgggtgctgacccattgtggggacgcatgtctgtccaggccgtgtccaacaggagatcgacgacgtgatagggcaggtgcggcgaccagagatgggtgaccaggctcacatgccctacaccactgccgtgattcatgaggtgcagcgctttggggacatcgtccccctgggtgtgacccatatgacatcccgtgacatcgaagtacagggcttccgcatccctaaggtaggcctggcgccctcctcaccccagctcagcaccagcccctggtgatagccccagcatggctactgccaggtgggcccactctaggaaccctggccacctagtcctcaatgccaccacactgactgtccccacttgggtggggggtccagagtataggcagggctggcctgtccatccagagcccccgtctagtggggagacaaaccaggacctgccagaatgttggaggacccaacacctgcagggagagggggcagtgtgggtgcctctgagaggtgtgactgcgccctgctgtggggtcggagagggtactgtggagcttctcgggcgcaggactagttgacagagtccagctgtgtgccaggcagtgtgtgtcccccgtgtgtttggtggcaggggtcccagcatcctagagtccagtccccactctcaccctgcatcttctgcccagggaacgacactcatcaccaacctgtcatcggtgctgaaggatgaggccgtctgggagaagcccttccgcttccaccccgaacacttcctggatgcccagggccactttgtgaagccggaggccttcctgcctttctcagcaggtgcctgtggggagcccggctccctgtccccttccgtggagtcttgcaggggtatcacccaggagccaggctcactgacgcccctcccctccccacaggccgccgtgcatgcctcggggagcccctggcccgcatggagctcttcctcttcttcacctccctgctgcagcacttcagcttctcggtgcccactggacagccccggcccagccaccatggtgtctttgctttcctggtgaccccatccccctatgagctttgtgctgtgccccgctagaatggggtacctagtccccagcctgctccctagccagaggctctaatgtacaataaagcaatgtggtagttccaactcgggtcccctgctcacgccctcgttgggatcatcctcctcagggcaaccccacccctgcctcattcctgcttaccccaccgcctggccgcatttgagacaggggtacgttgaggctgagcagatgtcagttacccttgcccataatcccatgtcccccactgacccaactctgactgcccagattgatgacaaggactacattgtcctggcatgtggggaaggggccagaatgggctgactagaggtgtcagtcagccctggatgtggtggagagggcaggactcagcctggaggcccatatttcaggcctaactcagcccaccccacatcagggacagcagtcctgccagcaccatcacagtcacctcccttcatatatgacaccccaaaacggaagacaaatcatggcgtcagggagctataggccagggctacctacctcccagggctcagtcggcaggtgccagaacgttccctgggaaggccccatggaagcccaggactgagccaccaccctcagcctcgtcacctcaccacaggactggctacctctctgggccctcagggacgctgctgtacagacccctgaccagtgacgagttcgcactcagggccaggctggcgctggaggaggacacttgtttggctccaaccctaggtaccatcctcccagtagggatcaggcagggcccacaggcctgccctagggacaggagtcaaccttggacccataaggcactggggcgggcagagaaggaggaggtggcatgggcagctgagagccagagaccctgaccctagtccttgctctgccattaccccgtgtgaccccgggcccacccttccccacccttccccacccttccccaccccgggcttctgtttcccttctgccaacgagaaggctgcttcacctgccccgagtcctgtcttcctgctctgccttctggggctgtggcccttgctggcctggagccccaaccaagggcagggactgctgtcctccacgtctgtcctcaccgacataatgggctgggctgggcacacaggcagtgcccaagagtttctaatgagcatatgattacctgagtcctgggcagaccttcttagggaacagcctgggacagagaaccacagacactctgaggagccacctgaggcctcttttgccagaggaccctacagcctccctggcagcagttccgccagcatttctgtaaatgccctcatgccagggtgcggcccggctgtcagcacgagagggacgttggtctgtcccctggcaccgagtcagtcagaagggtggccagggcccccttgggcccctccagagacaatccactgtggtcacacggctcggtggcaggaagtgctgttcctgcagctgtggggacagggagtgtggatgaagccaggctgggtttgtctgaagacggaggccccgaaaggtggcagcctggcctatagcagcagcaactcttggatttattggaaagattttcttcacggttctgagtcttgggggtgttagaggctcagaaccagtccagccagagctctgtcatgggcacgtagacccggtcccagggcctttgctctttgctgtcctcagaggcctctgcaaagtagaaacaggcagccttgtgagtcccctcctgggagcaaccaaccctccctctgagatgccccggggctaggtcagctgtggtgaaaggtagggatgcagccagctcaggggagtggcccagagttcctgcccacccaaggaggctcccaggaaggtcaaggcacctgactcctgggctgcttccctcccctcccctccccaggtcaggaaggtgggaaagggctggggtgtctgtgaccctggcagtcactgagaagcagggtggaagcagccccctgcagcacgctgggtcagtggtcttaccagatggatacgcagcaacttccttttgaacctttttattttcctggcaggaagaagagggatccagcagtgagatcaggcaggttctgtgttgcacagacagggaaacaggctctgtccacacaaagtcggtggggccaggatgaggcccagtctgttcacacatggctgctgcctctcagctctgcacagacgtcctcgctcccctgggatggcagcttggcctgctggtcttggggttgagccagcctccagcactgcctccctgccctgctgcctcccactctgcagtgctccatggctgctcagttggacccacgctggagacgttcagtcgaagccccgggctgtccttacctcccagtctggggtacctgccacctcctgctcagcaggaatggggctaggtgcttcctcccctggggacttcacctgctctccctcctgggataagacagcagcctcctccttgggggcagcagcattcagtcctccaggtctcctgggggtcgtgacctgcaggaggaataagagggcagactgggcagaaaggccttcagagcacctcatcctcctgttctcacactggggtgtcacagtcctgggaagttcttccttttcagttgagctgtggtaaccttgtgagtttcctggagggggcctgccactacccttgggactccctgccgtgtgtctgggtctaactgagctctgaaaggagagagccccagccctgggccttccaggggaagccttacctcagaggttggcttcttcctactcttgactttgcgtctctgcagagggaggtgggaggggtgacacaaccctgacacccacactatgagtgatgagtagtcctgccccgactggcccatcctttccaggtgcagtcccccttactgtgtctgccaagggtgccagcacagccaccccactccaggggaagaggagtgccagcccttacccacctgagtgggcacagtgtagcatttattcattagcccccacactggcctgaccgtctcccctgtgggctgcatgacaaggagagagaacaggctgaggtgagagctactgtcaacacctaaacctaaaaaaatctataattgggctgggcagggtggctcacgcctgtaatcccagcactttgggaggccgagatgggtggatcacctgaggtcagatgttcgagaccagcctggccaacatggtgaaaccccgtctctactaaaaatacaaaaaattagccaggtgtggtggcgggtgcctgtaatcccagctactcaggaggctgaggcaggagaatcgattgaacccaggaagtggaggctgcaatgagccaagatcgcgtcattgcactccagcctggtcaacaagagtgaaactgtcttaaaaaaaaaatctataattgatatctttagaaagataaaactttgcattcatgaaataagaataggagggtctaaaataaaaatgttcaaacacccaccaccactaattcttgacaaaaatatagtctgggtgccttagctcatgcctgtaatcccagcattttgggaggctaaggcaggaggattgtttgagcctaggaattcaacaccagcctgggccacctaaggagaccccatctctacaaaaaattaaaatactggctgggtgtggtggcacacacctgtagttccagctgcttgggaggctgaggtgggaggatcacttgagtccaggaacaaagctgcagtgaactgtgatcgtgccactgcactccagcctgggcaacagagaaagaccttgccttaaaaataaaaaatataataataggaatgcaaaatctaatcaaagtatagaagctaaacttgaaaaaaatattttccagaaagaacagagaagaggtcaggagctccaacagctaaattgttgtttagatgtttctgaaacaggcagcagagacaacagactaggaggcaaggaaagatgtctaataaatacgtttcttttttgtcaagacaagttctcacagaggaagaacatgagtttccagtagagaaggaaacaccaagtgttcatgacaatgaatgaaggggacccagccccaattttgttgtcaagaaatttcacaacactgaggacagagtggaacctaaaaacttccagagagaaaaaagtctgagcttcaggaattcaacattcatcagacttctcaacaccaacctttgaagctataagataatgaagaccttcaaaatctgagagaaaatatttccaatctagaattctatacctagccaaatgctatgcaagtatgaattgaggtcttttcggatacataaatgtctcaagactacccctcaggaagcaaccggaggttgtacttcactaaaataaaggagaaatagaaaagaagataacatgggacccagcacaacaggcagggagagcccctgagcataagggtgaatggggagctcaggaggacagctgggcagcagacctccagggtgccccatccagatggaatcagggagatggagggctcctgaggtatgtctccatgaaaatgatcatatggagaaatgacctgatctgtctaaatgtactgcaaagagatttctatttttggcagaaaatttggatgaattaattatataatagatgcacaaaaaactaaagaaagagaagaagaaaaactaaaatcatgactcaactgggactactgtctacattttttgttttgagaaagagtcttgctctgttgcccagactggagtgcagtgatcacgtttcattgcagcctccacaacctgtgctcaagtaagtgactctcttacctcagcctccttagtagctgggatcacagggcaccaccacactcagctaacttttttttttaaatagacagtgtctcccaatgttgtccaggctggtctcgaactcctggactcaagcgatcctcccatgttgacctctcaagtagttgggattacagtcatgagccactgtgcctgacctagctaatttttttctgatttatttatttattttttgtacagagtctcactatgttgaccaggctggtctggaacttctgagctcaagtgatcctcctgccttcgcctcccaaagtgcagagattataggtgtaaactatcacgcctggcctgtttacatagtttaataatgtaaatcttcaataccgatctaataaaaattgaaatatgccttttagaatggctttcaaagataacaaatgctggagaggatgtagaacaactggaacctctcggttattgctggtgagacagccgctttgaaaaagtttgagtttcttacaaaattaaacttacacttacacttaccatatgacccaaaaattccactgcttgctctttactcaagtataaggaaaatctatgtacacacaaaacttgtacgtgaatatttattaatagtcattttatgccccaaactagaaatagtccaaatgttctggaacatccatacaacggaccaccactcaataaaaggaacaaactacggatacacgtgactagatgaatctcaaatgctttgtgctaagtaaaataaaccagactgaaaaggctaccatacgtttccatttatatgacaatcttgcaaagtcaaaaccacaggaacaggaaactgttcactgattgccagggtgtgggagtaggaggaagggctgactacaggtgactatggaggatttttttttttctgagacggagtctctgtcgcccaggctggagtgtactggcacgatctcggctcactgcaacatccacctcctgggtttaaggtatttttagtagagacggggtttcactatgttggccaggctggtctcaaactcctgacctcaggtgatccacccgcttcggcctcccaaagtgctgggattataggcgtgagccaccgaggccagccgcttttttttttttaaagacagagtcttgctgtgtcactcaggctggagtgcagtggcgtgatcccagctcactgcagccttaacatcctgcactcaagtgatc

SEQ ID NO: 11 - DNA, 21 bp, Artificial sequence
  Single strand DNA oligonucleotide
ccagtcgagtctacattgtca

SEQ ID NO: 12 - DNA, 21 bp, Artificial sequence
  Single strand DNA oligonucleotide
ccagttagaagacagaatgaa

SEQ ID NO: 13 - DNA, 24 bp, Artificial sequence
  Single strand DNA oligonucleotide
ctttaaagagctcttttgtctctc

SEQ ID NO: 14 - DNA, 20 bp, Artificial sequence
  Single strand DNA oligonucleotide
ccaggaagccagactttgat

SEQ ID NO: 15 - DNA, 23 bp, Artificial sequence
  Single strand DNA oligonucleotide
tccaaggagctgcaggcggcgca

SEQ ID NO: 16 - DNA, 31 bp, Artificial sequence
  Single strand DNA oligonucleotide
acagaattcgccccggcctggtacactgcca

SEQ ID NO: 17 - DNA, 14776 bp, Homo sapiens
  misc_feature (1532)..(1532): polymorphic nucleotide n can be G or C
cccccattgaaaaattgtctttctgatctttataaacaattatttaatatccagtaaaatcttctctatattgctttactagtgagttctattaaaattttgaagcacagaaaattcccctacagtataaagtatccccagtcacagagaagacaggggttttgcaatgatttctagaatagtgcaatttttatgcaagaacctaatataacacaaaaattatagcccgattttatttgtgggtatagatgcaaaattactaaaaatactattaacaagttgaatccttagggtgttaaaagagtatcactccatgaacgagttggttgtgatgtggaactatgaggtacttttatgatacaatataaaaatttatggtaattttatggtacattgtgagacagtgttttcttctagcatcatactagcaggtctatggagaaaaatcacaggattgtctcaatcaaaaaaagatttcattaacccaactctcatccctgataaacactgttagttatctagagaaagaagaaaattgtcccaatacagtcacctctttgccacacccagccaacagcagacgtgatggaagcctgaagaacaccctgccacgggcacaggcagaggcacaggcaccctgtcgtcctgattatttcaccttgtcacgggcagaggcacaggcaccctgtcgtcctgattatttcaccttgtcacaggcacaggcaccctgtcgtcctgattatttcaccttgtcacaggcacaggcactctgtcgtcctgattatttcaccttgtcacgggcagaggcacaggcactctgtcatcctgattatttcaccttgtcctagagtgtcctgccaatgggacagatgcaaaacaaataaaagccccggcttctgaaaagaagcacacagaaatgtcattattttcaaacgaggtgttcccgtatataaaatttgatgttggttgggcatctaacagtattatggccagaggactcagaccacagctgcatccctgtgaggcacagactctccagggcacgcgggtcccgctgggatgtgcacactcaggtgagctgcacagacaaggtgtcctcagcccaggggagccagaggcctgctctgcctctccaccctgatgcttcctgttctcaccccaccaaagccaaggcttcaatttcagtctgtggggagctgactctgctgctctcaagcactagaagaaggaaccagtaatcgaggaaacttgtggaccccaatggtgtctgtcccggccaggcctggctgggcccacacaggacaacagggttcaggggtctggacagctgtttctgcccagggaattgtccctgccacctcacactggccactggaaaggaaagagaggaggaggcggcaggctaacccacccgtgagccagtcgagtctacattgtcagttctcacctcgaggggtgccaaaaaccagagggaagcaaaggcccctgaagcctctgccagaggccaacgccccttcttggttcaggagagntgcagtgttaggtgcagcacaaccaatgacttgcttatgtggctaataaattgtcaagagaaaaactgggttagaatgcaatatatagtatgtagtctcatttttgtataaatacaagtatagaatggcataactcaaaatccacaagtgatttggctggattgtaaatgacttttattttcttcatttctcatcatattttctattatacataaagattcattgttaatataaaagtanaaaattgcaacctatgaattaagaacttctatatattgccagttagaagacagaatgaaaaacattctcttcattctaaccacacacacaaaaaactccacaaaatacctatggactaccttcatagaaggtggaagagggtctgtatgaagaaaatgcttaatacatgaaagaagaagctagtcaatgtggaggtctattgtgcgccgggatcaacaaagacaagatatgtttaaaatggtgttctaaatttaccctaatgtaaaacaaatccaataaaactctaatgtgattttttaagaatttaaatttggaataattccaaagaacaatttttcttaatttctacagccagaatatatacctttaaaaaaaatgaaaacagagattaactttctcagaattggttgactcactctttccttttatttttcttccatggaattttccagttaacttgagaaagtggaatcgaattccgatgttgaattttccttctggccccattcatgtggcaggtggtgattcaggtactactgggggctgctcagacaaacctcctcatcagacatcaagaggctgttgcaccaggagggccggtaccgtgtctagaggtggtcggcatggggttggagttgtattacataaaccctactccaaacaaatgcatggggatgtggctggagttccccgttgtctaaccagtgccaaagggcaggtcggtacctcaccccacgttcttaactatgggttggcaacatgttcctggatgtgtttgctggcacagtgacaggtgctagcaaccagggtgttgacacagtccaactccatcctcaccaggtcactggctggaacccctgggggccaccattgcgggaatcagcctttgaaacgatggccaacagcagctaataataaaccagtaatttgggatagacgagtagcaagagggcattggttggtgggtcaccctccttctcagaacacattataaaaaccttcctttccacaggattgtcctcccgggctggcagcagggccccagcggcaccatgtctgccctcggagtcaccgtggccctgctggtgtgggcggccttcctcctgctggtgtccatgtggaggcaggtgcacagcagctggaatctgcccccaggccctttcccgcttcccatcatcgggaacctcttccagttggaattgaagaatattcccaagtccttcacccgggtaagagaaatagtgttgattttagggagaataactcagcaattggatctggtatgtgtgtattcaactcatttgcagacaaattgtggttgttcaataccagcctgttgtgaattacctgaattgatagcatcctggagcgacactcaaaatgtgtcgcctgtggtgcagctggagcccggagcctgcgtgccaggccccggaggcccccgccgtgccttgtcctggggctgatgatggggaggccggcgaggccgggctgctgcgacgccaggataaccgggctggcggccagatgcgcactcgctgggcgtccgcctgtgtttgccaaagcacgagttgaaacgtgaagtgttgggccagcccgtgtggcaccaatacctgccgcctacgactgttgtgaacactgaatgggccaacaaacctaaacgttaaatgaactgataacgccgtcagcacggagcaggcgctgggtgtttgcgctcttgcgcgtgcgctgctgtggggcgcaggctgacggcgggcgggggtcgcctgctccagctcgggctcccgcgccagaaccgggtccagaaccttgattccggaagcgggcaacggggtggttggtgggcgcgcctgagggaagggacgtgaggagccggagtccgcggagttgccgcggagttgtccgcggagtccaggcgggtggggagcagagcagctggaaccccccgagcgccctgcagacgcagcagcctcttgaggggagggtctcccccacctcgggctggacaaagacagcttttccccacgtccctctgggttctctagagcaacagcaatacccgcccggcaggtgtggcttagagccccgcacctcctcgccgcgcgcgggcctgacttctagccacgggtctccgcagttggcccagcgcttcgggccggtgttcacgctgtacgtgggctcgcagcgcatggtggtgatgcacggctacaaggcggtgaaggaagcgctgctggactacaaggacgagttctcgggcagaggcgacctccccgcgttccatgcgcacagggacaggggtgagtccgcgtccctggcacggagcggggggtgcataacacgccccgggacagttacgggcgctagccacgtcggcgatggccaaataataaactaacagtaatattatagtaatagcatccgaaggatgagatcaggattaggcgatggcccccgcgcgttgcctgccgagcgaggcgcactgagtcgcccaggaatccggcctctcggcgactgtgcgggagagttttatggggatgggcggggctgcttctgagcaggagtcgccgcccccacccccaccgttccgcctctgggccgcaggctcctcccgggagcgctttcccctcctgttcaaccgccggggtacaggtggcttcgtccaccgaggtcccctcacccacgctgaggcgtcggaagctgcggacactgctcgcttcagggctttgctcagctgcagctggtgacctccagagagggagtctctgatgtcccgctggggtggatgtcctgagaccgggaagggggaagagacccactgaaatcctatctcccagcctcacctctgctgtctcctccacgcttcctgtctccagagccccgagttcagcataagcagaaagcggcctgttccctctctagggagaggagggttgcggtctggaggtctggctcgtctttatctgcgcattctcccagcctcctggcttcagacctcagcgaggcggcggctgcggccggctctcctcttcctgcctgcagacctggcctgctgcttctttctccttcctccctccctgcctgccctgcggtttcaaagtagattagaaataacagtgtcccacatggaagcctctacttcttcctgggtcaactttgatgacgaggctccagaaaacctttgcaatgctgtgtggaatttttaaatcggtgagctcgtgctcttgccctatttatttgtccagcgtacatttctgaacattgtgaacgtcgaatgggccaacaaatctaaaaattaaatgagctgataaagaacgccgtcagcacagagcagacgctgggtgttcgcgctcttgagcgtgcgctctgcggggcgcgggctggtggcgggcgggggtcgccggctccagctcaggttcccgcgccaggaccgcgtccagaaccttgtctccggaagcgggcaacggggtggttgtatcacaattagtggcatttggttttccttcttctgcattgtgggttttacttctctggggttgccaaaaacaaaattaaccatctcagtccttgtcgttaacgcaggagaagcattactggaggaggctctggggttctgtggttgaggagctcagttctggttccggggagcccttatctgccacccacgggtccaaggcacagtcggaggcagcagggaggggagcggaattcacatcaacacagatggggctcaaggggactttgctgcctctgcctggagggtctaaagtttcattttcatatgacccgcagggcgcagactggcggaaaattagcagagccctgggcatgggctgcacctggccttaagggacaatgatggaaatattccttattagcacaatactgagcacaggctgtgtgataatgtgtcaagggaactgcagacatcctttcagaaaaagttcataaaacggagaaagtttggttcccaacctagatttttaacctgttgaactctgtctaaatgggtcatctcgggatgtcctccactcaacatgaccacagtctgcccctctgtcccacctgtctcctcagtccttcctccccacctttcaggatgaaatgaaaccctcagtccagctgcacccctgccccacccacctcatctcatgtgccctcccgcccctctcaggccggacagccttgcttctggaacacacgagcacagcttcaccaggcactttctgagcaccctgcaggcgcctcccaggagtggtcagtggtcaatcagctaatgaagctgcataggacatgacccttgtttaccgcagaatgcccagagctggcaggatgtcttatatgcaggaagtacccaaaatgtatttattgaggaagtgatgatggataagaggaagacggagagcgagggagagaggggctaggggccctgcggtgtaaagggggtgtggctgggagtgtgcaggggaacagggatcatttcaaggttcctatctgggagaaaataaaaaggtttacagttagttgagataagcgtgggaatatgcgaacatttttaaagaataaaaagtttagctttaaatttgttgattccaaatgtgttcatactctcgggaggatccatcaagcaactcttgggaggagagacagggcagggcaggccttgacagctcagaagggcgcagtagggacagttcttggttttcccagctctgatgctttgcacagtcgcttgtgtgacctgcaagattttagtgaagaaacttgctgtggagtcggaaagctgcaagttgaggtgtgtgtggtgtgagggttaaaaatctgtgagaacagaatgaatggcttttcaagaatgttgtcgatagataggaaagaggtgggaggtgttcttggagtggccatatgtggttttatgtagcatggggaagactcagcagaaaggaaaaagaaagaaggtaaattgacagcatgaagtagagcacccaggagaggctacatgtgatgaagaaaccacagtgcagactgtgaggaccccagaaaggctcctccccaaaacctgaccagtggccggtgctggcagctcccaggctgggacaccctctgtctctctgtccctctgccccctctgtcacttctttatacacctgtaaatcctgccctgctctccaaggccctctgtagcccatttctccccaaaatgggtatttagaataaccttctgctggcccctctgccttaggaatcatttttaataatggacctacctggaaggacatccggcggttttccctgaccaccctccggaactatgggatggggaaacagggcaatgagagccggatccagagggaggcccacttcctgctggaagcactcaggaagacccaaggtgcgtatctgctgcctagcagggcccagtcctcttgcagaccagcggtgtggggagccctggctgggactcctagactgcatctgaaccacagggacctacggacaaggagagggtctcgtgagtccccagatactgcattttacaactctaggttccagctacacagttcagggagcaagggtggccattaaacacgtgacttgtatcctaaatactgttgaaaagcaaaggaaactcaaacaggttcagacattcactatctttcgtaaactggcagttttcagggcaccttctcacaggccttggtgaacctcagtgggtgactgagcaggtggaggagtctcctcacccccatcttctggttgccctgactgcctgttttgtaggccagcctttcgaccccaccttcctcatcggctgcgcgccctgcaacgtcatagccgacatcctcttccgcaagcattttgactacaatgatgagaagtttctaaggctgatgtatttgtttaatgagaacttccacctactcagcactccctggctccaggtgaagccactttcctctttcatcagtcatcaactgtagagtttacgttagaaaaagaaggaaaatttgggttatatgtgatagacaggactgcaaaagccaaacaacatagcttcgaggggtgtttgattagacagcccaaatattcctcccagagacatctctggggccccacgcaccccctttcctaacgtcaggatgtgtatcgacctgtgtgtgcacatttgccatgcagagtttgcactgctgaggagaatggtgcccaagaaggacactgttgacccaaaatattccaaataaacaatgattacagccacaaattcaggtttggagaaagttgttggtccaacacacacaattatgttgcatccagaaaaaagtagtaaaatatttttttccctctctagctttacaataattttcccagctttctacactacttgcctggaagccacagaaaagtcataaaaaatgtggctgaagtaaaagagtatgtgtctgaaagggtgaaggagcaccatcaatctctggaccccaactgtccccgggacctcaccgactgcctgctcgtggaaatggagaaggtaggctcggcctcccatgatgtgggctctccggggtgggcagagaatgcacaatttcagatttacagagtgagctgcacttgctggtgtccagacctcccaccgcagcatgctctgagtttcatacacacactcttggcttcagcatgaccactggacgcaagtcagcctgcctggctgccaagctggcctggggtttggggcacatgggcgggacgcttagctctctccaggccctgctgctcaaccctttctagtctgcagactttgagaattgcattttgtctgaggagaagccctcagccttccttgtgggcatgcactccccaactgtgcgcacgtgcaggacttccaggcctccccagcttcatccacctgcaggtgctcaggatcctgatcccctgcccccttcccaccttggtgaaacttcttgtatccttgtcttgtcctttcctatggcttgtggctcaagaacaaatgtggagcccacactgatttcccaggactgtctgagcatcttctccaccagtttggcccctcgtggcagcagacactagccctgtagcaggaggggttagcaggagccgtttagctcctgcctgagctatgaccaaggtcagggggatctcacctctcccaggatggccctcatgctgtggagggagacagagccctggcctgccctcagcagatttctgggagcctcagtttccctggctgtgagtggagatgactctgtctgtcacagctccaagtcacagttccactgggagagcctcttggacactgtctcctgtgtccctgtggagctgggaggtggctggttctgtgctgaaaggagacaagcagccccttctctccggtctgtctccggtatcacaggaaaagcacagtgcagagcgcttgtacacaatggacggtatcaccgtgactgtggccgacctgttctttgcggggacagagaccaccagcacaactctgagatatgggctcctgattctcatgaaataccctgagatcgaaggtaggcaagtgactgaagggacaccgtgcgtgcggctgcatctccctggatggccagccttgcacattttaggctgcagctttctgtctgaagctgcttgttaaccctcatggtgatgtggtgagatggctggatgcactgctgtgaggggaggtgttatggtctgtgctgaacactggtactcttgcacactggttggtccataccccactaagacacccctggttgcagaaaagaacatcccaacaccagagtggagagaggtggcagggtctgcattctgctccataaataacctctttatgacagagaagataatgtcccagttccccccaagtaagacctggtcttctaggcagagcaggtggggaggttggagctggaggggagggtccttgctggggcgtcttcctcaaatgcggacgtgaggagggaagtccaggaagaagcagctacagctccccctggacccttgtcgttccttccacagggctcctcccagcggcacctggggcagctgggactctgtgcctggaggaggtgtgaaaggtctgggtctaggtgggcagagggtcatgccctgagaaacacccatctgggccaagtagaggtgatgtgagggcaccgcatgcaaacaggccagtcagggttgggtccaagtaaaggggaggaaagggagctgcagcctggctggagagtgccggggggcccagagcccctgcctctcgctgggctggaaacagggctgggcagcctctgcccgaggcagttcacagcctgagtggtgtgtgccgccctcctcctgaagctgctgctaatggtcacttgtggtcttaaggctcgtcagttcctgaaagcaggtattataggctatgaagttatttcccccaagaaagtcgacatgtgatggatccagggtcagaccctggcttttcttgttctttccttcttcttcttctttttatttatttattttttttttgaggggacagggtctcactctgttgcccaggctggagtgcggtgatgcaatcatggctcattgtagcttctacctattgggctcaagcgatcctcccacctcagcctcccaagtaactgggccacaggtgcacaccaccacacccagctgattaaaaatttaaaaaaattattttggctgggcacagtggctcatacctgtaatcctggcactttgggaggctgaggcaggcgatcacgaggtcaggagttcgagaccttcctggccaacatgatgaaaccctgtctctcctaaaatacaaaaaagtagccgggtgtggtggcacgcgcctatagtcacagctactcaggaggctgaggcaggagaatcgcttcaacctcagaggcacagggtgcagtgatccgagattgcaccccactgcactctagcctgacaacagagcaagaatcagtctaaaaaaaaaattgtagagacaagttgttactatgttttgtaggctggtcttgaactcctgggctcaagtcatcctcctgccttggcctcccaaagtgctggggttacaggtgtggccaccgtgccccatccctggcctttgctttttcaatcacatggaaatgtgaagggtgaaggagccaaaagtttagggaaggaatcattgtatggatctgcagtgattataagagaactttcgactactctgcactaggggaaccatggaatcaaaaaatgttttaaattattatttatgaggaggttccaatatagacaaaaggaaaataaatatgattgacatgtatatatccattgccaaattgaacgtttattaacattttgcgatacttccatcagagctcttaaaaagaaaatgtgttacagagccagccaaagtctacctcctcacatctccccacctctctcaccagaaatggcttcagaattgctgtgtggctttgcacttttaacagttgttaattatcagcacagtattcatattattgctgtatgtgtttaatattttacctgggtactgtacataacattttgcagcttggttttttcactcaacatatgatgatgttccatgggaactccaaacacggggaggctaggcgacttgctcaaggcagctgttacctctgtcagaaagacagaggctttcagattcaagaagtagaccctgcatgtctgattctgttctgtaaacccccttcatactcagaagcatgcaataaacaagcctggggtaattatcaatgcaaaggttaccctcccagaagaaatttccaaaacactttcattattctctgctcttgacatgaagagaactgaataagccatcatcaactgagataatggatgccaaaacatccagtaaataacctcatagagcttagctctcactaagtttttggagcattttccagtaattcaaaggacctggggaaccttaagcactgcttaggatgctccataaacatcttctgcgtgggtaggggagtggatggatggctggatgggtgggtggatggacggacggatggatggatggatggatggatggatggttggatggatgggtgggtggatggatggatgggtcaatggatgtgtggatggatggaagggtgggtggatgggtggatggctggctggttgggtgggtgggtggatggatgcatgggtggatggatggaggatggatggatggatggaggggtgtatagatggaggggtggatggatgtgtaggtgggcagatggataaaagcgtgattgaatagatgggtggatgatgggtggatgcccaactggccaggaaccaatccctgaaatttgtcccattcatatcttggcagagaagctccatgaagaaattgacagggtgattgggccaagccgaatccctgccatcaaggataggcaagagatgccctacatggatgctgtggtgcatgagattcagcggttcatcaccctcgtgccctccaacctgccccatgaagcaacccgagacaccattttcagaggatacctcatccccaaggttaagcaatgagcctgcagcacacagcatgaacaccatcctatcactaatcgccttcctgccagggagcaggatgggggccccaagacccttccctttggcaggggtcactgaggggaagggctggccccactcccaccctgtgggatactgcatctccaggagtgctcacattggcctggtgaccagagaggtggaggaaatctggaaaagagcctcagcagatagtgcctgggactgtagtgaattctaatgccaggaacaaactatcacaaccagccctggggttaatcctgtgagaagattagggctttcatcttcatttagacctgacccctgactgctttctatctaatccttcactaagcaactccttcaactcgaaatatactatcctatatagcataatattcaaaacaacattcttcactgggggtttccagatgaaagcccacattttgttaacatgactcactgagacagtctttgtttctcctagggcacagtcgtagtgccaactctggactctgttttgtatgacaaccaagaatttcctgatccagaaaagtttaagccagaacacttcctgaatgaaaatggaaagttcaagtacagtgactatttcaagccattttccacaggtgagaaagatcagaggcagtaccttcccttgaggagcagcccacactcctcatctcccctccacatgtgctctgccctcgtcccaggcacccactgacaccccaaacctcactgtgtgccctgtttctattgacaacatgacccaaatgtgctcttccctgttcagagaagttacataacatcttttagcagcaatcctgggaatgaagtgttgtaggtggattttttttttcccaaagactagacattttacatcattcattgctaaattttgtttctattttaacaagacttagtgaaaagctctcaaagccatattacccaattctccctaattttaaaccagagctactaaacaaaacctaacctttggttacctagaatcatcacaggaagcatcaaagccttcctgggatgtgactcagtgattttctttgaggcacttgtcctccttcccagggcctcatcttagggattgttgtgggaagatcatacaaccaactccatacttttcacacccagtgctggagccccagcttctaacagggcactatttccctcctgtaggcatcactgatgagcactgggggtgccttctttactgggcagacatggtcttcccaacttaacaccggtttttgcagttgagctctggataattgagattgtatgaaggctggtccccgaattagtcagtgtcgctggtatccttccactcaagtacattttgtgcttcttttaataggcagagaggggtgagtcctgccctgtgatggccgtttgcccacagcctcctcctccccgcttcccctagtctcactgttaacagtgtcgtgtctctgaaactccctcagtgtctcatcaataccattgttacttctaggaaaacgagtgtgtgctggagaaggcctggctcgcatggagttgtttcttttgttgtgtgccattttgcagcattttaatttgaagcctctcgttgacccaaaggatatcgacctcagccctatacatattgggtttggctgtatcccaccacgttacaaactctgtgtcattccccgctcatgagtgtgtggaggacaccctgaaccccccgctttcaaacaagatttcgaattgtttgaggtcaggatttctcaaactgattcctttctttgcatatgagtatttgaaaataaatattttcccagaatataaataaatcatcacatgattattttaactatatgttaagtcatggaatatcttaattgtttaagtgattctcacagagaggtttttttttttttttttttttttttgagagttttgctcttgttgaccaggatggagtgcagtggcatgatcttggctcactgcaacctctgtgtcctgggttcaagtgattctcctccctcagcctcccgaatagctgggattacaggcacccaccaccatgccagctaattctttgtatttttagcagagacagggtttcaccatgttggtcaggctggtcttgaacccctgacctcaggtgatccacctacctcggcctcccaaagtgctgggattacagcatgagccaccgcgcccagccagagagaggttttaaatatatatgtttactttaatattaagttataacataattttcatgttattgaaaagctcttccatctaggatcacaccacttcagtgtcagaatcatattgaggtggggaatttgtattagtcaggtttctctaaagggacagaaacaataggatagatgtatatacgaaagggagtttattaggagaattgactcacatga

SEQ ID NO: 18 - DNA, 1440 bp, Homo sapiens
  misc_feature (174)..(174): polymorphic nucleotide n can be A or G
cctgccttcaatttttcactgacctaatattctttttgataatgaagtattttaaacatataaaacattatggagagtggcataggagatacccacgtatgtaccacccagcttaacgaatgctctactgtcatttctaaccataatctctttaaagagctcttttgtctttcngtatctcttccctgtttggaccacattacccttcatcatatgaagccttgggtggctcctgtgtgagactcttgctgtgtgtcacaccctaatgaactagaacctaaggttgctgtgtgtcgtacaactaggggtatggattacataacataatgatcaaagtctggcttcctgggtgtggctccagctgcagaatcgggctagtgaagtttaatcagctccgttgtccccacacagaacgtatgaaggtcaactccctgtgctggccatcacagatcccgacgtgatcagaacagtgctagtgaaagaatgttattctgtcttcacaaatcgaagggtaagcatccattttttgaaatttaaataatgattgatccactgattaaatttttattttgaaaaaaacatatattcacagaaggttacctaaaaaatgtacaggaaggttccatgtactcttcatcctgtcccgcccagtggtaacatcttgcaatcttgtatattgcaatatatatctagtatattcatattatcaggttggcacaaaagttaaaatggcaaactacaggctgggcataatggctcatgcctgtaatcccagcactttgggaggccgaggcaggtggatcacgaggtcaggagttcgagatcagcctgaccaacatggtgaaaccccatctctactaaaaatacaaaaattagctgcgtgtggtggcatgcgcctgtagtcccagctactcagtagtctgagacaggagaatcgcttgaacctgggaggcggaggttgcagtgagccgagatcacgccattatactccagtctgggcaacccaatgagactccatctcaaacaacaacaacaacaacaacaacaaaaaccggcaaactgcaataacttttgcaccaacctaatactatagtacaggaaattgactttgatatagtttacagagcttttcagatttcaccagttttacatgcccttgtttgtgtgtgtttatgtgtgtgggtagttctaagcaatttttcacattcgtagatttgtgcaacgaccagcaccatcaagatgcagacccattccgtcaccatgtggctccctcctgctgtcctacagtcacaacatggagtttgtctttttctctgacaggttctatatcagagcaaacttttatttatttgaggaggccaatgtattaatatttccttttatggattgttcttttggtgttaagtctgaaaatcctttgcttagccctccttcctacat

SEQ ID NO: 19 - DNA, 227 bp, Artificial sequence
  PCR product for SNP evaluation
tccaaggagctgcaggcggcgcaggcccggctgggcgcggacatggaggacgtgngcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagngcctggcagtgtaccaggccggggcgaattctgt